SEQUENCE LISTING (40 sequences)

SEQ ID NO: 1 - DNA, 3465 bp, Homo sapiens
atgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataa

SEQ ID NO: 2 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 3 - DNA, 3399 bp, Homo sapiens
atgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatga

SEQ ID NO: 4 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 5 - DNA, 3375 bp, Homo sapiens
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 6 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 7 - DNA, 3564 bp, Homo sapiens
atgcctctgcgccactgggggatggccaggggcagtaagcccgttggggatggagcccagcccatggctgccatgggaggcctgaaggtgcttctgcactgggctggtccaggcggcggggagccctgggtcactttcagtgagtcatcgctgacagctgaggaagtctgcatccacattgcacataaagttggtatcactcctccttgcttcaatctctttgccctcttcgatgctcaggcccaagtctggttgcccccaaaccacatcctagagatccccagagatgcaagcctgatgctatatttccgcataaggttttatttccggaactggcatggcatgaatcctcgggaaccggctgtgtaccgttgtgggcccccaggaaccgaggcatcctcagatcagacagcacaggggatgcaactcctggacccagcctcatttgagtacctctttgagcagggcaagcatgagtttgtgaatgacgtggcatcactgtgggagctgtcgaccgaggaggagatccaccactttaagaatgagagcctgggcatggcctttctgcacctctgtcacctcgctctccgccatggcatccccctggaggaggtggccaagaagaccagcttcaaggactgcatcccgcgctccttccgccggcatatccggcagcacagcgccctgacccggctgcgccttcggaacgtcttccgcaggttcctgcgggacttccagccgggccgactctcccagcagatggtcatggtcaaatacctagccacactcgagcggctggcaccccgcttcggcacagagcgtgtgcccgtgtgccacctgaggctgctggcccaggccgagggggagccctgctacatccgggacagtggggtggcccctacagaccctggccctgagtctgctgctgggcccccaacccacgaggtgctggtgacaggcactggtggcatccagtggtggccagtagaggaggaggtgaacaaggaggagggttctagtggcagcagtggcaggaacccccaagccagcctgtttgggaagaaggccaaggctcacaaggcagtcggccagccggcagacaggccgcgggagccactgtgggcctacttctgtgacttccgggacatcacccacgtggtgctgaaagagcactgtgtcagcatccaccggcaggacaacaagtgcctggagctgagcttgccttcccgggctgcggcgctgtccttcgtgtcgctggtggacggctatttccgcctgacggccgactccagccactacctgtgccacgaggtggctcccccacggctggtgatgagcatccgggatgggatccacggacccctgctggagccatttgtgcaggccaagctgcggcccgaggacggcctgtacctcattcactggagcaccagccacccctaccgcctgatcctcacagtggcccagcgtagccaggcaccagacggcatgcagagcttgcggctccgaaagttccccattgagcagcaggacggggccttcgtgctggagggctggggccggtccttccccagcgttcgggaacttggggctgccttgcagggctgcttgctgagggccggggatgactgcttctctctgcgtcgctgttgcctgccccaaccaggagaaacctccaatctcatcatcatgcggggggctcgggccagccccaggacactcaacctcagccagctcagcttccaccgggttgaccagaaggagatcacccagctgtcccacttgggccagggcacaaggaccaacgtgtatgagggccgcctgcgagtggagggcagcggggaccctgaggagggcaagatggatgacgaggaccccctcgtgcctggcagggaccgtgggcaggagctacgagtggtgctcaaagtgctggaccctagtcaccatgacatcgccctggccttctacgagacagccagcctcatgagccaggtctcccacacgcacctggccttcgtgcatggcgtctgtgtgcgcggccctgaaaatatcatggtgacagagtacgtggagcacggacccctggatgtgtggctgcggagggagcggggccatgtgcccatggcttggaagatggtggtggcccagcagctggccagcgccctcagctacctggagaacaagaacctggttcatggtaatgtgtgtggccggaacatcctgctggcccggctggggttggcagagggcaccagccccttcatcaagctgagtgatcctggcgtgggcctgggcgccctctccagggaggagcgggtggagaggatcccctggctggcccccgaatgcctaccaggtggggccaacagcctaagcaccgccatggacaagtgggggtttggcgccaccctcctggagatctgctttgacggagaggcccctctgcagagccgcagtccctccgagaaggagcatttctaccagaggcagcaccggctgcccgagccctcctgcccacagctggccacactcaccagccagtgtctgacctatgagccaacccagaggccatcattccgcaccatcctgcgtgacctcacccggctgcagccccacaatcttgctgacgtcttgactgtgaacccggactcaccggcgtcggaccctacggttttccacaagcgctatttgaaaaagatccgagatctgggcgagggtcacttcggcaaggtcagcttgtactgctacgatccgaccaacgacggcactggcgagatggtggcggtgaaagccctcaaggcagactgcggcccccagcaccgctcgggctggaagcaggagattgacattctgcgcacgctctaccacgagcacatcatcaagtacaagggctgctgcgaggaccaaggcgagaagtcgctgcagctggtcatggagtacgtgcccctgggcagcctccgagactacctgccccggcacagcatcgggctggcccagctgctgctcttcgcccagcagatctgcgagggcatggcctatctgcacgcgcagcactacatccaccgagacctagccgcgcgcaacgtgctgctggacaacgacaggctggtcaagatcggggactttggcctagccaaggccgtgcccgaaggccacgagtactaccgcgtgcgcgaggatggggacagccccgtgttctggtatgccccagagtgcctgaaggagtataagttctactatgcgtcagatgtctggtccttcggggtgaccctgtatgagctgctgacgcactgtgactccagccagagcccccccacgaaattccttgagctcataggcattgctcagggtcagatgacagttctgagactcactgagttgctggaacgaggggagaggctgccacggcccgacaaatgtccctgtgaggtctatcatctcatgaagaactgctgggagacagaggcgtcctttcgcccaaccttcgagaacctcatacccattctgaagacagtccatgagaagtaccaaggccaggccccttcagtgttcagcgtgtgctga

SEQ ID NO: 8 - protein, 1187 aa, Homo sapiens
MPLRHWGMARGSKPVGDGAQPMAAMGGLKVLLHWAGPGGGEPWVTFSESSLTAEEVCIHIAHKVGITPPCFNLFALFDAQAQVWLPPNHILEIPRDASLMLYFRIRFYFRNWHGMNPREPAVYRCGPPGTEASSDQTAQGMQLLDPASFEYLFEQGKHEFVNDVASLWELSTEEEIHHFKNESLGMAFLHLCHLALRHGIPLEEVAKKTSFKDCIPRSFRRHIRQHSALTRLRLRNVFRRFLRDFQPGRLSQQMVMVKYLATLERLAPRFGTERVPVCHLRLLAQAEGEPCYIRDSGVAPTDPGPESAAGPPTHEVLVTGTGGIQWWPVEEEVNKEEGSSGSSGRNPQASLFGKKAKAHKAVGQPADRPREPLWAYFCDFRDITHVVLKEHCVSIHRQDNKCLELSLPSRAAALSFVSLVDGYFRLTADSSHYLCHEVAPPRLVMSIRDGIHGPLLEPFVQAKLRPEDGLYLIHWSTSHPYRLILTVAQRSQAPDGMQSLRLRKFPIEQQDGAFVLEGWGRSFPSVRELGAALQGCLLRAGDDCFSLRRCCLPQPGETSNLIIMRGARASPRTLNLSQLSFHRVDQKEITQLSHLGQGTRTNVYEGRLRVEGSGDPEEGKMDDEDPLVPGRDRGQELRVVLKVLDPSHHDIALAFYETASLMSQVSHTHLAFVHGVCVRGPENIMVTEYVEHGPLDVWLRRERGHVPMAWKMVVAQQLASALSYLENKNLVHGNVCGRNILLARLGLAEGTSPFIKLSDPGVGLGALSREERVERIPWLAPECLPGGANSLSTAMDKWGFGATLLEICFDGEAPLQSRSPSEKEHFYQRQHRLPEPSCPQLATLTSQCLTYEPTQRPSFRTILRDLTRLQPHNLADVLTVNPDSPASDPTVFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHAQHYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKYQGQAPSVFSVC

SEQ ID NO: 9 - DNA, 1956 bp, Homo sapiens
atggcggacagtgcggaactaaagcaaatggttatgagccttagagtttctgaactccaagtactgttgggctacgccgggagaaacaagcacggacgcaaacacgaacttctcacaaaagccctgcatttgctaaaggctggctgtagtcctgctgtgcaaatgaaaattaaggaactctataggcggcggttcccacagaaaatcatgacgcctgcagacttgtccatccccaacgtacattcaagtcctatgccagcaactttgtctccatctaccattccacaactcacttacgatggtcaccctgcatcatcgccattactccctgtttctcttctgggacctaaacatgaactggaactcccacatcttacatcagctcttcacccagtccatccggatataaaacttcaaaaattaccattttatgatttactggatgaactgataaaacccaccagtctagcatcagacaacagtcagcgctttcgagaaacctgttttgcatttgccttgacaccacaacaagtgcagcaaatcagtagttccatggatatttctgggaccaaatgtgacttcacagtacaggtccagttaaggttttgtttatcagaaaccagttgtccacaagaagatcacttcccacccaatctttgtgtgaaagtgaatacaaaaccttgcagccttccaggttaccttccacctacaaaaaatggcgtggaaccaaagcgacccagccgaccaattaatatcacctcacttgtccgactgtccacaacagtaccaaacacgattgttgtttcttggactgcagaaattggaagaaactattccatggcagtatatcttgtaaaacagttgtcctcaacagttcttcttcagaggttacgagcaaagggaataaggaatccggatcattctagagctttaattaaagagaagttgactgcggatccagacagtgaaatagctacaaccagcctaagggtttctctactatgtccacttggtaaaatgcggctgacaattccgtgtcgggcccttacatgttctcatctacaatgttttgacgcaactctttacattcagatgaatgagaaaaaaccaacctgggtttgtcctgtctgtgataagaaggctccatatgaacaccttattattgatggcttgtttatggaaatcctaaagtactgtacagactgtgatgaaatacaatttaaggaggatggcacttgggcaccgatgagatcaaaaaaggaagtacaggaagtttctgcctcttacaatggagtcgatggatgcttgagctccacattggagcatcaggtagcgtctcaccaccagtcctcaaataaaaacaagaaagtagaagtgattgacctaaccatagacagttcatctgatgaagaggaagaagagccatctgccaagaggacctgtccttccctatctcccacatcaccactaaataataaaggcattttaagtcttccacatcaagcatctccagtatcccgcaccccaagccttcctgctgtagacacaagctacattaatacctccctcatccaagactataggcatcctttccacatgacacccatgccttacgacttacaaggattagatttctttcctttcttatcaggagacaatcagcattacaacacctccttgcttgccgctgcagcagcagcagtttcagatgatcaagacctcctacactcgtctcggtttttcccgtatacctcctcacagatgtttcttgatcagttaagtgcaggaggcagtacttctctgccaaccaccaatggaagcagtagtggcagtaacagcagcctggtttcttccaacagcctaagggaaagccatagccacaccgtcacaaacaggagcagcacggacacggcatccatctttggcatcataccagacattatttcattggactga

SEQ ID NO: 10 - protein, 651 aa, Homo sapiens
MADSAELKQMVMSLRVSELQVLLGYAGRNKHGRKHELLTKALHLLKAGCSPAVQMKIKELYRRRFPQKIMTPADLSIPNVHSSPMPATLSPSTIPQLTYDGHPASSPLLPVSLLGPKHELELPHLTSALHPVHPDIKLQKLPFYDLLDELIKPTSLASDNSQRFRETCFAFALTPQQVQQISSSMDISGTKCDFTVQVQLRFCLSETSCPQEDHFPPNLCVKVNTKPCSLPGYLPPTKNGVEPKRPSRPINITSLVRLSTTVPNTIVVSWTAEIGRNYSMAVYLVKQLSSTVLLQRLRAKGIRNPDHSRALIKEKLTADPDSEIATTSLRVSLLCPLGKMRLTIPCRALTCSHLQCFDATLYIQMNEKKPTWVCPVCDKKAPYEHLIIDGLFMEILKYCTDCDEIQFKEDGTWAPMRSKKEVQEVSASYNGVDGCLSSTLEHQVASHHQSSNKNKKVEVIDLTIDSSSDEEEEEPSAKRTCPSLSPTSPLNNKGILSLPHQASPVSRTPSLPAVDTSYINTSLIQDYRHPFHMTPMPYDLQGLDFFPFLSGDNQHYNTSLLAAAAAAVSDDQDLLHSSRFFPYTSSQMFLDQLSAGGSTSLPTTNGSSSGSNSSLVSSNSLRESHSHTVTNRSSTDTASIFGIIPDIISLD

SEQ ID NO: 11 - DNA, 1719 bp, Homo sapiens
atggcggatttcgaagagttgaggaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggagaacaaagaagaaatgatattaataatgaactgaagcttggaacatcttctgatactgtgcaacagtga

SEQ ID NO: 12 - protein, 572 aa, Homo sapiens
MADFEELRNMVSSFRVSELQVLLGFAGRNKSGRKHDLLMRALHLLKSGCSPAVQIKIRELYRRRYPRTLEGLSDLSTIKSSVFSLDGGSSPVEPDLAVAGIHSLPSTSVTPHSPSSPVGSVLLQDTKPTFEMQQPSPPIPPVHPDVQLKNLPFYDVLDVLIKPTSLVQSSIQRFQEKFFIFALTPQQVREICISRDFLPGGRRDYTVQVQLRLCLAETSCPQEDNYPNSLCIKVNGKLFPLPGYAPPPKNGIEQKRPGRPLNITSLVRLSSAVPNQISISWASEIGKNYSMSVYLVRQLTSAMLLQRLKMKGIRNPDHSRALIKEKLTADPDSEIATTSLRVSLMCPLGKMRLTIPCRAVTCTHLQCFDAALYLQMNEKKPTWICPVCDKKAAYESLILDGLFMEILNDCSDVDEIKFQEDGSWCPMRPKKEAMKVSSQPCTKIESSSVLSKPCSVTVASEASKKKVDVIDLTIESSSDEEEDPPAKRKCIFMSETQSSPTKGVLMYQPSSVRVPSVTSVDPAAIPPSLTDYSVPFHHTPISSMSSDLPGEQRRNDINNELKLGTSSDTVQQ

SEQ ID NO: 13 - DNA, 1866 bp, Homo sapiens
atggcggatttcgaagagttgaggaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggtttggattttctttcccttattccagttgatccccagtactgtcctcctatgtttttggatagtctcacctcacccttaacagcaagcagtacgtctgtcaccaccaccagctcccatgaaagcagtactcatgttagttcatccagcagcaggagtgagacaggggtcataaccagcagtggaagtaacattcctgacatcatctcattggactaa

SEQ ID NO: 14 - protein, 621 aa, Homo sapiens
MADFEELRNMVSSFRVSELQVLLGFAGRNKSGRKHDLLMRALHLLKSGCSPAVQIKIRELYRRRYPRTLEGLSDLSTIKSSVFSLDGGSSPVEPDLAVAGIHSLPSTSVTPHSPSSPVGSVLLQDTKPTFEMQQPSPPIPPVHPDVQLKNLPFYDVLDVLIKPTSLVQSSIQRFQEKFFIFALTPQQVREICISRDFLPGGRRDYTVQVQLRLCLAETSCPQEDNYPNSLCIKVNGKLFPLPGYAPPPKNGIEQKRPGRPLNITSLVRLSSAVPNQISISWASEIGKNYSMSVYLVRQLTSAMLLQRLKMKGIRNPDHSRALIKEKLTADPDSEIATTSLRVSLMCPLGKMRLTIPCRAVTCTHLQCFDAALYLQMNEKKPTWICPVCDKKAAYESLILDGLFMEILNDCSDVDEIKFQEDGSWCPMRPKKEAMKVSSQPCTKIESSSVLSKPCSVTVASEASKKKVDVIDLTIESSSDEEEDPPAKRKCIFMSETQSSPTKGVLMYQPSSVRVPSVTSVDPAAIPPSLTDYSVPFHHTPISSMSSDLPGLDFLSLIPVDPQYCPPMFLDSLTSPLTASSTSVTTTSSHESSTHVSSSSSRSETGVITSSGSNIPDIISLD

SEQ ID NO: 15 - DNA, 1887 bp, Homo sapiens
atggcggagctgggcgaattaaagcacatggtgatgagtttccgggtgtctgagctccaggtgcttcttggctttgctggccggaacaagagtggacggaagcacgagctcctggccaaggctctgcacctcctgaagtccagctgtgcccctagtgtccagatgaagatcaaagagctttaccgacgacgctttccccggaagaccctggggccctctgatctctcccttctctctttgccccctggcacctctcctgtaggctcccctggtcctctagctcccattcccccaacgctgttggcccctggcaccctgctgggccccaagcgtgaggtggacatgcacccccctctgccccagcctgtgcaccctgatgtcaccatgaaaccattgcccttctatgaagtctatggggagctcatccggcccaccacccttgcatccacttctagccagcggtttgaggaagcgcactttacctttgccctcacaccccagcaagtgcagcagattcttacatccagagaggttctgccaggagccaaatgtgattataccatacaggtgcagctaaggttctgtctctgtgagaccagctgcccccaggaagattattttccccccaacctctttgtcaaggtcaatgggaaactgtgccccctgccgggttaccttcccccaaccaagaatggggccgagcccaagaggcccagccgccccatcaacatcacacccctggctcgactctcagccactgttcccaacaccattgtggtcaattggtcatctgagttcggacggaattactccttgtctgtgtacctggtgaggcagttgactgcaggaacccttctacaaaaactcagagcaaagggtatccggaacccagaccactcgcgggcactgatcaaggagaaattgactgctgaccctgacagtgaggtggccactacaagtctccgggtgtcactcatgtgcccgctagggaagatgcgcctgactgtcccttgtcgtgccctcacctgcgcccacctgcagagcttcgatgctgccctttatctacagatgaatgagaagaagcctacatggacatgtcctgtgtgtgacaagaaggctccctatgaatctcttatcattgatggtttatttatggagattcttagttcctgttcagattgtgatgagatccaattcatggaagatggatcctggtgcccaatgaaacccaagaaggaggcatctgaggtttgccccccgccagggtatgggctggatggcctccagtacagcccagtccaggggggagatccatcagagaataagaagaaggtcgaagttattgacttgacaatagaaagctcatcagatgaggaggatctgccccctaccaagaagcactgttctgtcacctcagctgccatcccggccctacctggaagcaaaggagtcctgacatctggccaccagccatcctcggtgctaaggagccctgctatgggcacgttgggtggggatttcctgtccagtctcccactacatgagtacccacctgccttcccactgggagccgacatccaaggtttagatttattttcatttcttcagacagagagtcagcactatggcccctctgtcatcacctcactagatgaacaggatgcccttggccacttcttccagtaccgagggaccccttctcactttctgggcccactggcccccacgctggggagctcccactgcagcgccactccggcgccccctcctggccgtgtcagcagcattgtggcccctgggggggccttgagggaggggcatggaggacccctgccctcaggtccctctttgactggctgtcggtcagacatcatttccctggactga

SEQ ID NO: 16 - protein, 628 aa, Homo sapiens
MAELGELKHMVMSFRVSELQVLLGFAGRNKSGRKHELLAKALHLLKSSCAPSVQMKIKELYRRRFPRKTLGPSDLSLLSLPPGTSPVGSPGPLAPIPPTLLAPGTLLGPKREVDMHPPLPQPVHPDVTMKPLPFYEVYGELIRPTTLASTSSQRFEEAHFTFALTPQQVQQILTSREVLPGAKCDYTIQVQLRFCLCETSCPQEDYFPPNLFVKVNGKLCPLPGYLPPTKNGAEPKRPSRPINITPLARLSATVPNTIVVNWSSEFGRNYSLSVYLVRQLTAGTLLQKLRAKGIRNPDHSRALIKEKLTADPDSEVATTSLRVSLMCPLGKMRLTVPCRALTCAHLQSFDAALYLQMNEKKPTWTCPVCDKKAPYESLIIDGLFMEILSSCSDCDEIQFMEDGSWCPMKPKKEASEVCPPPGYGLDGLQYSPVQGGDPSENKKKVEVIDLTIESSSDEEDLPPTKKHCSVTSAAIPALPGSKGVLTSGHQPSSVLRSPAMGTLGGDFLSSLPLHEYPPAFPLGADIQGLDLFSFLQTESQHYGPSVITSLDEQDALGHFFQYRGTPSHFLGPLAPTLGSSHCSATPAPPPGRVSSIVAPGGALREGHGGPLPSGPSLTGCRSDIISLD

SEQ ID NO: 17 - DNA, 1533 bp, Homo sapiens
atggcggcggagctggtggaggccaaaaacatggtgatgagttttcgagtctccgaccttcagatgctcctgggtttcgtgggccggagtaagagtggactgaagcacgagctcgtcaccagggccctccagctggtgcagtttgactgtagccctgagctgttcaagaagatcaaggagctgtacgagacccgctacgccaagaagaactcggagcctgccccacagccgcaccggcccctggaccccctgaccatgcactccacctacgaccgggccggcgctgtgcccaggactccgctggcaggccccaatattgactaccccgtgctctacggaaagtacttaaacggactgggacggttgcccgccaagaccctcaagccagaagtccgcctggtgaagctgccgttctttaatatgctggatgagctgctgaagcccaccgaattagtcccacagaacaacgagaagcttcaggagagcccgtgcatcttcgcattgacgccaagacaggtggagttgatccggaactccagggaactgcagcccggagttaaagccgtgcaggtcgtcctgagaatctgttactcagacaccagctgccctcaggaggaccagtacccgcccaacatcgctgtgaaggtcaaccacagctactgctccgtcccgggctactacccctccaataagcccggggtggagcccaagaggccgtgccgccccatcaacctcactcacctcatgtacctgtcctcggccaccaaccgcatcactgtcacctgggggaactacggcaagagctactcggtggccctgtacctggtgcggcagctgacctcatcggagctgctgcagaggctgaagaccattggggtaaagcacccggagctgtgcaaggcactggtcaaggagaagctgcgccttgatcctgacagcgagatcgccaccaccggtgtgcgggtgtccctcatctgtccgctggtgaagatgcggctctccgtgccctgccgggcagagacctgcgcccacctgcagtgcttcgacgccgtcttctacctgcagatgaacgagaagaagcccacctggatgtgccccgtgtgcgacaagccagccccctacgaccagctcatcatcgacgggctcctctcgaagatcctgagcgagtgtgaggacgccgacgagatcgagtacctggtggacggctcgtggtgcccgatccgcgccgaaaaggagcgcagctgcagcccgcagggcgccatcctcgtgctgggcccctcggacgccaatgggctcctgcccgcccccagcgtcaacgggagcggtgccctgggcagcacgggtggcggcggcccggtgggcagcatggagaatgggaagccgggcgccgatgtggtggacctcacgctggacagctcatcgtcctcggaggatgaggaggaggaggaagaggaggaggaagacgaggacgaagaggggccccggcccaagcgccgctgccccttccagaagggcctggtgccggcctgctga

SEQ ID NO: 18 - protein, 510 aa, Homo sapiens
MAAELVEAKNMVMSFRVSDLQMLLGFVGRSKSGLKHELVTRALQLVQFDCSPELFKKIKELYETRYAKKNSEPAPQPHRPLDPLTMHSTYDRAGAVPRTPLAGPNIDYPVLYGKYLNGLGRLPAKTLKPEVRLVKLPFFNMLDELLKPTELVPQNNEKLQESPCIFALTPRQVELIRNSRELQPGVKAVQVVLRICYSDTSCPQEDQYPPNIAVKVNHSYCSVPGYYPSNKPGVEPKRPCRPINLTHLMYLSSATNRITVTWGNYGKSYSVALYLVRQLTSSELLQRLKTIGVKHPELCKALVKEKLRLDPDSEIATTGVRVSLICPLVKMRLSVPCRAETCAHLQCFDAVFYLQMNEKKPTWMCPVCDKPAPYDQLIIDGLLSKILSECEDADEIEYLVDGSWCPIRAEKERSCSPQGAILVLGPSDANGLLPAPSVNGSGALGSTGGGGPVGSMENGKPGADVVDLTLDSSSSSEDEEEEEEEEEDEDEEGPRPKRRCPFQKGLVPAC

SEQ ID NO: 19 - DNA, 636 bp, Homo sapiens
atggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttga

SEQ ID NO: 20 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 21 - DNA, 678 bp, Homo sapiens
atggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaa

SEQ ID NO: 22 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 23 - DNA, 1788 bp, Homo sapiens
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 24 - protein, 595 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 25 - DNA, 1794 bp, Homo sapiens
atgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 26 - protein, 597 aa, Homo sapiens
MLSRGWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 27 - DNA, 1875 bp, Homo sapiens
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaagagcttggagtctagtgcagggaccgtggctgcgtcacctgtgagacggggtggccagaggggactgccagtgccgggtccccctgtgctgtctcctgacctgcaccaactgcctgtacttgcccccctgcacccggctgcagacacaaggaggatgtgtatgagaacctgcacactaagaacaagagggaggagaaagtga

SEQ ID NO: 28 - protein, 624 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKSLESSAGTVAASPVRRGGQRGLPVPGPPVLSPDLHQLPVLAPLHPAADTRRMCMRTCTLRTRGRRK

SEQ ID NO: 29 - DNA, 1782 bp, Homo sapiens
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatga

SEQ ID NO: 30 - protein, 593 aa, Homo sapiens [sequence omitted]

SEQ ID NO: 31 - DNA, 1383 bp, Homo sapiens
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcaggtga

SEQ ID NO: 32 - protein, 460 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR

SEQ ID NO: 33 - DNA, 45 bp, Artificial Sequence
  /note=“Description of Artificial Sequence Partial Homo sapiens wild type Jak3 cDNA sequence encoding V722”
tgggaagtgtttagtggcgtcaccatgcccatcagtgccctggat

SEQ ID NO: 34 - protein, 15 aa, Artificial Sequence
  /note=“Description of Artificial Sequence Partial Homo sapiens wild type Jak3 amino acid sequence encompassing V722”
WEVFSGVTMPISALD

SEQ ID NO: 35 - DNA, 45 bp, Artificial Sequence
  /note=“Description of Artificial Sequence Partial Homo sapiens mutant Jak3 cDNA sequence encoding I722”
tgggaagtgtttagtggcatcaccatgcccatcagtgccctggat

SEQ ID NO: 36 - protein, 15 aa, Artificial Sequence
  /note=“Description of Artificial Sequence Partial Homo sapiens mutant Jak3 amino acid sequence encompassing I722”
WEVFSGITMPISALD

SEQ ID NO: 37 - DNA, 42 bp, Artificial Sequence
  /note=“Description of Artificial Sequence Partial Homo sapiens wild type Jak3 cDNA sequence encoding S61”
tgcgtgcaggctgccaaggccagcggtgagtgcatccctagt

SEQ ID NO: 38 - protein, 14 aa, Artificial Sequence
  /note=“Description of Artificial Sequence Partial Homo sapiens wild type Jak3 amino acid sequence encompassing S61”
CVQAAKASGECIPS

SEQ ID NO: 39 - DNA, 42 bp, Artificial Sequence
  /note=“Description of Artificial Sequence Partial Homo sapiens mutant Jak3 cDNA sequence encoding C61”
tgcgtgcaggctgccaaggcctgcggtgagtgcatccctagt

SEQ ID NO: 40 - protein, 14 aa, Artificial Sequence
  /note=“Description of Artificial Sequence Partial Homo sapiens mutant Jak3 amino acid sequence encompassing C61”
CVQAAKACGECIPS

(19) United States
(12) Patent Application Publication
(10) Pub. No.: US 2017/0115291 A1
(43) Pub. Date: Apr. 27, 2017

(54) Activating JAK Kinase Biomarkers Predictive of Anti-Immune Checkpoint Inhibitor Response

(71) Applicant: Dana-Farber Cancer Institute, Inc., Boston, MA (US)

(72) Inventors: Kwok-Kin Wong, Arlington, MA (US); David Barbie, Andover, MA (US); Eliezer Van Allen, Brookline, MA (US)

(21) Appl. No.: 15/314,253

(22) PCT Filed: May 28, 2015
(86) PCT No.: PCT/US15/32823

§ 371 Date: Nov. 28, 2016

Related U.S. Application Data
(60) Provisional application No. 62/003,698, filed on May 28, 2014.

(51) Int. Cl. G01N 33/574; C07K 16/28; A61K 45/06; A61K 39/395; G01N 33/50; C12Q 1/68

(52) U.S. Cl. CPC G01N 33/574; G01N 33/5011; C12Q 1/6886; A61K 45/06; A61K 39/39558; C07K 16/2827; C12Q 2600/106; C12Q 2600/158; C12Q 2600/156; C12Q 2600/136; G01N 2500/10; A61K 2039/505

(57) ABSTRACT

The present invention is based on the identification of novel biomarkers predictive of responsiveness to anti-immune checkpoint inhibitor therapies.

### CROSS-REFERENCE TO RELATED APPLICATIONS

This application claims the benefit of U.S. Provisional Application No. 62/003,698, filed on 28 May 2014; the entire contents of said application are incorporated herein in their entirety by this reference.

### STATEMENT OF RIGHTS

This invention was made with government support under Grant Numbers R01 CA122794, R01 CA166480, R01 CA163896, R01 CA140594, U01 CA141576, and K08 CA138918-01A1 awarded by the National Institutes of Health. The U.S. government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

Immune checkpoint blockade targeting the PD-L1/PD-1 receptor interaction has been a major advance in the therapy of melanoma and other solid malignancies, such as non-small cell lung cancer (NSCLC). Although inhibiting such immune checkpoint inhibitors has been demonstrated to generate significant clinical benefit for treating some cancers in some subjects, many subjects do not clinically respond to such inhibition (Wolchok et al. (2013) N. Engl. J. Med. 369:122-13; Mocellin et al. (2013) Biochim. Hiophys. Aca 1836:187-196; Pardoll et al. (2012) Nit. Rev. Cancer 12:252-264; Brahmer et al. (2012) N. Engl. J. Med. 366:2455-2465; and Topalian et al. (2012) N. Engl. J. Med. 366:2443-2454). For example, only 10-20% of NSCXC patients respond. Accordingly, identifying an accurate biomarker that predicts an effective response has been the subject of intense study. While expression of immune checkpoint inhibitors, such as PD-L1, on tumor cells has been proposed, such expression enriches for response but does not accurately predict sensitivity or responsiveness to anti-immune checkpoint inhibitor therapy. Since therapies that negatively regulate immune checkpoint inhibitors, such as anti-PD-1, anti-PD-L1, and anti-CTLA-4 antibodies, are both significantly toxic in combination and very expensive, there is a great need in the art to identify biomarkers which are predictive of patient responsiveness to such therapies in order to appropriately determine an efficacious and cost-effective course of therapeutic intervention.

### SUMMARY OF THE INVENTION

The present invention is based, at least in part, on the discovery that the presence, amount (e.g., copy number or level of expression) and/or activity of activated Jak kinases are predictive of cancer cell responsiveness to anti-immune checkpoint inhibitor therapies.

In one aspect, a method of determining whether a subject afflicted with a cancer or at risk for developing a cancer would benefit from anti-immune checkpoint inhibitor therapy, the method comprising: a) obtaining a biological sample from the subject; b) determining the presence, copy number, amount, and/or activity of at least one biomarker listed in Table 1 in a subject sample; c) determining the presence, copy number, amount, and/or activity of the at least one biomarker in a control; and d) comparing the presence, copy number, amount, and/or activity of said at least one biomarker detected in steps b) and c), wherein the presence or a significant increase in the copy number, amount, and/or activity of the at least one biomarker in the subject sample relative to the control indicates that the subject afflicted with the cancer or at risk for developing the cancer would benefit from anti-immune checkpoint inhibitor therapy, is provided. In one embodiment, the method further comprises recommending, prescribing, or administering anti-immune checkpoint inhibitor therapy if the cancer is determined to benefit from anti-immune checkpoint inhibitor therapy. In another embodiment, the method further comprises recommending, prescribing, or administering anti-cancer therapy other than anti-immune checkpoint inhibitor therapy if the cancer is determined to not benefit from anti-immune checkpoint inhibitor therapy. In still another embodiment, the anti-cancer therapy is selected from the group consisting of targeted therapy, chemotherapy, radiation therapy, and/or hormonal therapy. In yet another embodiment, the control sample is determined from a cancerous or non-cancerous sample from either the patient or a member of the same species to which the patient belongs. In another embodiment, the control sample comprises cells. In still another embodiment, the method further comprises determining responsiveness to anti-immune checkpoint inhibitor therapy measured by at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

In another aspect, a method of treating a subject afflicted with a cancer, wherein the cancer comprises at least one activating Janus kinase (JAK) mutation shown in Table 1, comprising administering to the subject anti-immune checkpoint inhibitor therapy, thereby treating the subject afflicted with the cancer, is provided. In one embodiment, the at least one activating JAK mutation comprises an activating JAK3 mutation. In another embodiment, the activating JAK3 mutation is a JH2 domain mutation, optionally a JAK3^{V722I} or JAK3^{R657Q} mutation, and/or a FERM domain mutation, optionally a JAK3^{S61C} mutation. In still another embodiment, the method further comprises administering one or more additional anti-cancer agents. In yet another embodiment, the one or more additional anti-cancer agent is a JAK or activator thereof.

In still another aspect, a method of inhibiting hyperproliferative growth of a cancer cell or cells, wherein the cancer cell or cells comprise at least one activating JAK mutation shown in Table 1, comprising contacting the cancer cell or cells with an anti-immune checkpoint inhibitor agent, thereby inhibiting hyperproliferative growth of the cancer cell or cells, is provided. In one embodiment, the step of contacting occurs in vivo, er vivo, or in vitro. In another embodiment, the at least one activating JAK mutation comprises an activating JAK3 mutation. In still another embodiment, the activating JAK3 mutation is a JH2 domain mutation, optionally a JAK3^{V722I} or JAK3^{R657Q} mutation, and/or a FERM domain mutation, optionally a JAK3^{S61C} mutation. In yet another embodiment, the method further comprises administering one or more additional anti-cancer agents. In another embodiment, the one or more additional anti-cancer agent is a JAK or activator thereof.

In yet another aspect, a method of assessing the efficacy of an agent for treating a cancer in a subject, wherein the cancer comprises at least one activating JAK mutation, comprising: a) detecting in a first subject sample and maintained in the presence of the agent the presence, copy number, amount and/or activity of at least one biomarker listed in Table 1; b) detecting the presence, copy number, amount and/or activity of the at least one biomarker listed in Table 1 in a second subject sample and maintained in the absence of the test compound: and c) comparing the presence, copy number, amount and/or activity of the at least one biomarker listed in Table 1 from steps a) and b), wherein the presence or a significantly increased copy number, amount, and/or activity of the at least one biomarker listed in Table 1 in the first subject sample relative to the second subject sample, indicates that the agent treats the cancer in the subject, is provided.

In another aspect, a method of monitoring the progression of a cancer in a subject, wherein the cancer comprises at least one activating JAK mutation, comprising: a) detecting in a subject sample at a first point in time the presence, copy number, amount, and/or activity of at least one biomarker listed in Table 1; b) repeating step a) during at least one subsequent point in time after administration of a therapeutic agent; and c) comparing the presence, copy number, amount, and/or activity detected in steps a) and b), wherein the presence or a significantly increased copy number, amount, and/or activity of the at least one biomarker listed in Table 1 in the first subject sample relative to at least one subsequent subject sample, indicates that the agent treats the cancer in the subject, is provided. In one embodiment, the subject has undergone treatment, completed treatment, and/or is in remission for the cancer in between the first point in time and the subsequent point in time. In another embodiment, the subject has undergone anti-immune checkpoint inhibitor therapy in between the first point in time and the subsequent point in time. In still another embodiment, the first and/or at least one subsequent sample is selected from the group consisting of ex vivo and in vivo samples. In yet another embodiment, the first and/or at least one subsequent sample is obtained from an animal model of the cancer. In another embodiment, the first and/or at least one subsequent sample is a portion of a single sample or pooled samples obtained from the subject.

In still another aspect, a cell-based method for identifying an agent that inhibits a cancer, the method comprising: a) contacting a cell expressing at least one biomarker listed in Table 1 with a test agent; and b) determining the effect of the test agent on the copy number, level of expression, and/or level of activity of the at least one biomarker in Table 1 to thereby identify an agent that inhibits the cancer, is provided. In one embodiment, the method further comprises determining the effect of the test agent on the copy number, level of expression, and/or level of activity of at least one immune checkpoint inhibitor. In another embodiment, said cells are isolated from a source selected from the group consisting of an animal model of a cancer, a subject afflicted with a cancer, and a cell comprising at least one activating JAK3 mutation. In still another embodiment, said cells are unresponsive to anti-immune checkpoint inhibitor therapy. In yet another embodiment, the step of contacting occurs in vivo, er vivo, or in vivo. In another embodiment, the method further comprises determining the ability of the test agent to bind to the at least one biomarker listed in Table 1 before or after determining the effect of the test agent on the copy number, level of expression, or level of activity of the at least one biomarker listed in Table 1.

Numerous embodiments are contemplated for any method, assay, and the like, described herein. For example, in one embodiment, the sample comprises cells, cell lines, histological slides, paraffin embedded tissue, fresh frozen tissue, fresh tissue, biopsies, bronchoalveolar lavage (BAL) fluid, blood, plasma, serum, buccal scrape, saliva, cerebrospinal fluid, urine, stool, mucus, or bone marrow, obtained from the subject. In another embodiment, the presence or copy number is assessed by whole exome sequencing, microarray, quantitative PCR (qPCR), high-throughput sequencing, comparative genomic hybridization (CGH), or fluorescent in sin hybridization (FISH). In still another embodiment, the amount of the at least one biomarker listed in Table 1 is assessed by detecting the presence in the samples of a polynucleotide molecule encoding the biomarker or a portion of said polynucleotide molecule. In yet another embodiment, the polynucleotide molecule is a mRNA, cDNA, or functional variants or fragments thereof. In another embodiment, the step of detecting further comprises amplifying the polynucleotide molecule. In still another embodiment, the amount of the at least one biomarker is assessed by annealing a nucleic acid probe with the sample of the polynucleotide encoding the one or more biomarkers or a portion of said polynucleotide molecule under stringent hybridization conditions. In yet another embodiment, the amount of the at least one biomarker is assessed by detecting the presence a polypeptide of the at least one biomarker. In another embodiment, the presence of said polypeptide is detected using a reagent which specifically binds with said polypeptide. In still another embodiment, the reagent is selected from the group consisting of an antibody, an antibody derivative, and an antibody fragment. In yet another embodiment, the activity of the at least one biomarker is assessed by determining the magnitude of cellular proliferation, cell death, or cytokine production.

In some embodiments, the agent or anti-immune checkpoint inhibitor therapy is selected from the group consisting of a blocking antibody, small molecule, antisense nucleic acid, interfering RNA, shRNA, siRNA, aptamer, ribozyme, dominant-negative protein, and combinations thereof. In another embodiment, the agent is selected from the group consisting of a cytokine, an inhibitor of a Jak kinase inhibitor, a Jak kinase harboring an activating mutation, anti-immune checkpoint inhibitor therapy, and combinations thereof. In still another embodiment, the inhibitor of the Jak kinase inhibitor is an inhibitor of PIAS1, PIAS2, PIAS3, PIAS4, SOCS1, SOCS3, SHP-1, or SHP-2. In yet another embodiment, the agent or anti-immune checkpoint inhibitor therapy is selected from the group consisting of inhibitors of PD-1, PD-L1, PD-L2, CTLA-4, and combinations thereof. In another embodiment, the agent or anti-immune checkpoint inhibitor therapy is a blocking antibody of PD-1, PD-L1, PD-L2, or CTLA-4, and combinations thereof. In still another embodiment, the at least one biomarker is selected from the group consisting of 1, 2, 3, 4, 5, 6, 7, 8, 9, 10, or more biomarkers. In yet another embodiment, the at least one biomarker is an activating JAK3 mutation. In another embodiment, the activating JAK3 mutation is a JH2 domain mutation, optionally a JAK3^{V722I} or JAK3^{R657Q} mutation, and/or a FERM domain mutation, optionally a JAK3^{S61C} mutation. In still another embodiment, the cancer is a solid malignancy. In yet another embodiment, the solid malignancy is selected from the group consisting of lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In another embodiment, the subject is a mammal (e.g., an animal model of cancer, or a human).

### BRIEF DESCRIPTION OF THE DRAWINGS

FIG. 1 includes 4 panels, identified as panels A, B, C, and D, which show long-term durable response to PD-L1 blockade in a patient with metastatic lung adenocarcinoma. Panel A shows systemic therapies received by the patient over time. CT=carboplatin/taxol, CPB=carboplatin/pemetrexedbevacizumab, PB=maintenance pemetrexed/bevacizumab, and PD-L1 inhibitor=MPDL3280A. FIG. 1B shows the size of the left paratracheal mass over time, as measured by longest diameter (cm). Panel B shows the change in patient weight (kg) during the same time period. Panel C shows a chest CT scan prior to initiation of MPDL3280A serial chest CT scans demonstrating reduction in size of the paratracheal mass over time (arrows). Panel D shows serial abdominal CT scans demonstrating recurrence and re-treatment response of the right adrenal mass (arrows).

FIG. 2 includes 4 panels, identified as panels A, B, C, and D, which show that genomic profiling identified two JAK3 alterations present in the tumor that result in constitutive JAK3 activation. Panel A shows structural organization of JAK3 including the N-terminal FERM domain, the SH2 domain, and the JH2 or pseudokinase domain, which is adjacent to the kinase domain and contributes to autoinhibition. Sequencing of position 722 of JAK3 in the JH2 domain reveals heterozygosity for alleles in the germline consistent with a single copy of JAK3^{V722I}, while the left adrenal metastasis revealed loss of heterozygosity (LOH) and complete acquisition of the JAK3^{V722I} allele (predominant band over coverage band). The somatic JAK3^{S61C} mutation was also observed using the Integrated Genomics Viewer (IGV). Panel B shows the results of whole exome sequencing which data revealed apparent copy number neutrality of the JAK3 locus on chromosome 19. Panel C shows the results of that the JAK3^{V722I} allele was detected when analyzed at the allelic level clonality, consistent with the focused sequencing results. Panel D shows an immunoblot of total JAK3, and tyrosine phosphorylated (Y980/981) pJAK3, in 293T cells transfected with EGFP control vector, JAK3^{WT}, JAK3^{S61C}. JAK3^{V722I}, JAK3^{S61C/V722I} or JAK3^{R657Q}.

FIG. 3 includes 2 panels, identified as panels A and B, which show the results of orthogonal sequencing of JAK3 mutations. Polymerase chain reaction (PCR) tracings for V722I (Panel A) and S61C (Panel B) alterations observed in the tumor and germline DNA from the patient are shown.

FIG. 4 shows the copy number profile of the patient's tumor across the exome. The profile is organized by chromosome. CR stands for the copy ratio.

FIG. 5 shows absolute copy number analyses. After correction for tumor purity, ploidy, and allele specific copy number, the absolute copy number derived from ABSOLUTE (Herbst et al. (2014) Nature 515:563-567) is shown by chromosome.

FIG. 6 shows PHIAL results of the patient's somatic exome. Heuristic analysis of the somatic mutations, short insertion/deletions, and copy number alterations across the exome identified 18 mutations for additional evaluation.

FIG. 7 includes 3 panels, identified as panels A, B, and C, which show that deregulated JAK3 signaling induces PD-L1 expression in lung cells. Panel A shows an immunoblot of total JAK3 levels following stable transduction of JAK3^{WT} or the patient derived JAK3^{S61C/V722I} alleles in BEAS-2B or Calu-1 cells. Panel B shows the levels of cell surface PD-L1 expression on these same BEAS-2B or Calu-L cells as measured by flow cytometry using a PD-L1 specific monoclonal antibody compared to isotype. The percent change in isotype-normalized mean fluorescence intensity (MFI) relative to control is highlighted. Panel C shows cell surface PD-L1 expression on Calu-1 cells expressing control vector or the patient derived JAK3^{S61C/V722I} allele, stimulated with or without EGF.

FIG. 8 includes 4 panels, identified as panels A, B, C, and D, which show the results of germline contribution of JAK3^{V722I} to immune cell PD-L1 expression and T cell suppression. Panel A shows the results of PD-L1 and pSTAT3 immunohistochemistry of the patient's adrenal metastasis (arrows denote example tumor cells). Panel B shows levels of tumor cell or immune cell PD-L1 positivity by immunohistochemistry (IHC) across a panel of thoracic malignancies including JAK3^{V722I} and JAK3^{P132T} positive cases or JAK3^{WT} controls (% positive cells and staining intensity, from 0 to 3+, is listed for each tumor and immune cell population from each sample). The case report patient (#4) is marked in bold. Panel C shows PD-L1 MFI on CD14+ myeloid cells from two patients (corresponding to patients 2, 3 and 4 in 3C, denoted with asterisk) or donor PBMCs (n=14) stimulated with IFN-gamma for 48 hours (p=0.02; t-test). Panel D shows the results of blood samples drawn from the index patient immediately pre- and 1 h post-MPDL3280A infusion, and monocytes−/+IFNγ stimulation incubated with T cells from the patient (autologous, pre-MPDL3280A) or a donor (allogeneic). T cell proliferation (frequency of positive cells in gate 4) is shown for autologous or allogeneic CD4+ or CD8+ T cells under each condition.

FIG. 9 includes 2 panels, identified as panels A and B, which show modified H-scores for tumor and immune cells. A comparison of modified H-scores (% positive cells×staining intensity) between V722I-mutant cases and controls for tumor cells (Panel A) and immune cells (Panel B) is shown. P-values were calculated using the Mann-Whitney test.

FIG. 10 shows the results of T cell re-activation following co-culture with JAK3-V722I expressing monocytes in the presence of MPDL3280A. Representative FACS plots of activated autologous CD4 and CD8 T cells (upper panels) or allogeneic CD4 and CD8 T cells (lower panels) following incubation with monocytes primed+/−IFNγ in the absence or presence of MPDL3820A are shown. Highlighted is Gate 4, which was used to quantify the percentage of active T cells.

FIG. 11 shows information on all somatic point mutations and short insertion/deletions observed in the tumor sample from this patient. Additional annotations about protein change, allelic fraction, copy ratio (as segment mean), and other information are provided.

### DETAILED DESCRIPTION OF THE INVENTION

The present invention is based, at least in part, on the discovery that the presence, amount (e.g., copy number or level of expression) and/or activity of activated Jak kinases are predictive of cancer cell responsiveness to anti-immune checkpoint inhibitor therapies. In a retrospective analysis of an exceptional responder to the PD-L1 targeted antibody, MPDL3280a (Genentech), it was determined that the responder had an activating JAK3 V722I mutation. It was further determined that activated Jak kinases (e.g., activating mutations in a Jak kinase itself or biological perturbations resulting in Jak kinase hyperactivity) represent a new mechanism that directly contributes to the induction of the PD-L1 immune checkpoint inhibitor expression in tumors and sensitivity to immune checkpoint blockade. Since activating Jak mutations are only present in 5-10% of cancers and are generally restricted to liquid malignancies, it was surprising to identify Jak mutations as being generally predictive of anti-immune checkpoint inhibitor therapy response and also having such an effect in solid cancers.

Accordingly, the present invention relates, in part, to methods for predicting response of a cancer in a subject to anti-immune checkpoint inhibitor therapy based upon a determination and analysis of specific biomarkers described herein. In addition, such analyses can be used in order to provide useful anti-immune checkpoint inhibitor treatment regimens (e.g., based on predictions of subject survival or relapse, timing of adjuvant or neoadjuvant treatment, etc.).

### I. DEFINITIONS

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples.

The term “altered activity” of a biomarker refers to an activity of the biomarker which is increased or decreased in a disease state, e.g., n a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide, fragment thereof, or biomarker metabolite). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VI-1 and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2: 1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antihodies and Hybridomas 6:93-101) and use of a cysteine residue, biomarker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a biomarker polypeptide or fragment thereof. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the predictive score.”

The term “biomarker” refers to a measurable entity of the present invention that has been determined to be predictive of anti-immune checkpoint inhibitor therapy effects on a cancer. Biomarkers can include, without limitation, nucleic acids, proteins, and metabolites, particularly those shown in Table 1.

For example, “JAKs” are biomarkers of the present invention and refer to a family of non-receptor protein tyrosine kinases known as Janus kinases involved in cytokine receptor signaling. The mammalian JAK protein family consists of four members: JAK1 (Janus kinase-1), JAK2 (Janus kinase-2). JAK3 (also known as Janus kinase leukocyte or JAKL), and TYK2 (protein-tyrosine kinase 2). In some embodiments, JAK1. JAK2, JAK3, TYK2, either alone or in any combination thereof, for use in any aspect of the present invention is contemplated. The JAK kinases mediate the signaling of all receptors belonging to the hematopoietic cytokine receptor type I and type II superfamily and they are required for the biological responses of interferons, most interleukins and colony stimulating factors, and hormones, such as crythropoietin, thrombopoietin, growth hormone, prolactin, and leptin (see, for example, WO 2011/098673; WO 2013/086196; Rawlings et al. (2004) J. Cell Sri. 117:1281-1283). JAK3 in particular selectively binds to receptors and is part of the cytokine signaling pathway for IL-2, IL-4, IL-7, IL-9, IL-15, and IL-21, and modulates IL-10 expression (Yamaoka et al. (2005) 106:3227-3233). JAK1 interacts with, among others, the receptors for cytokines IL-2, IL-4, IL-7, IL-9, and IL-21, while JAK2 interacts with, among others, the receptors for IL-9 and TNFR1 (Pincheira et al. (2008). J. Immunol. 181:1288-1298). Upon binding of certain cytokines to their receptors (for example, IL-2, IL-4, IL-7, IL-9, IL-15, and IL-21), receptor oligomerization occurs, resulting in the cytoplasmic tails of associated JAK kinases being brought into proximity and facilitating the trans-phosphorylation of tyrosine residues on the JAK kinase. This trans-phosphorylation results in the activation of the JAK kinase. Phosphorylated JAK kinases bind various STAT (Signal Transducer and Activator of Transcription) proteins. STAT proteins, which are DNA binding proteins activated by phosphorylation of tyrosine residues, function both as signaling molecules and transcription factors and ultimately bind to specific DNA sequences present in the promoters of cytokine-responsive genes (Darnell (1997) Science 277:1630-1635; Leonard et al. (1998) Ann. Rev Immunol. 16:293-322; Darnell et al. (1994) Science 264:1415-1421). While JAK1, JAK2, and TYK2 are ubiquitously expressed, JAK3 is preferentially expressed in natural killer (NK) cells and not resting T cells, suggesting a role in lymphoid activation (Kawamura et al. (1994) Proc. Natl. Acad. Sci. U.S.A. 91:6374-6378). The results described herein are unexpected given the restricted JAK3 expression pattern. However, JAK3 may also be ectopically expressed in cancer (Verbsky et al. (1996) J. Biol. Chem. 271:13976-13980) and its activity in lung cancer cells is regulated by certain growth factors, such as neuregulin (Liu and Kern (2002) Am. J. Respir. Cell Mol. Biol. 27:306-313). Furthermore, both IL-4 and IL-9 have been shown to signal in lung cancer cells in a JAK3 dependent manner to upregulate the expression of certain cell surface glycoproteins (Damera (2006) Respir Rev 7:39; Damera (2006) Biosci. Rep. 1:55-67), indicating that lung cancer cells can aberrantly engage JAK3-mediated signal transduction, which could influence their behavior.

JAK proteins comprise seven different conserved domains (JAK homology domains, JH1-7) and the structure-function relationships of these domains are well known in the art (see, for example, Rane et al. (2((K)) Oncogene 19:5662-5679; Scott et al. (2002) Clin. Diagn. Lab. Immunol. 9:1153-1159). The carboxyl terminus contains two nearly identical domains, an active kinase domain (JH1) and a catalytically inactive pseudokinase domain (JH2) also termed as a kinase-like domain (KLD). It has been generally acknowledged that JH2 lacks enzymatic activity yet it is involved in regulating the activity of JH1. Both biochemical and cell biological data as well as genetic evidence from human diseases and animal models indicate that JH2 has a dual function in regulation of cytokine signaling. JH2 is required to maintain JAK kinases inactive in the absence of cytokine stimulation, but they are also required for cytokine induced signaling. The region immediately N-terminal to the JH2 is an SH2-like domain consisting of the whole JH3 and a part of JH4. The region immediately N-terminal to the SH2-like domain is a FERM-like domain consisting of a part of JH4 and the whole JH5-JH7. The JAK proteins bind to cytokine receptors through their amino-terminal FERM (Band-4.1, ezrin, radixin, moesin) domains. After the binding of cytokines to their receptors, as stated above, JAKs are activated and phosphorylate the receptors, thereby creating docking sites for signaling molecules, especially for STAT family members (Yamaoka et al. (2004) Genome Biol. 5:253). Like most kinases, JAKs require autophosphorylation for their full activity. In the case of JAK2, the phosphorylation of the activation loop tyrosines 1007 and 1008 are critical for the activity.

Activation of JAK/STAT in cancers may occur by multiple mechanisms including cytokine stimulation (e.g., IL-6 or GM-CSF) or by a reduction in the endogenous suppressors of JAK signaling, such as SOCS (suppressor or cytokinc signaling) or PIAS (protein inhibitor of activated STAT) (Boudny and Kovarik (2002) J. Neoplasm. 49:349-355). Traditionally, JAK inhibition has been desired and it is known, for example, that catalytic inactivation of JH2 domain, such as by an inactivating mutation K581A, K581R or N678A in JH2 of JAK2, abolishes aberrant activation of JAK signaling caused by activating point mutations, such as V617F. In contact, however, it has been determined herein that JAK activation is associated with the upregulation of immune checkpoint inhibitors that render cancer cells more susceptible to anti-immune checkpoint inhibitor therapy.

Mutations in a gene such as a JAK kinase that cause increased activity of the Jak kinase gene or encoded product (e.g., polypeptide, RNA, and the like) are known as “activating mutations.” Such mutations can be constitutive (i.e., always causing increased activity) or transient (e.g., pulsed for a limited duration or inducible). Such mutations can also cause variable increases in JAK activity. Activating mutations are well known in the art for JAKs. For example, point mutations causing constitutively active (i.e., hyper-activating JAK signaling) include, but are not limited to, JAK1-T478S, JAK1-V623A, JAK1-A634D, JAK1-V658F, JAK1-R724H, JAK1-L683, JAK2-V617F, JAK2-M531 I, JAK2-F5371, JAK2-K539L, JAK2-F537-KS39delinsL, JAK2-H538QKS39L, JAK2-H538D+KS39L+1546S. JAK2-H538-K539del, JAK2-D620E, JAK2-V617FD629E. JAK2-V67FC618R, JAK2-V617FC616Y; JAK2-L611 S, JAK2-K607N, JAK2-T875N, JAK3-S61C, JAK3-A572V, JAK3-A573V, JAK3-A593T+A573V, JAK3-V722I, JAK3-P132T or F, TYK2-V678F. and TYK2-P1104A. Other activating JAK mutations are known to a person skilled in the art including, but not limited to, allelic variants, splice variants, derivative variants, substitution variants, deletion variants, and/or insertion variants, fusion polypeptides, orthologs, and interspecies homologs. Any combination of activating JAK mutations is contemplated.

In some embodiments, the term “activating JAK mutations” also encompass biological alterations that result in increased JAK activity. Such biological alterations include, but are not limited to, downregulating or otherwise decreasing or suppressing inhibitors of JAKs, upregulating or otherwise increasing or promoting cytokine signaling through JAKs, and upregulating or otherwise increasing or promoting JAK activity directly or through a direct binding partner in a complex with the JAK. For example, increasing cytokine stimulation (e.g., IL-6 or GM-CSF) or reducing suppressors of JAK signaling, such as SOCS or PIAS.

JAK activity modulators are well known in the art. PIAS proteins, which bind and inhibit at the level of the STAT proteins (Chung et al. (1997) Science 278:1803-1805), are members of an SH2 domain-containing family of proteins able to bind to JAKs and/or receptors and block signaling (see, for example, Aman and Leonard (1997) Curr. Biol. 7:R784-R788; Nicholson and Hilton (1998)J. Leukocyte Biol. 63:665-668). Four members of the PIAS family have been identified, PIAS1, PIAS2 (also known as PIASx), PIAS3, and PIAS4 (also known as PIAS4). PIAS1 was found to bind only to activated Stat1, and PIAS3 to only activated Stat3 (WO 2001/079555; Chung et al. (1997) Science 278:1803-1805; Liu et al. (1998) Proc. Natl. Acad. Sci. U.S.A. 95:10626-10631). PIAS-mediated inhibition of the Jak/Stat signaling pathway, unlike SOCS-mediated inhibition of the Jak/Stat signaling pathway, is very specific.

The SOCS family of proteins have been shown to inhibit the Jak/Stat pathway by inhibiting the activity of the Jaks (Hilton et al. (1998) Proc. Natl. Acad. Sci. U.S.A. 95:114-119; Hilton (1999) Cell. Mol. Life Sci. 55:1658-1677; Trengove and Ward (2013) Am. J. Clin. Exp. Immunol. 2:1-29). The suppressor of cytokine signaling (SOCS) proteins are a family of eight SH2 domain containing proteins which includes the cytokine-inducible SH2 (CIS) domain-containing protein and SOCS-1 to 7. SOCS1 and SOCS3 directly interact with the Jaks and Tyk2 via their kinase inhibitory region (KIR) and SH2 domains, inhibiting the ability of Jak family members to phosphorylate target substrates (Kershaw et al. (2013) Nat. Struct. Mol. Biol. 20:469-476: Babon et al. (2012) Immunity 36:239-250). Once produced, SOCS proteins bind to key components of the signaling apparatus to deactivate and possibly target them for degradation via a conserved C-terminal motif, called the “SOCS Box”, that recruits ubiquitin ligases (see Krebs and Hilton (2000) J. Cell Sci. 113:2813-2819; Yasukawa et al. (2000) Annu. Rev. Immunol. 18:143-164; Greenhalgh and Hilton (2001)J. Leukoc. Biol. 70:348-356). Cytokine-induciblc Src homology 2-containing (CIS) protein, an inhibitor of STAT signaling (Yoshinura et al. (1995) EMBOJ. 14:2816-2826) and CIS-related proteins, which can inhibit STAT signaling and/or directly bind to JAKs, are also SOCS family members (Yoshimura et al. (1995) EMBOJ, 14:2816-2826; Matsumoto et al. (1997) Blood 89:3148-3154; Starr et al. (1997) Nature 387:917-921; Endo et al. (1997) Nature 387:921-924; Naka et al. (1997) Nature 387:924-929) are contemplated. Suppressor of cytokine signaling-1 protein (SOCS-1, also referred to as JAB or SSI-1) associates with all JAKs to block the downstream activation of STAT3 (Ohya et al. (1997), Biol. Chem. 272:27178-27182). SOCS1 expression inhibits IL-6, LIF, oncostatin M, IFN-γ, IFN-β, T-FN-α, thrombopocitin, and growth hormone (GH) induced Jak/Stat signaling. SOCS3 expression inhibits IFN-γ, IFN-β, J-FN-α, GH and leptin. SOCS nucleic acid and polypeptide sequences, such as for SOCS1 and SOCS3, are well known in the art (see, for example, Starr et al. (1997) Nature, 387:917-921; Minamoto et al. (1997) Biochem. Biophyr. Res. Commun. 237:79-83; Masuhara et al. (1997) Biochem. Biophys. Res. Commun. 239:439-446: Naka et al. (1997) Nature 387:924-929; Endo et al. (1997) Nature 387:921-924; WO 1999/028465). Similarly, modulators of SOCS activity are well known in the art (see, for example, U.S. Pat. No. 6,534,277: WO 2004/108955).

SHP-1 and SHP-2 bind to phosphorylated tyrosine residues on receptors or Jaks, and inactivate signaling by dephosphorylating them (Haque et al. (1998). J. Biol. Chem. 273:33898-33896: You et al. (1999) Mol. Cell. Riol. 19:2416-2424). SHP-1, also known as PTPN6, and SHP-2, also known as Syp, SHPTP2, PTP2C, PTPN11, PTP1D, and BPTP3, are members of the family of non-membrane tyrosine phosphatases (U.S. Pat. No. 5,589,375, and U.S. Pat. No. 5,831,009). The SHP proteins contain two src homology 2 (SH2) domains, conserved regions of approximately 100 amino acids originally identified in Src protein tyrosine kinases, that promote protein-protein interactions through phosphotyrosyl residue binding (Neel (1993) Semin. Cell. Biol. 4: 419-432). These two domains have been shown to display differential functions in the regulation of the phosphatase activity and consequently affect different signaling pathways. The N-terminal SH2 domain serves as a regulatory and recruiting domain, producing an autoinhibitory effect through intramolecular interactions with the internal catalytic phosphatase domain. While the C-terminal SH2 domain acts merely to recruit other proteins for intermolecular interactions necessary for signal transduction (Pei et al. (1996) Proc. Natl. Acad. Sci. U.S.A. 93:1141-1145). The phosphorylation state of the SHP molecule regulates its phosphatase activity. Protein-tyrosine phosphatases, including SH2-containing phosphatases, are highly conserved among eukaryotes from such diverse species as mammals, including humans, to yeast and Xenopus. SHP-2 has been shown to play a critical role in aberrant immunological responses (e.g., in the allergic response. (Pazdrak et al. (1997) J. Exp. Med. 186:561-568). SHP phosphorylation is easily detectable by methods known in the art, including, without limitation, the detection of altered mobility of the SHP molecule on a PAGE gel, phosphorylation assays, and assays which measure the activity of the SHP molecule. Detection of SHP phosphorylation may be direct, or alternatively may be indirect, e.g., detection of a downstream activity or event.

Other direct JAK inhibitors, whose elimination promotes JAK activity include tyrophostins, which are derivatives of benzylidene malononitrile, resembling tyrosine and erbstatin moieties (Gazit et al. (1989) J. Med. Chem. 32:2344-2352); AG-490, a member of the tyrophostin family of tyrosine kinase inhibitors (Wang et al. (1999) J. Immunol. 162:3897-3904; Kirken et al. (1999) J. Leukoc. Biol. 65:891-899); 4,5-dimethoxy-2-nitrobenzoic acid and 4,5-dimethoxy-2-nitrobenzamide, which specifically inhibit JAK3 (Goodman et al. (1998) J. Biol. Chem. 273:17742-17748); 4-(phenyl)-amino-6,7-dimethoxyquinazoline (parent compound WH1-258) and derivatives of this compound which are structurally-derived from dimethoxyquinazoline compounds (Sudbeck et al. (1999)); compounds containing a 4′-OH group, including 4-(4′-hydroxyphenyl)-amino-6,7-dimethoxyquinazoline (WH1-P131), 4-(3′-bromo-4′-hydroxylphenyl)-amino-6,7-dimethoxyquinazoline (WH1-P154), and 4-(3′,5′-dibromo-4′-hydroxylphenyl)-amino-6,7-dimethoxyquinazoline (WH1-P97); WH1-P180, another dimethoxyquinazoline compound (Chen et al. (1999) Pharm. Ret. 16:117-122); and cAMP elevating agents, such as forskolin, a direct activator of adenylate cyclase and dibutyryl cAMP, and 3-isobutyl-1-methylxanthine (IBMX), an inhibitor of cAMP phosphodiesterase (Kolenko et al. (1999) Blood 93:2308-2318).

The increases in JAK activity can be measured in any number of ways (e.g., according to measures described herein, including using controls, ratios, comparisons to baselines, and the like). For example, a JAK activating mutation or an activator of JAK activity can enhance the catalytic activity of the JH2 domain or overall JAK activity as compared to the level of such JAK activity in the absence of a stimulator such as a cytokine.

Representative human Jak1 cDNA and protein sequences are well-known in the art and are publicly available from the National Center for Biotechnology Information (NCBI). For example, Jak1 sequences are available under accession numbers NM_002227.2 and NP_002218.2. Nucleic acid and polypeptide sequences of Jak1 orthologs in organisms other than humans are well known and include, for example, chimpanzee Jak1 (XM_001161205.3, XP_001161205.1, XM_001161242.3, and XP_001161242.1), monkey Jak1 (NM_001257909.1 and NP_001244838.1), dog Jak1 (NM_001287126.1 and NP_001274055.1), cow Jak1 (NM_001206534.1 and NP_001193463.1), mouse Jak1 (NM_146145.2 and NP_666257.2), and chicken Jak1 (NM 204870.1 and NP_0.990201.1). Representative Jak1 sequences are presented below in Table 1.

Representative human Jak2 cDNA and protein sequences are well-known in the art and are publicly available from the National Center for Biotechnology Information (NCBI). For example. Jak2 sequences are available under accession numbers NM_004972.3 and NP_004963.1. Nucleic acid and polypeptide sequences of Jak2 orthologs in organisms other than humans are well known and include, for example, chimpanzee Jak2 (XM_003311984.2, XP_003312032.1, XM_001139368.2, and XP 001139368.1), monkey Jak2 (NM_001265901.1 and NP_001252830.1), dog Jak2 (XM_541301.4 and XP_541301.2), mouse Jak2 (NM_008413.3, NP_032439.2, NM_001048177.2, and NP_001041642.1), rat Jak2 (NM_031514.1 and NP_113702.1), and chicken Jak2 (NM_01030538.1 and NP_001025709.1). Representative Jak2 sequences are presented below in Table 1.

Representative human Jak3 cDNA and protein sequences are well-known in the art and are publicly available from the National Center for Biotechnology Information (NCBI). For example, Jak3 sequences are available under accession numbers NM_000215.3 and NP_000206.2. Nucleic acid and polypeptide sequences of Jak3 orthologs in organisms other than humans are well known and include, for example, chimpanzee Jak3 (XM_512502.4 and XP_512502.3), dog Jak3 (XM_005643717.1 and XP_005632774.1), cow Jak3 (XM_002688539.3 and XP_002688585.2), mouse Jak3 (NM_010589.6, NP_034719.2, NM_001190830.1, and NP_001177759.1), rat Jak3 (NM_012855.2 and NP_036987.2), and chicken Jak3 (NM_204996.1 and NP_990327.1). Representative Jak3 sequences are presented below in Table 1.

Representative human Tyk2 cDNA and protein sequences are well-known in the art and are publicly available from the National Center for Biotechnology Information (NCBI). For example, Tyk2 sequences are available under accession numbers NM_0.000215.3 and NP_0.000206.2. Nucleic acid and polypeptide sequences of Tyk2 orthologs in organisms other than humans are well known and include, for example, chimpanzee Tyk2 (XM_001165313.2, XP_001165313.2, XM_003316108.1, and XP_003316156.1), monkey Tyk2 (XM_001101130.2 and XP_001101130.2), dog Tyk2 (XM_005633212.1 and XP_005633269.1), cow Tyk2 (NM_001113764.1 and NP_001107236.1), mouse Tyk2 (NM_018793.2, NP_061263.2, NM_001205312.1, and NP_001192241.1), and rat Tyk2 (NM_1257347.1 and NP_001244276.1). Representative Tyk2 sequences are presented below in Table 1.

Representative human PIAS1 cDNA and protein sequences are well-known in the art and are publicly available from the National Center for Biotechnology Information (NCBI). For example, PIAS1 sequences are available under accession numbers NM_016166.1 and NP_057250.1. Nucleic acid and polypeptide sequences of PIAS1 orthologs in organisms other than humans are well known and include, for example, monkey PIAS1 (NM_001266301.2 and NP_001253230.1), cow PIAS1 (NM_1075396.2 and NP_001068864.1), mouse PIAS1 (NM_019663.3 and NP_062637.2), rat PIAS1 (NM_001106829.2 and NP_001100299.2), and chicken PIAS1 (NM_001031456.1 and NP_001026627.1). Representative PIAS1 sequences are presented below in Table 1.

Representative human PIAS2 cDNA and protein sequences are well-known in the art and are publicly available from the National Center for Biotechnology Information (NCBI). For example, human PIAS2 isoform 1 is available under accession numbers NM_173206.3 and NP_775298.1. The transcript variant uses an alternate 3′ coding exon compared to variant 2 resulting in a shorter isoform that has a unique C-terminus relative to isoform 2.

Human PIAS2 isoform 2, available under accession numbers NM_004671.3 and NP_004662.2 represents the longer transcript and encodes the longer isoform. Nucleic acid and polypeptide sequences of PIAS2 orthologs in organisms other than humans are well known and include, for example, chimpanzee PIAS2 (XM_001147441.3, XP_001147441.2, XM_003953281.1, and XP_00395330.1), monkey PIAS2 (XM_001085456.2 and XP_001085456.2), mouse PIAS2 (NM_008602.4, NP_032628.3, NM_001164170.1, NP_001157642.1, NM_001164169.1, NP_001157641.1, NM_001164168.1, NP_001157640.1, NM_001164167.1, and NP_001157639.1), rat PIAS2 (NM_053337.1 and NP_445789.1), and chicken PIAS2 (NM_001030626.1 and NP_001025797.1). Representative PIAS2 sequences are presented below in Table 1.

Representative human PIAS3 cDNA and protein sequences are well-known in the art and are publicly available from the National Center for Biotechnology Information (NCBI). For example, PIAS3 sequences are available under accession numbers NM_006099.3 and NP_006090.2. Nucleic acid and polypeptide sequences of PIAS3 orthologs in organisms other than humans are well known and include, for example, chimpanzee PIAS3 (XM_003949491.1 and XP_003949540.1), monkey PIAS3 (XM_001095153.2 and XP_001095153.2), cow PIAS3 (NM_001102185.1 and NP_001095655.1), mouse PIAS3 (NM_146135.2, NP_0.666247.1, NM_018812.2, NP_061282.2, NM_001165949.1, and NP_001159421.1), and rat PIAS3 (NM_031784.2 and NP_113972.2). Representative PIAS3 sequences are presented below in Table 1.

Representative human PIAS4 cDNA and protein sequences are well-known in the art and are publicly available from the National Center for Biotechnology Information (NCBI). For example, PIAS4 sequences are available under accession numbers NM_015897.2 and NP_056981.2. Nucleic acid and polypeptide sequences of PIAS4 orthologs in organisms other than humans are well known and include, for example, dog PIAS4 (XM_542167.5 and XP_542167.4), cow PIAS4 (NM_001083482.2 and NP_001076951.1), mouse PIAS4 (NM_021501.4 and NP_067476.2), and rat PIAS4 (NM_1100757.1 and NP_001094227.1). Representative PIAS4 sequences are presented below in Table 1.

Representative human SOCS1 cDNA and protein sequences are well-known in the art and are publicly available from the National Center for Biotechnology Information (NCBI). For example, SOCS1 sequences are available under accession numbers NM_003745.1 and NP_003736.1. Nucleic acid and polypeptide sequences of SOCS1 orthologs in organisms other than humans are well known and include, for example, chimpanzee SOCS1 (XM_001141793.3 and XP_001141793.1), monkey SOCS1 (XM_001104595.2 and XP_001104595.1), dog SOCS1 (XM_005622079.1 and XP_005622136.1), cow SOCS1 (XM_002697964.2 and XP_002698010.1), mouse SOCS1 (NM_009896.2, NP_034026.1, NM_001271603.1, and NP_001258532.1), rat SOCS1 (NM_145879.2 and NP_665886.2), and chicken SOCS1 (NM_001137648.1 and NP_001131120.1). Representative SOCS1 sequences are presented below in Table 1.

Representative human SOCS3 cDNA and protein sequences are well-known in the art and are publicly available from the National Center for Biotechnology Information (NCBI). For example, SOCS1 sequences are available under accession numbers NM_003955.4 and NP_003946.3. Nucleic acid and polypeptide sequences of SOCS3 orthologs in organisms other than humans are well known and include, for example, chimpanzee SOCS3 (XM_001157032.3 and XP_001157032.1), monkey SOCS3 (NM_001194326.1 and NP_001181255.1), dog SOCS3 (NM_001031631.1 and NP_001026801.1), cow SOCS3 (NM_174466.2 and NP_776891.1), mouse SOCS3 (NM_07707.3 and NP_031733.1), rat SOCS3 (NM_053565, and NP_446017.1), and chicken SOCS3 (NM_204600.1 and NP_989931.1). Representative SOCS3 sequences are presented below in Table 1.

Nucleic acid and polypeptide sequences of other SOCS orthologs in organisms, including humans, are also well known. For example, nucleic acid and polypeptide sequences of cytokine-inducible S1H2 (CIS) are well known and include, for example, human CIS (NM_145071.2. NP_659508.1, NM_013324.5, and NP_037456.5), chimpanzee CIS (XM_526202.3, XP_526202.3, XM_003309810.1, and XP_003309858.1), monkey CIS (NM_001258075.1 and NP_001245004.1), dog CIS (XM_541873.4 and XP_5541873.3), cow CIS (NM_0.001046586.1 and NP_001040051.1), mouse CIS (NM_009895.3 and NP_034025.1), rat CIS (NM_031804.1 and NP_113992.1), and chicken CIS (NM_204626.1 and NP_989957.1). Nucleic acid and polypeptide sequences of SOCS2 are well known and include, for example, human SOCS2 (NM_003877.4, NP_003868.1, NM_0012704710.1, NM_001257400.1, NM_001270470.1, NM_001257399.1, NM_001270469.1, NM_001257398.1, NM_001270468.1, NM_001257397.1, NM_001270467.1, and NM_001257396.1), chimpanzee SOCS2 (XM_001139989.3 and XP_001139989.1), monkey SOCS2 (NM_001194762.1 and NP_001181691.1), cow SOCS2 (NM_177523.2 and NP_803489.1), mouse SOCS2 (NM_007706.4, NP_031732.1, NM_001168657.1, NP_001162128.1, NM_001168656.1, NP_00162127.1, NM_00168655.1, and NP_001162126.1), rat SOCS2 (NM_058208.1 and NP_478115.1), and chicken SOCS2 (NM_204540.1 and NP_89871.1). Nucleic acid and polypeptide sequences of SOCS4 are well known and include for example, human SOCS4 (NM_199421.1, NP_955453.1, NM_080867.2, and NP_543143.1), monkey SOCS4 (NM_001193820.1 and NP_001180749.1), dog SOCS4 (XM_003435136.3 and XP_003435184.1), cow SOCS4 (NM_001076218.2 and NP_001069686.1), mouse SOCS4 (NM_080843.2 and NP_543119.2), rat SOCS4 (NM_001107256.2 and NP_001100726.1), and chicken SOCS4 (NM_001199108.1 and NP_001186037.1). Nucleic acid and polypeptide sequences of SOCS5 are well known and include for example, human SOCS5 (NM_144949.2, NP_659198.1, NM_014011.4, and NP_0.054730.1), chimpanzee SOCS5 (XM_515453.3 and XP_515453.2), monkey SOCS5 (NM_001266928.1 and NP_001253857.1), cow SOCS5 (XM_005626083.1 and XP_005626140.1), cow SOCS5 (NM_001046182.1 and NP_001039647.1), mouse SOCS5 (XM_006524675.1, XP_006524738.1, XM_006524671.1, XP_006524734.1, XM_006524672.1, XP_006524735.1, XM_006524673.1, XP_006524736.1, XM_6524674.1, and XP_006524737.1), rat SOCS5 (NM_001109274.1 and NP_001102744.1), and chicken SOCS5 (NM_001127314.1 and NP_001120786.1). Nucleic acid and polypeptide sequences of SOCS6 are well known and include for example, human SOCS6 (NM_004232.3 and NP_004223.2), mouse SOCS6 (NM_018821.4 and NP_061291.2), rat SOCS6 (NM_001271149.1 and NP_001258078.1), and chicken SOCS6 (NM_001127312.1 and NP_001120784.1). Finally, nucleic acid and polypeptide sequences of SOCS7 are well known and include for example, human SOCS7 (NM_014598.3 and NP_055413.1), chimpanzee SOCS7 (XM_003954433.1 and XP_003954482.1), monkey SOCS7 (XM_001082440.2 and XP_001082440.2), dog SOCS7 (XM_005624981.1 and XP_005625038.1), mouse SOCS7 (NM_138657.3 and NP_619598.1), and rat SOCS7 (XM_006247484.1 and XP_006247546.1).

Representative human SHP-1 cDNA and protein sequences are well-known in the art and are publicly available from the National Center for Biotechnology Information (NCBJ). For example, SHP-1 isoform 1 is available under accession numbers NM_002831.5 and NP_002822.2. Transcript variant 1 encoding isoform 1 represents the predominant transcript and encodes the shortest isoform. Transcript variant 2 (NM_080548.4) uses an alternate 5′ terminal exon compared to transcript variant 1 resulting in a SHP-1 isoform 2 (NP_536858.1) with a distinct and 2 amino acid longer N-terminus as compared to isoform 1. Finally, transcript variant 3 (NM_0800549.3) uses an alternate 5′ terminal exon and an alternate acceptor splice site at the penultimate exon as compared to transcript variant 1 resulting in a longer isoform (SHP-1 isoform 3; NP_536859.1; also known as SHP-1L) with a distinct N- and C-terminus as compared to isoform 1. Nucleic acid and polypeptide sequences of SHP-1 orthologs in organisms other than humans are well known and include, for example, monkey SHP-1 (XM_001110915.2 and XP_001110915.1), dog SHP-1 (XM_005637211.1 and XP_005637268.1), cow SHP-1 (NM_001098017.1 and NP_001091486.1), mouse SHP-1 (NM_013545.3, NP_038573.2, NM_001077705.2, and NP_001071173.1), rat SHP-1 (NM_053908.1 and NP_446360.1), and chicken SHP-1 (NM_001031484.1 and NP_001026655.1). Representative SHP-1 sequences are presented below in Table 1.

Representative human SHP-2 cDNA and protein sequences are well-known in the art and are publicly available from the National Center for Biotechnology Information (NCBI). For example, SHP-2 isoform 1 is available under accession numbers NM_002834.3 and NP_002825.3. Transcript variant 1 encoding isoform 1 represents the longer transcript and encodes the longer isoform. Transcript variant 2 (NM_080601.1) differs in the 3′ untranslated region (UTR) and coding sequence as compared to transcript variant 1 resulting in a SHP-2 isoform 2 (NP_542168.1) with a shorter and distinct N-terminus as compared to isoform 1. Nucleic acid and polypeptide sequences of SHP-2 orthologs in organisms other than humans are well known and include, for example, chimpanzee SHP-2 (XM_522535.4 and XP_522535.3), monkey SHP-2 (NM_001261109.1 and NP_001248038.1), dog SHP-2 (XM_005636251.1, XP_005636308.1, XM_005636250.1, and XP_005636307.1), cow SHP-2 (XM_002694590.3 and XP_002694636.2), mouse SHP-2 (NM_011202.3, NP_035332.1, NM_001109992.1, and NP_001103462.1), rat SHP-2 (NM_013088.2, NP_037220.2, NM_001177593.1, and NP_001171064.1), and chicken SHP-2 (NM_204968.1 and NP_90299.1). Representative SHP-2 sequences are presented below in Table 1.

It is to be noted that the biomarkers described herein can be used to refer to any combination of features described herein regarding any individual or combination of such biomarkers. For example, any combination of sequence composition, percentage identity, sequence length, domain structure, Functional activity, mutation status, etc. can be used to describe a biomarker molecule of the present invention.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluid that are normally not (e.g., bronchoalveolar lavage fluid, amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of immune checkpoint inhibitors, such as PD-1, PD-L1, PD-L2, and/or CTLA-4. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaccous gland carcinoma, papillary carcinoma, papillary adenocarcinomas, cystadenocarcinoma, medullary carcinoma, bronchogenic carcinoma, renal cell carcinoma, hcpatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myclocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell. Brenner, or undifferentiated.

In some embodiments, lung cancer subtypes are included. For example, according to the American Cancer Society, there are two major types of lung cancer: small cell lung cancer (SCLC) and non-small cell lung cancer (NSCLC). SCLC comprises about 15% of all cancers. NSCLC, however, comprises about 85% of all lung cancers and is divided into three distinct sub-types: squamous cell carcinoma (about 25-30% of the cases), large cell carcinomas (about 10-15%), and adenocarcinomas (about 40%). The cells in these sub-types differ in size, shape, and chemical make-up. These lung cancers are inclusive of bronchogenic carcinoma, bronchial carcinoids, chondromatous hamartoma, solitary pulmonary nodules, pulmonary sarcomas, undifferentiated small cell carcinoma, undifferentiated large cell carcinoma, and broncholoalvcolar carcinomas. Each such lung cancer subtype is contemplated for use according to the present invention, either alone or in any combination.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid, protein, or metabolite is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is determining whether to provide targeted therapy against a cancer to provide immunotherapy that generally increases immune responses against the cancer (e.g., anti-immune checkpoint inhibitor therapy). Another example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of two or more coordinately expressed biomarkers. For example, the genes, proteins, metabolites, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, cosinophils, mast cells, basophils, and granulocytes.

The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well known in the art and include, without limitation, CTLA-4. PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fe portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or in combination, are used to inhibit immune checkpoint inhibitors.

“PD-1” is an immune checkpoint inhibitor that refers to a member of the immunoglobulin gene superfamily that functions as a coinhibitory receptor having PD-L1 and PD-L2 as known ligands. PD-1 was previously identified using a subtraction cloning based approach to select for proteins involved in apoptotic cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520). These features also define a larger family of polypeptides, called the immunoinhibitory receptors, which also includes gp49B, PIR-B, and the killer inhibitory receptors (KIRs) (Vivier and Dacron (1997) Immunol. Today 18:286). It is often assumed that the tyrosyl phosphorylated ITIM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 206):285-8). Nucleic acid and polypeptide sequences of PD-1 orthologs in organisms other than humans are well known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, 87-2, PD-1 ligand, and/or other polypeptides on antigen presenting cells.

The term “PD-1 activity” includes the ability of a PD-1 polypeptide to modulate an inhibitory signal in an activated immune cell, e.g., by engaging a natural PD-1 ligand on an antigen presenting cell. PD-1 transmits an inhibitory signal to an immune cell in a manner similar to CTLA4. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) J. Exp. Med. 192:1027 for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see, Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1 (CD80), B7-2 (CD86), inducible costimulatory ligand (ICOS-L). B7-H3, B7-H4, VISTA, B7-H6, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (see the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two (sheets, each consisting of anti-parallel 0 strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

The term “PD-L1” refers to a specific PD-1 ligand. Two forms of human PD-L1 molecules have been identified. One form is a naturally occurring PD-L1 soluble polypeptide, i.e., having a short hydrophilic domain at the COOH-terminal end and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. The signal sequence is from about amino acid 1 to about amino acid 18. The signal sequence is from about amino acid 1 to about amino acid 18. The IgV domain is from about amino acid 19 to about amino acid 134 and the IgV domain is from about amino acid 19 to about amino acid 134. The IgC domain is from about amino acid 135 to about amino acid 227 and the IgC domain of SEQ ID NO: 6 is shown from about amino acid 135 to about amino acid 227. The hydrophilic tail of PD-L1 comprises a hydrophilic tail shown from about amino acid 228 to about amino acid 245. The PD-L polypeptide comprises a transmembrane domain shown from about amino acids 239 to about amino acid 259 and a cytoplasmic domain shown of about 30 amino acids from 260 to about amino acid 290. In addition, nucleic acid and polypeptide sequences of PD-L1 orthologs in organisms other than humans are well known and include, for example, mouse PD-L1 (NM_021893.3 and NP_068693.1), rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 comprise a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosincs, or tryptophans. Transmembrane domains are described in, for example, Zagotta et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-12 polypeptide and amino acid residues 102-200 of the mature polypeptide comprise an IgC domain. As used herein, IgV and IgC (domains are recognized in the art as Ig superfamily member domains. These domains correspond to structural units that have distinct folding patterns called Ig folds. Ig folds are comprised of a sandwich of two 6 sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the C1 set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well known and include, for example, mouse PD-L2 (NM_021396.2 and NP_067371.1), rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-12 polypeptide with its natural binding partner, e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g. T lymphocytes, and 4) modulate the immune response of an organism, e.g., a mouse or human organism.

The term “immune response” includes T cell-mediated and/or B cell-mediated immune responses. Exemplary immune responses include T cell responses, e.g., cytokinc production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, I.e., culture medium represents less than about 20%, more preferably less than about 10%, and most preferably less than about 5% of the volume of the protein preparation.

A “kit” is any manufacture (e.g. a package or container) comprising at least one reagent, e.g. a probe or small molecule, for specifically detecting and/or affecting the expression of a marker of the invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allow patients with large breast cancer to undergo breast-conserving surgery.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, truncation variants, and/or insertion variants, fusion polypeptides, orthologs, and/or interspecies homologs. By way of example, at least one mutation of a Jak protein would include a Jak protein in which part of all of the sequence of a polypeptide or gene encoding the Jak protein is absent or not expressed in the cell for at least one Jak protein produced in the cell. For example, a Jak protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a Jak protein would include a Jak protein having one or more amino acid differences in its amino acid sequence compared with wild type of the same Jak protein. By way of another example, a mutated Jak3 polypeptide is a Jak3 polypeptide having at least one amino acid difference compared to wild type Jak3 polypeptide. Mutations may be somatic and/or germline.

An “over-expression” or “significantly higher level of expression” of a biomarker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “predictive” includes the use of a biomarker nucleic acid, protein, and/or metabolite status, e.g., over- or under-activity, emergence, expression, growth, remission, recurrence or resistance of tumors before, during or after therapy, for determining the likelihood of response of a cancer to anti-immune checkpoint inhibitor treatment (e.g., therapeutic antibodies against PD-1, PD-L1, PD-L2, and/or CTLA-4). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by IHC, Northern Blot, or qPCR), increased or decreased biomarker protein (e.g., by IHC) and/or biomarker metabolite, or increased or decreased activity (determined by, for example, modulation of biomarkers, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular anti-immune checkpoint inhibitor therapy or those developing resistance thereto).

The terms “prevent,” “preventing.” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as lung cancer, melanoma, and renal cell carcinoma), development of one or more clinical factors, development of intestinal cancer, or recovery from the disease.

The term “response to anti-immune checkpoint inhibitor therapy” relates to any response of the hyperproliferative disorder (e.g., cancer) to an anti-immune checkpoint inhibitor therapy, such as anti-immune checkpoint inhibitor therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” in some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the invention, or a fragment thereof, short interfering RNA (siRNA), and small molecules which interfere with or inhibit expression of a target biomarker nucleic acid by RNA interference (RNAi).

“RNA interference (RNAi)” is an evolutionary conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs, siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint inhibitor, chemotherapeutic, and/or radiation therapy). In some embodiments, normal cells are not affected to an extent that causes the normal cells to be unduly injured by the anti-immune checkpoint inhibitor therapy. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L. Baker J A, Moran E M., Cancer Res 1984; 94: 161-173: Weisenthal L M, Lippman M E, Cancer Treat Rep 1985; 69: 615-632; Weisenthal L M. In: Kaspers G J L, Pieters R, Twentyman P R. Weisenthal L M. Veerman A J P, eds. Drug Resistance in Leukemia and Lymphoma. Langhorne, P A: Harwood Academic Publishers, 1993: 415-432; Weisenthal L M, Contrib Gynecol Obstet 1994. 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 41%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “synergistic effect” refers to the combined effect of two or more anti-immune checkpoint inhibitor agents can be greater than the sum of the separate effects of the anticancer agents alone.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40) nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 94):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “subject” refers to any healthy animal, mammal or human, or any animal, mammal or human afflicted with a cancer, e.g., lung, ovarian, pancreatic, liver, breast, prostate, and colon carcinomas, as well as melanoma and multiple myeloma. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED). Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 6000%, 700%, 800%, 900%, 100×% or more increased for the agent relative to no administration of the agent. Also, Similarly, the IC₅₀(i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynuclecotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid | GAC, GAT |
| (Asp, D) | |
| Cysteine (Cys, C) | TGC, TGT |
| Glutamic acid | GAA, GAG |
| (Glu, E) | |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine | TTC, TTT |
| (Phe, F) | |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Table 1) are well known in the art and readily available on publicly available databases, such as the National Center for Biotechnology Information (NCBI). For example, exemplary nucleic acid and amino acid sequences derived from publicly available sequence databases are provided below.

TABLE 1
SEQ ID NO: 1 Human Jak1 cDNA sequence

| | |
|---|---|
| 1 | atgcagtatc taaatataaa agaggactgc aatgccatgg ctttctgtgc taaaatgagg |
| 61 | agctccaaga agactgaggt gaacctggag gcccctgagc caggggtgga agtgatcttc |
| l21 | tatctgtcgg acagggagcc cctccggctg ggcagtggag agtacacagc agaggaactg |
| 181 | tgcatcaggg ctgcacaggc atgccgtatc tctcctcttt gtcacaacct ctttgccctg |
| 241 | tatgacgaga acaccaagct ctggtatgct ccaaatcgca ccatcaccgt tgatgacaag |
| 301 | atgtccctcc ggctccacta ccggatgagg ttctatttca ccaattggca tggaaccaac |
| 361 | gacaatgagc agtcagtgtg gcgtcattct ccaaagaagc agaaaaatgg ctacgagaaa |
| 421 | aaaaagattc cagatgcaac ccctctcctt gatgccagct cactggagta tctgtttgct |
| 481 | cagggacagt atgatttggt gaaatgcctg gctcctattc gagaccccaa gaccgagcag |
| 541 | gatggacatg atattgagaa cgagtgtcta gggatggctg tcctggccat ctcacactat |
| 601 | gccatgatga agaagatgca gttgccagaa ctgcccaagg acatcagcta caagcgatat |
| 661 | attccagaaa cattgaataa gtccatcaga cagaggaacc ttctcaccag gatgcggata |
| 721 | aataatgttt tcaaggattt cctaaaggaa tttaacaaca agaccatttg tgacagcagc |
| 781 | gtgtccacgc atgacctgaa ggtgaaatac ttggctacct tggaaacttt gacaaaacat |
| 841 | tacggtgctg aaatatttga gacttccatg ttactgattt cattagaaaa tgagatgaat |
| 901 | tggtttcatt cgaatgacgg tggaaacgtt ctctactacg aagtgatggt gactgggaat |
| 961 | cttggaatcc agtggaggca taaaccaaat gttgtttctg ttgaaaagga aaaaaataaa |
| 1021 | ctgaagcgga aaaaactgga aaataaacac aagaaggatg aggagaaaaa caagatccgg |
| 1081 | gaagagtgga acaatttttc ttacttccct gaaatcactc acattgtaat aaaggagtct |
| 1141 | gtggtcagca ttaacaagca ggacaacaag aaaatggaac tgaagctctc ttcccacgag |
| 1201 | gaggccttgt cctttgtgtc cctggtagat ggctacttcc ggcttacagc agatgcccat |
| 1261 | cattacctct gcaccgacgt ggcccccccg ttgatcgtcc acaacataca gaatggctgt |
| 1321 | catggtccaa tctgtacaga atacgccatc aataaattgc ggcaagaagg aagcgaggag |
| 1381 | gggatgtacg tgctgaggtg gagctgcacc gactttgaca acatcctcat gaccgtcacc |
| 1441 | tgctttgaga agtctgagca ggtgcagggt gcccagaagc agttcaagaa ctttcagatc |
| 1501 | gaggtgcaga agggccgcta cagtctgcac ggttcggacc gcagcttccc cagcttggga |
| 1561 | gacctcatga gccacctcaa gaagcagatc ctgcgcacgg ataacatcag cttcatgcta |
| 1621 | aaacgctgct gccagcccaa gccccgagaa atctccaacc tgctggtggc tactaagaaa |
| 1681 | gcccaggagt ggcagcccgt ctaccccatg agccagctga gtttcgatcg gatcctcaag |
| 1741 | aaggatctgg tgcagggcga gcaccttggg agaggcacga gaacacacat ctattctggg |
| 1801 | accctgatgg attacaagga tgacgaagga acttctgaag agaagaagat aaaagtgatc |
| 1861 | ctcaaagtct tagaccccag ccacagggat atttccctgg ccttcttcga ggcagccagc |
| 1921 | atgatgagac aggtctccca caaacacatc gtgtacctct atggcgtctg tgtccgcgac |
| 1981 | gtggagaata tcatggtgga agagtttgtg gaagggggtc ctctggatct cttcatgcac |
| 2041 | cggaaaagcg atgtccttac cacaccatgg aaattcaaag ttgccaaaca gctggccagt |
| 2101 | gccctgagct acttggagga taaagacctg gtccatggaa atgtgtgtac taaaaacctc |
| 2161 | ctcctggccc gtgagggcat cgacagtgag tgtggcccat tcatcaagct cagtgacccc |
| 2221 | ggcatcccca ttacggtgct gtctaggcaa gaatgcattg aacgaatccc atggattgct |
| 2281 | cctgagtgtg ttgaggactc caagaacctg agtgtggctg ctgacaagtg gagctttgga |
| 2341 | accacgctct gggaaatctg ctacaatggc gagatcccct tgaaagacaa gacgctgatt |
| 2401 | gagaaagaga gattctatga aagccggtgc aggccagtga caccatcatg taaggagctg |
| 2461 | gctgacctca tgacccgctg catgaactat gaccccaatc agaggccttt cttccgagcc |
| 2521 | atcatgagag acattaataa gcttgaagag cagaatccag atattgtttc agaaaaaaaa |
| 2581 | ccagcaactg aagtggaccc cacacatttt gaaaagcgct tcctaaagag gatccgtgac |
| 2641 | ttgggagagg gccactttgg gaaggttgag ctctgcaggt atgaccccga aggggacaat |
| 2701 | acaggggagc aggtggctgt taaatctctg aagcctgaga gtggaggtaa ccacatagct |
| 2761 | gatctgaaaa aggaaatcga gatcttaagg aacctctatc atgagaacat tgtgaagtac |
| 2821 | aaaggaatct gcacagaaga cggaggaaat ggtattaagc tcatcatgga atttctgcct |
| 2881 | tcgggaagcc ttaaggaata tcttccaaag aataagaaca aaataaacct caaacagcag |
| 2941 | ctaaaatatg ccgttcagat ttgtaagggg atggactatt tgggttctcg gcaataggtt |
| 3001 | caccgggact tggcagcaag aaatgtcctt gttgagagtg aacaccaagt gaaaattgga |
| 3061 | gacttcggtt taaccaaagc aattgaaacc gataaggagt attacaccgt caaggatgac |
| 3121 | cgggacagcc ctgtgttttg gtatgctcca gaatgtttaa tgcaatctaa attttatatt |
| 3181 | gcctctgacg tctggtcttt tggagtcact ctgcatgagc tgctgactta ctgtgattca |
| 3241 | gattctagtc ccatggcttt gttcctgaaa atgataggcc caacccatgg ccagatgaca |
| 3301 | gtcacaagac ttgtgaatac gttaaaagaa ggaaaacgcc tgccgtgccc acctaactgt |
| 3361 | ccagatgagg tttatcaact tatgaggaaa tgctgggaat tccaaccatc caatcggaca |
| 3421 | agctttcaga accttattga aggatttgaa gcacttttaa aataa |

SEQ ID NO: 2 Human Jak1 amino acid sequence

| | |
|---|---|
| 1 | mgylnikedc namafcakmr sskktevnle apepgvevif ylsdreplrl vsgeytaeel |
| 61 | ciraaqacri splchnlfal ydentklwya pnrtitvddk mslrlhyrmr fyftnwhgtn |
| 121 | dneqsvwrhs pkkqkngyek kkipdatpll dassleylfa qgqydlvkcl apirdpkteq |
| 181 | dghdienecl gmavlaishy ammkkmqlpe lpkdisykry ipetlnksir qrnlltrmri |
| 241 | nnvfkdflke fnnkticdss vsthdlkvky latletltkh ygaeifetsm llissenemn |
| 301 | wfhsndggnv lyyevmvtgn lgiqwrhkpn vvsvekeknk lkrkklenkh kkdeeknkir |
| 361 | eewnnfsyfp eithivikes vvsinkqdnk kmelklsshe ealsfvs1vd gyfrltadah |
| 421 | hylctdvapp livhniqngc hgpicteyai nklrqegsee gmyvlrwsct dfdnilmtvt |
| 481 | cfekseqvqg aqkqfknfqi evqkgryslh gsdrsfpslg dlmshlkkqi lrtdnisfml |
| 541 | krccqpkpre isnllvatkk aqewqpvypm sqlsfdrilk kdlvqgehlg rgtrthiysg |
| 601 | tlmdylddeg tseekkikvi lkvldpshrd islaffeaas mmrqvshkhi vylygvcvrd |
| 661 | venimveefv eggpldlfmh rksdvlttpw kfkvakqlas alsyledkdl vhgnvctknl |
| 721 | llaregidse cgpfiklsdp gipitvlsrq ecieripwia pecvedsknl svaadkwsfg |
| 781 | ttlweicyng edplkdktli ekerfyesrc rpvtpsckel adlmtrcmny dpnqrpffra |
| 841 | imrdinklee qnpdivsekk patevdpthf ekrflkrird lgeghfgkve lcrydpegdn |
| 901 | tgeqvavksl kpesggnhia dlkkeieilr nlyhenivky kgictedggn giklimeflp |
| 961 | sgslkeylpk nknkinlkqq lkyavqickg mdylgsrgyv hrdlaarnvl vesehqvkig |
| 1021 | dfgltkaiet dkeyytvkdd rdspvfwyap eclmqskfyi asdvwsfgvt lhelltycds |
| 1081 | dsspmalflk migpthgqmt vtrlvntlke gkrlpcppnc pdevyqlmrk cwefqpsnrt |
| 1141 | sfqnliegfe allk |

SEQ ID NO: 3 Human Jak2 cDNA sequence

| | |
|---|---|
| 1 | atgggaatgg cctgccttac gatgacagaa atggagggaa catccacctc ttctatatat |
| 61 | cagaatggtg atatttctgg aaatgccaat tctatgaagc aaatagatcc agttcttcag |
| 121 | gtgtatcttt accattccct tgggaaatct gaggcagatt atctgacctt tccatctggg |
| 181 | gagtatgttg cagaagaaat ctgtattgct gcttctaaag cttgtggtat cacacctgtg |
| 241 | tatcataata tgtttgcttt aatgagtgaa acagaaagga tctggtatcc acccaaccat |
| 301 | gtcttccata tagatgagtc aaccaggcat aatgtactct acagaataag attttacttt |
| 361 | cctcgttggt attgcagtgg cagcaacaga gcctatcggc atggaatatc tcgaggtgct |
| 421 | gaagctcctc ttcttgatga ctttgtcatg tcttacctct ttgctcagtg gcggcatgat |
| 481 | tttgtgcacg gatggataaa agtacctgtg actcatgaaa cacaggaaga atgtcttggg |
| 541 | atggcagtgt tagatatgat gagaatagcc aaagaaaacg atcaaacccc actggccatc |
| 601 | tataactcta tcagctacaa gacattctta ccaaaatgta ttcgagcaaa gatccaagac |
| 661 | tatcatattt tgacaaggaa gcgaataagg tacagatttc gcagatttat tcagcaattc |
| 721 | agccaatgca aagccactgc cagaaacttg aaacttaagt atcttataaa tctggaaact |
| 781 | ctgcagtctg ccttctacac agagaaattt gaagtaaaag aacctggaag tggtccttca |
| 841 | ggtgaggaga tttttgcaac cattataata actggaaacg gtggaattca gtggtcaaga |
| 901 | gggaaacata aagaaagtga gacactgaca gaacaggatt tacagttata ttgcgatttt |
| 961 | cctaatatta ttgatgtcag tattaagcaa gcaaaccaag agggttcaaa tgaaagccga |
| 1021 | gttgtaacta tccataagca agatggtaaa aatctggaaa ttgaacttag ctcattaagg |
| 1081 | gaagctttgt ctttcgtgtc attaattgat ggatattata gattaactgc agatgcacat |
| 1141 | cattacctct gtaaagaagt agcacctcca gccgtgcttg aaaatataca aagcaactgt |
| 1201 | catggcccaa tttcgatgga ttttgccatt agtaaactga agaaagcagg taatcagact |
| 1261 | ggactgtatg tacttcgatg cagtcctaag gactttaata aatatttttt gacttttgct |
| 1321 | gtcgagcgag aaaatgtcat tgaatataaa cactgtttga ttacaaaaaa tgagaatgaa |
| 1381 | gagtacaacc tcagtgggac aaagaagaac ttcagcagtc ttaaagatct tttgaattgt |
| 1441 | taccagatgg aaactgttcg ctcagacaat ataattttcc agtttactaa atgctgtccc |
| 1501 | ccaaagccaa aagataaatc aaaccttcta gtcttcagaa cgaatggtgt ttctgatgta |
| 1561 | ccaacctcac caacattaca gaggcctact catatgaacc aaatggtgtt tcacaaaatc |
| 1621 | agaaatgaag atttgatatt taatgaaagc cttggccaag gcacttttac aaagattttt |
| 1681 | aaaggcgtac gaagagaagt aggagactac ggtcaactgc atgaaacaga agttctttta |
| 1741 | aaagttctgg ataaagcaca cagaaactat tcagagtctt tctttgaagc agcaagtatg |
| 1801 | atgagcaagc tttctcacaa gcatttggtt ttaaattatg gagtatgtgt ctgtggagac |
| 1861 | gagaatattc tggttcagga gtttgtaaaa tttggatcac tagatacata tctgaaaaag |
| 1921 | aataaaaatt gtataaatat attatggaaa cttgaagttg ctaaacagtt ggcatgggcc |
| 1981 | atgcattttc tagaagaaaa cacccttatt catgggaatg tatgtgccaa aaatattctg |
| 2041 | cttatcagag aagaagacag gaagacagga aatcctcctt tcatcaaact tagtgatcct |
| 2101 | ggcattagta ttacagtttt gccaaaggac attcttcagg agagaatacc atgggtacca |
| 2161 | cctgaatgca ttgaaaatcc taaaaattta aatttggcaa cagacaaatg gagttttggt |
| 2221 | accactttgt gggaaatctg cagtggagga gataaacctc taagtgctct ggattctcaa |
| 2281 | agaaagctac aattttatga agataggcat cagcttcctg caccaaagtg ggcagaatta |
| 2341 | gcaaacctta taaataattg tatggattat gaaccagatt tcaggccttc tttcagagcc |
| 2401 | atcatacgag atcttaacag tttgtttact ccagattatg aactattaac agaccatgac |
| 2461 | atgttaccaa atatgaggat aggtgccctg gggttttctg gtgcctttga agaccgggat |
| 2521 | cctacacagt ttgaagagag acatttgaaa tttctacagc aacttggcaa gggtaatttt |
| 2581 | gggagtgtgg agatgtgccg gtatgaccct ctacaggaca acactgggga ggtggtcgct |
| 2641 | gtaaaaaagc ttcagcatag tactgaagag cacctaagag actttgaaag ggaaattgaa |
| 2701 | atcttgaaat ccctacagca tgacaacatt gtaaagtaca agggagtgtg ctacagtgct |
| 2761 | ggtcggcgta atctaaaatt aattatggaa tatttaccat atggaagttt acgagactat |
| 2821 | cttcaaaaac ataaagaacg gatagatcac ataaaacttc tgcagtacac atctcagata |
| 2881 | tgcaagggta tggagtatct tggtacaaaa aggtatatcc acagggatct ggcaacgaga |
| 2941 | aatatattgg tggagaacga gaacagagtt aaaattggag attttgggtt aaccaaagtc |
| 3001 | ttgccacaag acaaagaata ctataaagta aaagaacctg gtgaaagtcc catattctgg |
| 3061 | tatgctccag aatcactgac agagagcaag ttttctgtgg cctcagatgt ttggagcttt |
| 3121 | ggagtggttc tgtatgaact tttcacatac attgagaaga gtaaaagtcc accagcggaa |
| 3181 | tttatgcgta tgattggcaa tgacaaacaa ggacagatga tcgtgttcca tttgatagaa |
| 3241 | cttttgaaga ataatggaag attaccaaga ccagatggat gcccagatga gatctatatg |
| 3301 | atcatgacag aatgctggaa caataatgta aatcaacgcc cctcctttag ggatctagct |
| 3361 | cttcgagtgg atcaaataag ggataacatg gctggatga |

SEQ ID NO: 4 Human Jak2 amino acid sequence

| | |
|---|---|
| 1 | mgmacltmte megtstssiy qngdisgnan smkqidpvlq vylyhslgks eadyltfpsg |
| 61 | eyvaeeicia askacgitpv yhnmfalmse teriwyppnh vfhidestrh nvlyrirfyf |
| 121 | prwycsgsnr ayrhgisrga eapllddfvm sylfaqwrhd fvhgwikvpv thetqeeclg |
| 181 | mavldmmria kendqtplai ynsisyktfl pkcirakiqd yhiltrkrir yrfrrfiqqf |
| 241 | sqckatarnl klkylinlet lqsafytekf evkepgsgps geeifatiii tgnggiqwsr |
| 301 | gkhkesetlt eqdlqlycdf pniidvsikq anqegsnesr vvtihkqdgk nleielsslr |
| 361 | ealsfvslid gyyrltadah hylckevapp avleniqsnc hgpismdfai sklkkagnqt |
| 421 | glyvlrcspk dfnkyfltfa verenvieyk hclitknene eynlsgtkkn fsslkdllnc |
| 481 | yqmetvrsdn iifqftkccp pkpkdksnll vfrtngvsdv ptsptlqrpt hmnqmvfhki |
| 541 | rnedlifnes egqgtftkif kgvrrevgdy gqlgetevll kvldkahrny sesffeaasm |
| 601 | msklshkhlv lnygvcvcgd enilvqefvk fgsldtylkk nkncinilwk levakqlawa |
| 661 | mhfleevtli hgnvcaknil lireedrktg nppfiklsdp gisitvlpkd ilqeripwvp |
| 721 | pecienpknl nlatdkwsfg ttlweicsgg dkplsaldsq rklqfyedrh qlpapkwael |
| 781 | anlinncmdy eqdfrpsfra iirdlnslft pdyelltend mlpnmrigal gfsgafedrd |
| 841 | ptqfeerhlk flqqlgkgnf gsvemcrydp lqdntgevva vkklqhstee hlrdfereie |
| 901 | ilkslqhdni vkykgvcysa grrnlklime ylpygslrdy lqkhkeridh ikllqytsqi |
| 961 | ckgmeylgtk ryihrdlatr nilvenenrv kigdfgltkv lpqdkeyykv kepgespifw |
| 1021 | yapesltesk fsvasdvwsf gvvlyetfty ieksksppae fmrmigndkq gqmivfhlie |
| 1081 | liknngrlpr pdgcpdeiym imtecwnnnv nqrpsfrdla lrvdqirdnm ag |

SEQ ID NO: 5 Human Jak3 cDNA sequence

| | |
|---|---|
| 1 | atggcacctc caagtgaaga gacgcccctg atccctcagc gttcatgcag cctcttgtcc |
| 61 | acggaggctg gtgccctgca tgtgctgctg cccgctcggg gccccgggcc cccccagcgc |
| 121 | ctatctttct cctttgggga ccacttggct gaggacctgt gcgtgcaggc tgccaaggcc |
| 181 | agcggcatcc tgcctgtgta ccactccctc tttgctctgg ccacggagga cctgtcctgc |
| 241 | tggttccccc cgagccacat cttctccgtg gaggatgcca gcacccaagt cctgctgtac |
| 301 | aggattcgct tttacttccc caattggttt gggctggaga agtgccaccg cttcgggcta |
| 361 | cgcaaggatt tggccagtgc tatccttgac ctgccagtcc tggagcacct ctttgcccag |
| 421 | caccgcagtg acctggtgag tgggcgcctc cccgtgggcc tcagtctcaa ggagcagggt |
| 481 | gagtgtctca gcctggccgt gttggacctg gcccggatgg cgcgagagca ggcccagcgg |
| 541 | ccgggagagc tgctgaagac tgtcagctac aaggcctgcc tacccccaag cctgcgcgac |
| 601 | ctgatccagg gcctgagctt cgtgacgcgg aggcgtattc ggaggacggt gcgcagagcc |
| 661 | ctgcgccgcg tggccgcctg ccaggcagac cggcactcgc tcatggccaa gtacatcatg |
| 721 | gacctggagc ggctggatcc agccggggcc gccgagacct tccacgtggg cctccctggg |
| 781 | gcccttggtg gccacgacgg gctggggctg ctccgcgtgg ctggtgacgg cggcatcgcc |
| 841 | tggacccagg gagaacagga ggtcctccag cccttctgcg actttccaga aatcgtagac |
| 901 | attagcatca agcaggcccc gcgcgttggc ccggccggag agcaccgcct ggtcactgtt |
| 961 | accaggacag acaaccagat tttagaggcc gagttcccag ggctgcccga ggctctgtcg |
| 1021 | ttcgtggcgc tcgtggacgg ctacttccgg ctgaccacgg actcccagca cttcttctgc |
| 1081 | aaggaggtgg caccgccgag gctgctggag gaagtggccg agcagtgcca cggccccatc |
| 1141 | actctggact ttgccatcaa caagctcaag actgggggct cacgtcctgg ctcctatgtt |
| 1201 | ctccgccgca gcccccagga ctttgacagc ttcctcctca ctgtctgtgt ccagaacccc |
| 1261 | cttggtcctg attataaggg ctgcctcatc cggcgcagcc ccacaggaac cttccttctg |
| 1321 | gttggcctca gccgacccca cagcagtctt cgagagctcc tggcaacctg ctgggatggg |
| 1381 | gggctgcacg tagatggggt ggcagtgacc ctcacttcct gctgtatccc cagacccaaa |
| 1441 | gaaaagtcca acctgatcgt ggtccagaga ggtcacagcc cacccacatc atccttggtt |
| 1501 | cagccccaat cccaatacca gctgagtcag atgacatttc acaagatccc tgctgacagc |
| 1561 | ctggagtggc atgagaacct gggccatggg tccttcacca agatttaccg gggctgtcgc |
| 1621 | catgaggtgg tggatgggga ggcccgaaag acagaggtgc tgctgaaggt catggatgcc |
| 1681 | aagcacaaga actgcatgga gtcattcctg gaagcagcga gcttgatgag ccaagtgtcg |
| 1741 | taccggcatc tcgtgctgct ccacggcgtg tgcatggctg gagacagcac catggtgcag |
| 1801 | gaatttgtac acctgggggc catagacatg tatctgcgaa aacgtggcca cctggtgcca |
| 1861 | gccagctgga agctgcaggt ggtcaaacag ctggcctacg ccctcaacta tctggaggac |
| 1921 | aaaggcctgc cccatggcaa tgtctctgcc cggaaggtgc tcctggctcg ggagggggct |
| 1981 | gatgggagcc cgcccttcat caagctgagt gaccctgggg tcagccccgc tgtgttaagc |
| 2041 | ctggagatgc tcaccgacag gatcccctgg gtggcccccg agtgtctccg ggaggcgcag |
| 2101 | acacttagct tggaagctga caagtggggc ttcggcgcca cggtctggga agtgtttagt |
| 2161 | ggcgtcacca tgcccatcag tgccctggat cctgctaaga aactccaatt ttatgaggac |
| 2221 | cggcagcagc tgccggcccc caagtggaca gagctggccc tgctgattca acagtgcatg |
| 2281 | gcctatgagc cggtccagag gccctccttc cgagccgtca ttcgtgacct caatagcctc |
| 2341 | atctcttcag actatgagct cctctcagac cccacacctg gtgccctggc acctcgtgat |
| 2401 | gggctgtgga atggtgccca gctctatgcc tgccaagacc ccacgatctt cgaggagaga |
| 2461 | cacctcaagt acatctcaca gctgggcaag ggcaactttg gcagcgtgga gctgtgccgc |
| 2521 | tatgacccgc taggcgacaa tacaggtgcc ctggtggccg tgaaacagct gcagcacagc |
| 2581 | gggccagacc agcagaggga ctttcagcgg gagattcaga tcctcaaagc actgcacagt |
| 2641 | gatttcattg tcaagtatcg tggtgtcagc tatggcccgg gccgccagag cctgcggctg |
| 2701 | gtcatggagt acctgcccag cggctgcttg cgcgacttcc tgcagcggca ccgcgcgcgc |
| 2761 | ctcgatgcca gccgcctcct tctctattcc tcgcagatct gcaagggcat ggagtacctg |
| 2821 | ggctcccgcc gctgcgtgca ccgcgacctg gccgcccgaa acatcctcgt ggagagcgag |
| 2881 | gcacacgtca agatcgctga cttcggccta gctaagctgc tgccgcttga caaagactac |
| 2941 | tacgtggtcc gcgagccagg ccagagcccc attttctggt atgcccccga atccctctcg |
| 3001 | gacaacatct tctctcgcca gtcagacgtc tggagcttcg gggtcgtcct gtacgagctc |
| 3061 | ttcacctact gcgacaaaag ctgcagcccc tcggccgagt tcctgcggat gatgggatgt |
| 3121 | gagcgggatg tccccgccct ctgccgcctc ttggaactgc tggaggaggg ccagaggctg |
| 3181 | ccggcgcctc ctgcctgccc tgctgaggtt cacgagctca tgaagctgtg ctgggcccct |
| 3241 | agcccacagg accggccatc attcagcgcc ctgggccccc agctggacat gctgtggagc |
| 3301 | ggaagccggg ggtgtgagac tcatgccttc actgctcacc cagagggcaa acaccactcc |
| 3361 | ctgtcctttt catag |

SEQ ID NO: 6 Human Jak3 amino acid sequence

| | |
|---|---|
| 1 | mappseetpl ipqrscslls teagalhvll pargpgppqr lsfsfgdhla edlcvqaaka |
| 61 | sgilpvyhsl falatedlsc wfppshifsv edastqvlly rirfyfpnwf glekchrfgl |
| 121 | rkdlasaild lpvlehlfaq hrsdlvsgrl pvglslkeqg eclslavldl armareqaqr |
| 181 | pgellktvsy kaclppslrd liqglsfvtr rrirrtvrra lrrvaacqad rhslmakyim |
| 241 | dlerldpaga aetfhvglpg algghdglgl lrvagdggia wtqgeqevlq pfcdfpeivd |
| 301 | isikqaprvg pagehrlvtv trtdnqilea efpglpeals fvalvdgyfr lttdsqhffc |
| 361 | kevapprlle evaeqchgpi tldfainklk tggsrpgsyv lrrspqdfds flltvcvqnp |
| 421 | lgpdykgcli rrsptgtfll vglsrphssl rellatcwdg glhvdgvavt ltscciprpk |
| 481 | eksnlivvqr ghspptsslv qpqsqyqlsq mtfhkipads lewhenlghg sftkiyrgcr |
| 541 | hevvdgeark tevllkvmda khkncmesfl eaaslmsqvs yrhlvllhgv cmagdstmvq |
| 601 | efvhlgaidm ylrkrghlvp aswklqvvkq layalnyled kglphgnvsa rkvllarega |
| 661 | dgsppfikls dpgvspavls lemltdripw vapeclreaq tlsleadkwg fgatvwevfs |
| 721 | gvtmpisald pakklqfyed rqqlpapkwt elalliqqcm ayepvqrpsf ravirdlnsl |
| 781 | issdyellsd ptpgalaprd glwngaqlya cqdptifeer hlkyisqlgk gnfgsvelcr |
| 841 | ydplgdntga lvavkqlqhs gpdqqrdfqr eiqilkalhs dfivkyrgvs ygpgrqslrl |
| 901 | vmeylpsgcl rdflqrhrar ldasrlllys sqickgmeyl gsrrcvhrdl aarnilvese |
| 961 | ahvkiadfgl akllpldkdy yvvrepgqsp ifwyapesls dnifsrqsdv wsfgvvlyel |
| 1021 | ftycdkscsp saeflrmmgc erdvpalcrl lelleegqrl pappacpaev helmklcwap |
| 1081 | spqdrpsfsa lgpqldmlws gsrgcethaf tahpegkhhs lsfs |

SEQ ID NO: 7 Human Tyk2 cDNA sequence

| | |
|---|---|
| 1 | atgcctctgc gccactgggg gatggccagg ggcagtaagc ccgttgggga tggagcccag |
| 61 | cccatggctg ccatgggagg cctgaaggtg cttctgcact gggctggtcc aggcggcggg |
| 121 | gagccctggg tcactttcag tgagtcatcg ctgacagctg aggaagtctg catccacatt |
| 181 | gcacataaag ttggtatcac tcctccttgc ttcaatctct ttgccctctt cgatgctcag |
| 241 | gcccaagtct ggttgccccc aaaccacatc ctagagatcc ccagagatgc aagcctgatg |
| 301 | ctatatttcc gcataaggtt ttatttccgg aactggcatg gcatgaatcc tcgggaaccg |
| 361 | gctgtgtacc gttgtgggcc cccaggaacc gaggcatcct cagatcagac agcacagggg |
| 421 | atgcaactcc tggacccagc ctcatttgag tacctctttg agcagggcaa gcatgagttt |
| 481 | gtgaatgacg tggcatcact gtgggagctg tcgaccgagg aggagatcca ccactttaag |
| 541 | aatgagagcc tgggcatggc ctttctgcac ctctgtcacc tcgctctccg ccatggcatc |
| 601 | cccctggagg aggtggccaa gaagaccagc ttcaaggact gcatcccgcg ctccttccgc |
| 661 | cggcatatcc ggcagcacag cgccctgacc cggctgcgcc ttcggaacgt cttccgcagg |
| 721 | ttcctgcggg acttccagcc gggccgactc tcccagcaga tggtcatggt caaataccta |
| 781 | gccacactcg agcggctggc accccgcttc ggcacagagc gtgtgcccgt gtgccacctg |
| 841 | aggctgctgg cccaggccga gggggagccc tgctacatcc gggacagtgg ggtggcccct |
| 901 | acagaccctg gccctgagtc tgctgctggg cccccaaccc acgaggtgct ggtgacaggc |
| 961 | actggtggca tccagtggtg gccagtagag gaggaggtga acaaggagga gggttctagt |
| 1021 | ggcagcagtg gcaggaaccc ccaagccagc ctgtttggga agaaggccaa ggctcacaag |
| 1081 | gcagtcggcc agccggcaga caggccgcgg gagccactgt gggcctactt ctgtgacttc |
| 1141 | cgggacatca cccacgtggt gctgaaagag cactgtgtca gcatccaccg gcaggacaac |
| 1201 | aagtgcctgg agctgagctt gccttcccgg gctgcggcgc tgtccttcgt gtcgctggtg |
| 1261 | gacggctatt tccgcctgac ggccgactcc agccactacc tgtgccacga ggtggctccc |
| 1321 | ccacggctgg tgatgagcat ccgggatggg atccacggac ccctgctgga gccatttgtg |
| 1381 | caggccaagc tgcggcccga ggacggcctg tacctcattc actggagcac cagccacccc |
| 1441 | taccgcctga tcctcacagt ggcccagcgt agccaggcac cagacggcat gcagagcttg |
| 1501 | cggctccgaa agttccccat tgagcagcag gacggggcct tcgtgctgga gggctggggc |
| 1561 | cggtccttcc ccagcgttcg ggaacttggg gctgccttgc agggctgctt gctgagggcc |
| 1621 | ggggatgact gcttctctct gcgtcgctgt tgcctgcccc aaccaggaga aacctccaat |
| 1681 | ctcatcatca tgcggggggc tcgggccagc cccaggacac tcaacctcag ccagctcagc |
| 1741 | ttccaccggg ttgaccagaa ggagatcacc cagctgtccc acttgggcca gggcacaagg |
| 1801 | accaacgtgt atgagggccg cctgcgagtg gagggcagcg gggaccctga ggagggcaag |
| 1861 | atggatgacg aggaccccct cgtgcctggc agggaccgtg ggcaggagct acgagtggtg |
| 1921 | ctcaaagtgc tggaccctag tcaccatgac atcgccctgg ccttctacga gacagccagc |
| 1981 | ctcatgagcc aggtctccca cacgcacctg gccttcgtgc atggcgtctg tgtgcgcggc |
| 2041 | cctgaaaata tcatggtgac agagtacgtg gagcacggac ccctggatgt gtggctgcgg |
| 2101 | agggagcggg gccatgtgcc catggcttgg aagatggtgg tggcccagca gctggccagc |
| 2161 | gccctcagct acctggagaa caagaacctg gttcatggta atgtgtgtgg ccggaacatc |
| 2221 | ctgctggccc ggctggggtt ggcagagggc accagcccct tcatcaagct gagtgatcct |
| 2281 | ggcgtgggcc tgggcgccct ctccagggag gagcgggtgg agaggatccc ctggctggcc |
| 2341 | cccgaatgcc taccaggtgg ggccaacagc ctaagcaccg ccatggacaa gtgggggttt |
| 2401 | ggcgccaccc tcctggagat ctgctttgac ggagaggccc ctctgcagag ccgcagtccc |
| 2461 | tccgagaagg agcatttcta ccagaggcag caccggctgc ccgagccctc ctgcccacag |
| 2521 | ctggccacac tcaccagcca gtgtctgacc tatgagccaa cccagaggcc atcattccgc |
| 2581 | accatcctgc gtgacctcac ccggctgcag ccccacaatc ttgctgacgt cttgactgtg |
| 2641 | aacccggact caccggcgtc ggaccctacg gttttccaca agcgctattt gaaaaagatc |
| 2701 | cgagatctgg gcgagggtca cttcggcaag gtcagcttgt actgctacga tccgaccaac |
| 2761 | gacggcactg gcgagatggt ggcggtgaaa gccctcaagg cagactgcgg cccccagcac |
| 2821 | cgctcgggct ggaagcagga gattgacatt ctgcgcacgc tctaccacga gcacatcatc |
| 2881 | aagtacaagg gctgctgcga ggaccaaggc gagaagtcgc tgcagctggt catggagtac |
| 2941 | gtgcccctgg gcagcctccg agactacctg ccccggcaca gcatcgggct ggcccagctg |
| 3001 | ctgctcttcg cccagcagat ctgcgagggc atggcctatc tgcacgcgca gcactacatc |
| 3061 | caccgagacc tagccgcgcg caacgtgctg ctggacaacg acaggctggt caagatcggg |
| 3121 | gactttggcc tagccaaggc cgtgcccgaa ggccacgagt actaccgcgt gcgcgaggat |
| 3181 | ggggacagcc ccgtgttctg gtatgcccca gagtgcctga aggagtataa gttctactat |
| 3241 | gcgtcagatg tctggtcctt cggggtgacc ctgtatgagc tgctgacgca ctgtgactcc |
| 3301 | agccagagcc cccccacgaa attccttgag ctcataggca ttgctcaggg tcagatgaca |
| 3361 | gttctgagac tcactgagtt gctggaacga ggggagaggc tgccacggcc cgacaaatgt |
| 3421 | ccctgtgagg tctatcatct catgaagaac tgctgggaga cagaggcgtc ctttcgccca |
| 3481 | accttcgaga acctcatacc cattctgaag acagtccatg agaagtacca aggccaggcc |
| 3541 | ccttcagtgt tcagcgtgtg ctga |

SEQ ID NO: 8 Human Tyk2 amino acid sequence

| | |
|---|---|
| 1 | mplrhwgmar gskpvgdgaq pmaamgglkv llhwagpggg epwvtfsess ltaeevcihi |
| 61 | ahkvgitppc fnlfalfdaq aqvwlppnhi leiprdaslm lyfrirfyfr nwhgmnprep |
| 121 | avyrcgppgt eassdqtaqg mqlldpasfe ylfeqgkhef vndvaslwel steeeihhfk |
| 181 | neslgmaflh lchlalrhgi pleevakkts fkdciprsfr rhirqhsalt rlrlrnvfrr |
| 241 | flrdfqpgrl sqqmvmvkyl atlerlaprf gtervpvchl rllaqaegep cyirdsgvap |
| 301 | tdpgpesaag ppthevlvtg tggiqwwpve eevnkeegss gssgrnpqas lfgkkakahk |
| 361 | avgqpadrpr eplwayrcdf rdithvvlke hcvsihrqdn kclelslpsr aaalsfvslv |
| 421 | dgyfrltads shylchevap prlvmsirdg ihgpllepfv qaklrpedgl ylihwstshp |
| 481 | yrliltvaqr sqapdgmqsl rlrkfpieqq dgafvlegwg rsfpsvrelg aalqgcllra |
| 541 | gddcfslrrc clpqpgetsn liimrgaras prtlnlsqls fhrvdqkeit qlshlgqgtr |
| 601 | tnvyegrlrv egsgdpeegk mddedplvpg rdrgqelrvv lkvldpshhd ialafyetas |
| 661 | lmsqvshthl afvhgvcvrg penimvteyv ehgpldvwlr rerghvpmaw kmvvaqqlas |
| 721 | alsylenknl vhgnvcgrni llarlglaeg tspfiklsdp gvglgalsre erveripwla |
| 781 | peclpggans lstamdkwgf gatlleicfd geaplqsrsp sekehfyqrq hrlpepscpq |
| 841 | latltsqclt yeptqrpsfr tilrdltrlq phnladvltv npdspasdpt vfhkrylkki |
| 901 | rdlgeghfgk vslycydptn dgtgemvavk alkadcgpqh rsgwkqeidi lrtlyhehii |
| 961 | kykgccedqg ekslqlvmey vplgslrdyl prhsiglaql llfaqqiceg maylhaqhyi |
| 1021 | hrdlaarnvl ldndrlvkig dfglakavpe gheyyrvred gdspvfwyap eclkeykfyy |
| 1081 | asdvwsfgvt lyellthcds sqspptkfle ligiaqgqmt vlrlteller gerlprpdkc |
| 1141 | pcevyhlmkn cweteasfrp tfenlipilk tvhekyqgqa psvfsvc |

SEQ ID NO: 9 Human PIAS1 cDNA sequence

| | |
|---|---|
| 1 | atggcggaca gtgcggaact aaagcaaatg gttatgagcc ttagagtttc tgaactccaa |
| 61 | gtactgttgg gctacgccgg gagaaacaag cacggacgca aacacgaact tctcacaaaa |
| 121 | gccctgcatt tgctaaaggc tggctgtagt cctgctgtgc aaatgaaaat taaggaactc |
| 181 | tataggcggc ggttcccaca gaaaatcatg acgcctgcag acttgtccat ccccaacgta |
| 241 | cattcaagtc ctatgccagc aactttgtct ccatctacca ttccacaact cacttacgat |
| 301 | ggtcaccctg catcatcgcc attactccct gtttctcttc tgggacctaa acatgaactg |
| 361 | gaactcccac atcttacatc agctcttcac ccagtccatc cggatataaa acttcaaaaa |
| 421 | ttaccatttt atgatttact ggatgaactg ataaaaccca ccagtctagc atcagacaac |
| 481 | agtcagcgct ttcgagaaac ctgttttgca tttgccttga caccacaaca agtgcagcaa |
| 541 | atcagtagtt ccatggatat ttctgggacc aaatgtgact tcacagtaca ggtccagtta |
| 601 | aggttttgtt tatcagaaac cagttgtcca caagaagatc acttcccacc caatctttgt |
| 661 | gtgaaagtga atacaaaacc ttgcagcctt ccaggttacc ttccacctac aaaaaatggc |
| 721 | gtggaaccaa agcgacccag ccgaccaatt aatatcacct cacttgtccg actgtccaca |
| 781 | acagtaccaa acacgattgt tgtttcttgg actgcagaaa ttggaagaaa ctattccatg |
| 841 | gcagtatatc ttgtaaaaca gttgtcctca acagttcttc ttcagaggtt acgagcaaag |
| 901 | ggaataagga atccggatca ttctagagct ttaattaaag agaagttgac tgcggatcca |
| 961 | gacagtgaaa tagctacaac cagcctaagg gtttctctac tatgtccact tggtaaaatg |
| 1021 | cggctgacaa ttccgtgtcg ggcccttaca tgttctcatc tacaatgttt tgacgcaact |
| 1081 | ctttacattc agatgaatga gaaaaaacca acctgggttt gtcctgtctg tgataagaag |
| 1141 | gctccatatg aacaccttat tattgatggc ttgtttatgg aaatcctaaa gtactgtaca |
| 1201 | gactgtgatg aaatacaatt taaggaggat ggcacttggg caccgatgag atcaaaaaag |
| 1261 | gaagtacagg aagtttctgc ctcttacaat ggagtcgatg gatgcttgag ctccacattg |
| 1321 | gagcatcagg tagcgtctca ccaccagtcc tcaaataaaa acaagaaagt agaagtgatt |
| 1381 | gacctaacca tagacagttc atctgatgaa gaggaagaag agccatctgc caagaggacc |
| 1441 | tgtccttccc tatctcccac atcaccacta aataataaag gcattttaag tcttccacat |
| 1501 | caagcatctc cagtatcccg caccccaagc cttcctgctg tagacacaag ctacattaat |
| 1561 | acctccctca tccaagacta taggcatcct ttccacatga cacccatgcc ttacgactta |
| 1621 | caaggattag atttctttcc tttcttatca ggagacaatc agcattacaa cacctccttg |
| 1681 | cttgccgctg cagcagcagc agtttcagat gatcaagacc tcctacactc gtctcggttt |
| 1741 | ttcccgtata cctcctcaca gatgtttctt gatcagttaa gtgcaggagg cagtacttct |
| 1801 | ctgccaacca ccaatggaag cagtagtggc agtaacagca gcctggtttc ttccaacagc |
| 1861 | ctaagggaaa gccatagcca caccgtcaca aacaggagca gcacggacac ggcatccatc |
| 1921 | tttggcatca taccagacat tatttcattg gactga |

SEQ ID NO: 10 Human PIAS1 amino acid seqence

| | |
|---|---|
| 1 | madsaelkqm vmslrvselq vllgyagrnk hgrkhelltk alhllkagcs pavqmkikel |
| 61 | yrrrfpqkim tpadlsipnv hsspmpatls pstipqltyd ghpasspllp vsllgpkhel |
| 121 | elphltsalh pvhpdiklqk lpfydlldel ikptslasdn sqrfretcfa faltpqqvqq |
| 181 | isssmdisgt kcdftvqvql rfclsetscp qedhfppnlc vkvntkpcsl pgylpptkng |
| 241 | vepkrpsrpi nitslvrlst tvpntivvsw taeigrnysm avylvkqlss tvllqrlrak |
| 301 | girnpdhsra likekltadp dseiattslr vsllcplgkm rltipcralt cshlqcfdat |
| 361 | lyiqmnekkp twvcpvcdkk apyehliidg lfmeilkyct dcdeiqfked gtwapmrskk |
| 421 | evqevsasyn gvdgclsstl ehqvashhqs snknkkvevi dltidsssde eeeepsakrt |
| 481 | cpslsptspl nnkgilslph qaspvsrtps lpavdtsyin tsliqdyrhp fhmtpmpydl |
| 541 | qgldffpfls gdnqhyntsl laaaaaavsd dqdllhssrf fpytssqmfl dqlsaggsts |
| 601 | lpttngsssg snsslvssns lreshshtvt nrsstdtasi fgiipdiisl d |

SEQ ID NO: 11 Human PIAS2 (transcript variant 1) cDNA sequence

| | |
|---|---|
| 1 | atggcggatt tcgaagagtt gaggaatatg gtttctagtt ttagggtttc tgaactacaa |
| 61 | gtattactag gctttgctgg acggaataaa agtggacgca agcatgacct cctgatgagg |
| 121 | gcgctgcatt tattgaagag cggctgcagc cctgcggttc agattaaaat ccgagaattg |
| 181 | tatagacgcc gatatccacg aactcttgaa ggactttctg atttatccac aatcaaatca |
| 241 | tcggttttca gtttggatgg tggctcatca cctgtagaac ctgacttggc cgtggctgga |
| 301 | atccactcgt tgccttccac ttcagttaca cctcactcac catcctctcc tgttggttct |
| 361 | gtgctgcttc aagatactaa gcccacattt gagatgcagc agccatctcc cccaattcct |
| 421 | cctgtccatc ctgatgtgca gttaaaaaat ctgccctttt atgatgtcct tgatgttctc |
| 481 | atcaagccca cgagtttagt tcaaagcagt attcagcgat ttcaagagaa gttttttatt |
| 541 | tttgctttga cacctcaaca agttagagag atatgcatat ccagggattt tttgccaggt |
| 601 | ggtaggagag attatacagt ccaagttcag ttgagacttt gcctggcaga gacaagttgc |
| 661 | cctcaagaag ataactatcc aaatagtcta tgtataaaag taaatgggaa gctatttcct |
| 721 | ttgcctggct atgcaccacc gcctaaaaat gggattgaac agaagcgccc tggacgcccc |
| 781 | ttgaatatta catctttagt taggttatct tcagctgtgc caaaccaaat ttccatttct |
| 841 | tgggcatcag aaattgggaa gaattactct atgtctgtat atcttgtacg gcagcttaca |
| 901 | tcagccatgt tattacagag attaaaaatg aaaggtatta gaaaccctga tcattccaga |
| 961 | gcactaatta aagaaaaact tactgcagat cctgatagtg aaattgctac aactagcctt |
| 1021 | cgggtatcct tgatgtgccc tttaggaaaa atgaggctga caatcccatg ccgtgcagtg |
| 1081 | acttgtacac atctgcagtg ttttgatgct gccctctatc tacaaatgaa tgagaaaaag |
| 1141 | cccacctgga tttgtcctgt gtgtgacaaa aaagctgcct atgaaagtct aatattagat |
| 1201 | gggcttttta tggaaattct caatgactgt tctgatgtag atgagatcaa attccaagaa |
| 1261 | gatggttctt ggtgtccaat gagaccgaag aaagaagcta tgaaagtatc cagccaaccg |
| 1321 | tgtacaaaaa tagaaagttc aagcgtcctc agtaagcctt gttcagtgac tgtagccagt |
| 1381 | gaggcaagca agaagaaagt agatgttatt gatcttacaa tagaaagctc ttctgacgaa |
| 1441 | gaggaagacc ctcctgccaa aaggaaatgc atctttatgt cagaaacaca aagcagccca |
| 1501 | accaaagggg ttctcatgta tcagccatct tctgtaaggg tgcccagtgt gacttcggtt |
| 1561 | gatcctgctg ctattccgcc ttcattaaca gactactcag taccattcca ccatacgcca |
| 1621 | atatcaagca tgtcatcaga tttgccagga gaacaaagaa gaaatgatat taataatgaa |
| 1681 | ctgaagcttg gaacatcttc tgatactgtg caacagtga |

SEQ ID NO: 12 Human PIAS2 (isoform 1) amino acid sequence

| | |
|---|---|
| 1 | madfeelrnm vssfrvselq vllgfagrnk sgrkhdllmr alhllksgcs pavqikirel |
| 61 | yrrryprtle glsdlstiks svfsldggss pvepdlavag ihslpstsvt phspsspvgs |
| 121 | vllqdtkptf emqqpsppip pvhpdvqlkn lpfydvldvl ikptslvqss iqrfqekffi |
| 181 | faltpqqvre icisrdflpg grrdytvqvq lrlclaetsc pqednypnsl cikvngklfp |
| 241 | lpgyapppkn gieqkrpgrp lnitslvrls savpnqisis waseigknys msvylvrqlt |
| 301 | samllqrlkm kgirnpdhsr alikekltad pdseiattsl rvslmcplgk mrltipcrav |
| 361 | tcthlqcfda alylqmnekk ptwicpvcdk kaayeslild glfmeilndc sdvdeikfqe |
| 421 | dgswcpmrpk keamkvssqp ctkiesssvl skpcsvtvas easkkkvdvi dltiesssde |
| 481 | eedppakrkc ifmsetqssp tkgvlmyqps svrvpsvtsv dpaaippslt dysvpfhhtp |
| 541 | issmssdlpg eqrrndinne lklgtssdtv qq |

SEQ ID NO: 13 Human PIAS2 (transcript variant 2) cDNA sequence

| | |
|---|---|
| 1 | atggcggatt tcgaagagtt gaggaatatg gtttctagtt ttagggtttc tgaactacaa |
| 61 | gtattactag gctttgctgg acggaataaa agtggacgca agcatgacct cctgatgagg |
| 121 | gcgctgcatt tattgaagag cggctgcagc cctgcggttc agattaaaat ccgagaattg |
| 181 | tatagacgcc gatatccacg aactcttgaa ggactttctg atttatccac aatcaaatca |
| 241 | tcggttttca gtttggatgg tggctcatca cctgtagaac ctgacttggc cgtggctgga |
| 301 | atccactcgt tgccttccac ttcagttaca cctcactcac catcctctcc tgttggttct |
| 361 | gtgctgcttc aagatactaa gcccacattt gagatgcagc agccatctcc cccaattcct |
| 421 | cctgtccatc ctgatgtgca gttaaaaaat ctgccctttt atgatgtcct tgatgttctc |
| 481 | atcaagccca cgagtttagt tcaaagcagt attcagcgat ttcaagagaa gttttttatt |
| 541 | tttgctttga cacctcaaca agttagagag atatgcatat ccagggattt tttgccaggt |
| 601 | ggtaggagag attatacagt ccaagttcag ttgagacttt gcctggcaga gacaagttgc |
| 661 | cctcaagaag ataactatcc aaatagtcta tgtataaaag taaatgggaa gctatttcct |
| 721 | ttgcctggct atgcaccacc gcctaaaaat gggattgaac agaagcgccc tggacgcccc |
| 781 | ttgaatatta catctttagt taggttatct tcagctgtgc caaaccaaat ttccatttct |
| 841 | tgggcatcag aaattgggaa gaattactct atgtctgtat atcttgtacg gcagcttaca |
| 901 | tcagccatgt tattacagag attaaaaatg aaaggtatta gaaaccctga tcattccaga |
| 961 | gcactaatta aagaaaaact tactgcagat cctgatagtg aaattgctac aactagcctt |
| 1021 | cgggtatcct tgatgtgccc tttaggaaaa atgaggctga caatcccatg ccgtgcagtg |
| 1081 | acttgtacac atctgcagtg ttttgatgct gccctctatc tacaaatgaa tgagaaaaag |
| 1141 | cccacctgga tttgtcctgt gtgtgacaaa aaagctgcct atgaaagtct aatattagat |
| 1201 | gggcttttta tggaaattct caatgactgt tctgatgtag atgagatcaa attccaagaa |
| 1261 | gatggttctt ggtgtccaat gagaccgaag aaagaagcta tgaaagtatc cagccaaccg |
| 1321 | tgtacaaaaa tagaaagttc aagcgtcctc agtaagcctt gttcagtgac tgtagccagt |
| 1381 | gaggcaagca agaagaaagt agatgttatt gatcttacaa tagaaagctc ttctgacgaa |
| 1441 | gaggaagacc ctcctgccaa aaggaaatgc atctttatgt cagaaacaca aagcagccca |
| 1501 | accaaagggg ttctcatgta tcagccatct tctgtaaggg tgcccagtgt gacttcggtt |
| 1561 | gatcctgctg ctattccgcc ttcattaaca gactactcag taccattcca ccatacgcca |
| 1621 | atatcaagca tgtcatcaga tttgccaggt ttggattttc tttcccttat tccagttgat |
| 1681 | ccccagtact gtcctcctat gtttttggat agtctcacct cacccttaac agcaagcagt |
| 1741 | acgtctgtca ccaccaccag ctcccatgaa agcagtactc atgttagttc atccagcagc |
| 1801 | aggagtgaga caggggtcat aaccagcagt ggaagtaaca ttcctgacat catctcattg |
| 1861 | gactaa |

SEQ ID NO: 14 Human PIAS2 (isoform 2) amino acid sequence

| | |
|---|---|
| 1 | madfeelrnm vssfrvselq vllgfagrnk sgrkhdllmr alhllksgcs pavqikirel |
| 61 | yrrryprtle glsdlstiks svfsldggss pvepdlavag ihslpstsvt phspsspvgs |
| 121 | vllqdtkptf emqqpsppip pvhpdvqlkn lpfydvldvl ikptslvqss iqrfqekffi |
| 181 | faltpqqvre icisrdflpg grrdytvqvq lrlclaetsc pqednypnsl cikvngklfp |
| 241 | lpgyapppkn gieqkrpgrp lnitslvrls savpnqisis waseigknys msvylvrqlt |
| 301 | samllqrlkm kgirnpdhsr alikekltad pdseiattsl rvslmcplgk mrltipcrav |
| 361 | tcthlqcfda alylqmnekk ptwicpvcdk kaayeslild glfmeilndc sdvdeikfqe |
| 421 | dgswcpmrpk keamkvssqp ctkiesssvl skpcsvtvas easkkkvdvi dltiesssde |
| 481 | eedppakrkc ifmsetqssp tkgvlmyqps svrvpsvtsv dpaaippslt dysvpfhhtp |
| 541 | issmssdlpg idflslipvd pqycppmfld sltspltass tsvtttsshe ssthvsssss |
| 601 | rsetgvitss gsnpdiisl d |

SEQ ID NO: 15 Human PIAS3 cDNA sequence

| | |
|---|---|
| 1 | atggcggagc tgggcgaatt aaagcacatg gtgatgagtt tccgggtgtc tgagctccag |
| 61 | gtgcttcttg gctttgctgg ccggaacaag agtggacgga agcacgagct cctggccaag |
| 121 | gctctgcacc tcctgaagtc cagctgtgcc cctagtgtcc agatgaagat caaagagctt |
| 181 | taccgacgac gctttccccg gaagaccctg gggccctctg atctctccct tctctctttg |
| 241 | ccccctggca cctctcctgt aggctcccct ggtcctctag ctcccattcc cccaacgctg |
| 301 | ttggcccctg gcaccctgct gggccccaag cgtgaggtgg acatgcaccc ccctctgccc |
| 361 | cagcctgtgc accctgatgt caccatgaaa ccattgccct tctatgaagt ctatggggag |
| 421 | ctcatccggc ccaccaccct tgcatccact tctagccagc ggtttgagga agcgcacttt |
| 481 | acctttgccc tcacacccca gcaagtgcag cagattctta catccagaga ggttctgcca |
| 541 | ggagccaaat gtgattatac catacaggtg cagctaaggt tctgtctctg tgagaccagc |
| 601 | tgcccccagg aagattattt tccccccaac ctctttgtca aggtcaatgg gaaactgtgc |
| 661 | cccctgccgg gttaccttcc cccaaccaag aatggggccg agcccaagag gcccagccgc |
| 721 | cccatcaaca tcacacccct ggctcgactc tcagccactg ttcccaacac cattgtggtc |
| 781 | aattggtcat ctgagttcgg acggaattac tccttgtctg tgtacctggt gaggcagttg |
| 841 | actgcaggaa cccttctaca aaaactcaga gcaaagggta tccggaaccc agaccactcg |
| 901 | cgggcactga tcaaggagaa attgactgct gaccctgaca gtgaggtggc cactacaagt |
| 961 | ctccgggtgt cactcatgtg cccgctaggg aagatgcgcc tgactgtccc ttgtcgtgcc |
| 1021 | ctcacctgcg cccacctgca gagcttcgat gctgcccttt atctacagat gaatgagaag |
| 1081 | aagcctacat ggacatgtcc tgtgtgtgac aagaaggctc cctatgaatc tcttatcatt |
| 1141 | gatggtttat ttatggagat tcttagttcc tgttcagatt gtgatgagat ccaattcatg |
| 1201 | gaagatggat cctggtgccc aatgaaaccc aagaaggagg catctgaggt ttgccccccg |
| 1261 | ccagggtatg ggctggatgg cctccagtac agcccagtcc aggggggaga tccatcagag |
| 1321 | aataagaaga aggtcgaagt tattgacttg acaatagaaa gctcatcaga tgaggaggat |
| 1381 | ctgcccccta ccaagaagca ctgttctgtc acctcagctg ccatcccggc cctacctgga |
| 1441 | agcaaaggag tcctgacatc tggccaccag ccatcctcgg tgctaaggag ccctgctatg |
| 1501 | ggcacgttgg gtggggattt cctgtccagt ctcccactac atgagtaccc acctgccttc |
| 1561 | ccactgggag ccgacatcca aggtttagat ttattttcat ttcttcagac agagagtcag |
| 1621 | cactatggcc cctctgtcat cacctcacta gatgaacagg atgcccttgg ccacttcttc |
| 1681 | cagtaccgag ggaccccttc tcactttctg ggcccactgg cccccacgct ggggagctcc |
| 1741 | cactgcagcg ccactccggc gccccctcct ggccgtgtca gcagcattgt ggcccctggg |
| 1801 | ggggccttga gggaggggca tggaggaccc ctgccctcag gtccctcttt gactggctgt |
| 1861 | cggtcagaca tcatttccct ggactga |

SEQ ID NO: 16 Human PIAS3 amino acid sequence

| | |
|---|---|
| 1 | maelgelkhm vmsfrvselq vllgfagrnk sgrkhellak alhllkssca psvqmkikel |
| 61 | yrrrfprktl qpsdlsllsl ppgtspvgsp gplapipptl lapgtllgpk revdmhpplp |
| 121 | qpvhpdvtmk plpfyevyge lirpttlast ssqrfeeahf tfaltpqqvq qiltsrevlp |
| 181 | gakcdytiqv qlrfclcets cpqedyfppn lfvkvngklc plpgylpptk ngaepkrpsr |
| 241 | pinitplarl satvpntivv nwssefgrny slsvylvrql tagtllqklr akgirnpdhs |
| 301 | ralikeklta dpdsevatts lrvslmcplg kmrltvpcra ltcahlqsfd aalylqmnek |
| 361 | kptwtcpvcd kkapyeslii dglfmeilss csdcdeiqfm edgswcpmkp kkeasevcpp |
| 421 | pgygldglqy spvqggdpse nkkkvevidl tiesssdeed lpptkkhcsv tsaaipalpg |
| 481 | skgvltsghq pssvlrspam gtlggdflss lplheyppaf plgadiqgld lfsflqtesq |
| 541 | hygpsvitsl deqdalghff qyrgtpshfl gplaptlgss hcsatpappp grvssivapg |
| 601 | galreghggp lpsgpsltgc rsdiisld |

SEQ ID NO: 17 Human PIAS4 cDNA sequence

| | |
|---|---|
| 1 | atggcggcgg agctggtgga ggccaaaaac atggtgatga gttttcgagt ctccgacctt |
| 61 | cagatgctcc tgggtttcgt gggccggagt aagagtggac tgaagcacga gctcgtcacc |
| 121 | agggccctcc agctggtgca gtttgactgt agccctgagc tgttcaagaa gatcaaggag |
| 181 | ctgtacgaga cccgctacgc caagaagaac tcggagcctg ccccacagcc gcaccggccc |
| 241 | ctggaccccc tgaccatgca ctccacctac gaccgggccg gcgctgtgcc caggactccg |
| 301 | ctggcaggcc ccaatattga ctaccccgtg ctctacggaa agtacttaaa cggactggga |
| 361 | cggttgcccg ccaagaccct caagccagaa gtccgcctgg tgaagctgcc gttctttaat |
| 421 | atgctggatg agctgctgaa gcccaccgaa ttagtcccac agaacaacga gaagcttcag |
| 481 | gagagcccgt gcatcttcgc attgacgcca agacaggtgg agttgatccg gaactccagg |
| 541 | gaactgcagc ccggagttaa agccgtgcag gtcgtcctga gaatctgtta ctcagacacc |
| 601 | agctgccctc aggaggacca gtacccgccc aacatcgctg tgaaggtcaa ccacagctac |
| 661 | tgctccgtcc cgggctacta cccctccaat aagcccgggg tggagcccaa gaggccgtgc |
| 721 | cgccccatca acctcactca cctcatgtac ctgtcctcgg ccaccaaccg catcactgtc |
| 781 | acctggggga actacggcaa gagctactcg gtggccctgt acctggtgcg gcagctgacc |
| 841 | tcatcggagc tgctgcagag gctgaagacc attggggtaa agcacccgga gctgtgcaag |
| 901 | gcactggtca aggagaagct gcgccttgat cctgacagcg agatcgccac caccggtgtg |
| 961 | cgggtgtccc tcatctgtcc gctggtgaag atgcggctct ccgtgccctg ccgggcagag |
| 1021 | acctgcgccc acctgcagtg cttcgacgcc gtcttctacc tgcagatgaa cgagaagaag |
| 1081 | cccacctgga tgtgccccgt gtgcgacaag ccagccccct acgaccagct catcatcgac |
| 1141 | gggctcctct cgaagatcct gagcgagtgt gaggacgccg acgagatcga gtacctggtg |
| 1201 | gacggctcgt ggtgcccgat ccgcgccgaa aaggagcgca gctgcagccc gcagggcgcc |
| 1261 | atcctcgtgc tgggcccctc ggacgccaat gggctcctgc ccgcccccag cgtcaacggg |
| 1321 | agcggtgccc tgggcagcac gggtggcggc ggcccggtgg gcagcatgga gaatgggaag |
| 1381 | ccgggcgccg atgtggtgga cctcacgctg gacagctcat cgtcctcgga ggatgaggag |
| 1441 | gaggaggaag aggaggagga agacgaggac gaagaggggc cccggcccaa gcgccgctgc |
| 1501 | cccttccaga agggcctggt gccggcctgc tga |

SEQ ID NO: 18 Human PIAS4 amino acid sequence

| | |
|---|---|
| 1 | maaelveakn mvmsfrvsdl qmllgfvgrs ksglkhelvt ralqlvqfdc spelfkkike |
| 61 | lyetryakkn sepapqphrp ldpltmhsty dragavprtp lagpnidypv lygkylnglg |
| 121 | rlpaktlkpe vrlvklpffn mldellkpte lvpqnneklq espcifaltp rqvelirnsr |
| 181 | elqpgvkavq vvlricysdt scpqedqypp niavkvnhsy csvpgyypsn kpgvepkrpc |
| 241 | rpinlthlmy lssatnritv twgnygksys valylvrqlt ssellqrlkt igvkhpelck |
| 301 | alvkeklrld pdseiattgv rvslicplvk mrlsvpcrae tcahlqcfda vfylqmnekk |
| 361 | ptwmcpvcdk papydqliid gllskilsec edadeieylv dgswcpirae kerscspqga |
| 421 | ilvlgpsdan gllpapsvng sgalgstggg gpvgsmengk pgadvvdltl dsssssedee |
| 481 | eeeeeeeded eegprpkrrc pfqkglvpac |

SEQ ID NO: 19 Human SOC1 cDNA sequence

| | |
|---|---|
| 1 | atggtagcac acaaccaggt ggcagccgac aatgcagtct ccacagcagc agagccccga |
| 61 | cggcggccag aaccttcctc ctcttcctcc tcctcgcccg cggcccccgc gcgcccgcgg |
| 121 | ccgtgccccg cggtcccggc cccggccccc ggcgacacgc acttccgcac attccgttcg |
| 181 | cacgccgatt accggcgcat cacgcgcgcc agcgcgctcc tggacgcctg cggattctac |
| 241 | tgggggcccc tgagcgtgca cggggcgcac gagcggctgc gcgccgagcc cgtgggcacc |
| 301 | ttcctggtgc gcgacagccg ccagcggaac tgctttttcg cccttagcgt gaagatggcc |
| 361 | tcgggaccca cgagcatccg cgtgcacttt caggccggcc gctttcacct ggatggcagc |
| 421 | cgcgagagct tcgactgcct cttcgagctg ctggagcact acgtggcggc gccgcgccgc |
| 481 | atgctggggg ccccgctgcg ccagcgccgc gtgcggccgc tgcaggagct gtgccgccag |
| 541 | cgcatcgtgg ccaccgtggg ccgcgagaac ctggctcgca tccccctcaa ccccgtcctc |
| 601 | cgcgactacc tgagctcctt ccccttccag atttga |

SEQ ID NO: 20 Human SOCS1 amino acid sequence

| | |
|---|---|
| 1 | mvahnqvaad navstaaepr rrpepsssss sspaaparpr pcpavpapap gdthfrtfrs |
| 61 | hadyrritra salldacgfy wgplsvhgah erlraepvgt flvrdsrqrn cffalsvkma |
| 121 | sgptsirvhf qagrfhldgs resfdclfel lehyvaaprr mlgaplrqrr vrplqelcrq |
| 181 | rivatvgren lariplnpvl rdylssfpfq i |

SEQ ID NO: 21 Human SOCS3 cDNA sequence

| | |
|---|---|
| 1 | atggtcaccc acagcaagtt tcccgccgcc gggatgagcc gccccctgga caccagcctg |
| 61 | cgcctcaaga ccttcagctc caagagcgag taccagctgg tggtgaacgc agtgcgcaag |
| 121 | ctgcaggaga gcggcttcta ctggagcgca gtgaccggcg gcgaggcgaa cctgctgctc |
| 181 | agtgccgagc ccgccggcac ctttctgatc cgcgacagct cggaccagcg ccacttcttc |
| 241 | acgctcagcg tcaagaccca gtctgggacc aagaacctgc gcatccagtg tgaggggggc |
| 301 | agcttctctc tgcagagcga tccccggagc acgcagcccg tgccccgctt cgactgcgtg |
| 361 | ctcaagctgg tgcaccacta catgccgccc cctggagccc cctccttccc ctcgccacct |
| 421 | actgaaccct cctccgaggt gcccgagcag ccgtctgccc agccactccc tgggagtccc |
| 481 | cccagaagag cctattacat ctactccggg ggcgagaaga tccccctggt gttgagccgg |
| 541 | cccctctcct ccaacgtggc cactcttcag catctctgtc ggaagaccgt caacggccac |
| 601 | ctggactcct atgagaaagt cacccagctg ccggggccca ttcgggagtt cctggaccag |
| 661 | tacgatgccc cgctttaa |

SEQ ID NO: 22 Human SOC3 amino acid sequence

| | |
|---|---|
| 1 | mvthskfpaa gmsrpldtsl rlktfsskse yqlvvnavrk lqesgfywsa vtggeanlll |
| 61 | saepagtfli rdssdqrhff tlsvktqsgt knlriqcegg sfslqsdprs tqpvprfdcv |
| 121 | lklvhhympp pgapsfpspp tepssevpeq psaqplpgsp prrayyiysg gekiplvlsr |
| 181 | plssnvatlq hlcrktvngh ldsyekvtql pgpirefldq ydapl |

SEQ ID NO: 23 Human SHP-1 (transcript variant 1) cDNA sequence

| | |
|---|---|
| 1 | atggtgaggt ggtttcaccg agacctcagt gggctggatg cagagaccct gctcaagggc |
| 61 | cgaggtgtcc acggtagctt cctggctcgg cccagtcgca agaaccaggg tgacttctcg |
| 121 | ctctccgtca gggtggggga tcaggtgacc catattcgga tccagaactc aggggatttc |
| 181 | tatgacctgt atggagggga gaagtttgcg actctgacag agctggtgga gtactacact |
| 241 | cagcagcagg gtgtcctgca ggaccgcgac ggcaccatca tccacctcaa gtacccgctg |
| 301 | aactgctccg atcccactag tgagaggtgg taccatggcc acatgtctgg cgggcaggca |
| 361 | gagacgctgc tgcaggccaa gggcgagccc tggacgtttc ttgtgcgtga gagcctcagc |
| 421 | cagcctggag acttcgtgct ttctgtgctc agtgaccagc ccaaggctgg cccaggctcc |
| 481 | ccgctcaggg tcacccacat caaggtcatg tgcgagggtg gacgctacac agtgggtggt |
| 541 | ttggagacct tcgacagcct cacggacctg gtggagcatt tcaagaagac ggggattgag |
| 601 | gaggcctcag gcgcctttgt ctacctgcgg cagccgtact atgccacgag ggtgaatgcg |
| 661 | gctgacattg agaaccgagt gttggaactg aacaagaagc aggagtccga ggatacagcc |
| 721 | aaggctggct tctgggagga gtttgagagt ttgcagaagc aggaggtgaa gaacttgcac |
| 781 | cagcgtctgg aagggcagcg gccagagaac aagggcaaga accgctacaa gaacattctc |
| 841 | ccctttgacc acagccgagt gatcctgcag ggacgggaca gtaacatccc cgggtccgac |
| 901 | tacatcaatg ccaactacat caagaaccag ctgctaggcc ctgatgagaa cgctaagacc |
| 961 | tacatcgcca gccagggctg tctggaggcc acggtcaatg acttctggca gatggcgtgg |
| 1021 | caggagaaca gccgtgtcat cgtcatgacc acccgagagg tggagaaagg ccggaacaaa |
| 1081 | tgcgtcccat actggcccga ggtgggcatg cagcgtgctt atgggcccta ctctgtgacc |
| 1141 | aactgcgggg agcatgacac aaccgaatac aaactccgta ccttacaggt ctccccgctg |
| 1201 | gacaatggag acctgattcg ggagatctgg cattaccagt acctgagctg gcccgaccat |
| 1261 | ggggtcccca gtgagcctgg gggtgtcctc agcttcctgg accagatcaa ccagcggcag |
| 1321 | gaaagtctgc ctcacgcagg gcccatcatc gtgcactgca gcgccggcat cggccgcaca |
| 1381 | ggcaccatca ttgtcatcga catgctcatg gagaacatct ccaccaaggg cctggactgt |
| 1441 | gacattgaca tccagaagac catccagatg gtgcgggcgc agcgctcggg catggtgcag |
| 1501 | acggaggcgc agtacaagtt catctacgtg gccatcgccc agttcattga aaccactaag |
| 1561 | aagaagctgg aggtcctgca gtcgcagaag ggccaggagt cggagtacgg gaacatcacc |
| 1621 | tatcccccag ccatgaagaa tgcccatgcc aaggcctccc gcacctcgtc caaacacaag |
| 1681 | gaggatgtgt atgagaacct gcacactaag aacaagaggg aggagaaagt gaagaagcag |
| 1741 | cggtcagcag acaaggagaa gagcaagggt tccctcaaga ggaagtga |

SEQ ID NO: 24 Human SHP-1 (isoform 1) amino acid sequence

| | |
|---|---|
| 1 | mvrwfhrdls gldaetllkg rgvhgsflar psrknqgdfs lsvrvgdqvt hiriqnsgdf |
| 61 | ydlyggekfa tltelveyyt qqqgvlqdrd gtiihlkypl ncsdptserw yhghmsggqa |
| 121 | etllqakgep wtflvresls qpgdfvlsvl sdqpkagpgs plrvthikvm ceggrytvgg |
| 181 | letfdsltdl vehfkktgie easgafvylr qpyyatrvna adienrvlel nkkqesedta |
| 241 | kagfweefes lqkqevknlh qrlegqrpen kgknryknil pfdhsrvilq grdsnipgsd |
| 301 | yinanyiknq llgpdenakt yiasqgclea tvndfwqmaw qensrvivmt trevekgrnk |
| 361 | cvpywpevgm qraygpysvt ncgehdttey klrtlqvspl dngdlireiw hyqylswpdh |
| 421 | gvpsepggvl sfldqinqrq eslphagpii vhcsagigrt gtiividmlm enistkgldc |
| 481 | didiqktiqm vraqrsgmvq teaqykfiyv aiaqfiettk kklevlqsqk gqeseygnit |
| 541 | yppamknaha kasrtsskhk edvyenlhtk nkreekvkkq rsadkekskg slkrk |

SEQ ID NO: 25 Human SHP-1 (transcript variant 2) cDNA sequence

| | |
|---|---|
| 1 | atgctgtccc gtgggtggtt tcaccgagac ctcagtgggc tggatgcaga gaccctgctc |
| 61 | aagggccgag gtgtccacgg tagcttcctg gctcggccca gtcgcaagaa ccagggtgac |
| 121 | ttctcgctct ccgtcagggt gggggatcag gtgacccata ttcggatcca gaactcaggg |
| 181 | gatttctatg acctgtatgg aggggagaag tttgcgactc tgacagagct ggtggagtac |
| 241 | tacactcagc agcagggtgt cctgcaggac cgcgacggca ccatcatcca cctcaagtac |
| 301 | ccgctgaact gctccgatcc cactagtgag aggtggtacc atggccacat gtctggcggg |
| 361 | caggcagaga cgctgctgca ggccaagggc gagccctgga cgtttcttgt gcgtgagagc |
| 421 | ctcagccagc ctggagactt cgtgctttct gtgctcagtg accagcccaa ggctggccca |
| 481 | ggctccccgc tcagggtcac ccacatcaag gtcatgtgcg agggtggacg ctacacagtg |
| 541 | ggtggtttgg agaccttcga cagcctcacg gacctggtgg agcatttcaa gaagacgggg |
| 601 | attgaggagg cctcaggcgc ctttgtctac ctgcggcagc cgtactatgc cacgagggtg |
| 661 | aatgcggctg acattgagaa ccgagtgttg gaactgaaca agaagcagga gtccgaggat |
| 721 | acagccaagg ctggcttctg ggaggagttt gagagtttgc agaagcagga ggtgaagaac |
| 781 | ttgcaccagc gtctggaagg gcagcggcca gagaacaagg gcaagaaccg ctacaagaac |
| 841 | attctcccct ttgaccacag ccgagtgatc ctgcagggac gggacagtaa catccccggg |
| 901 | tccgactaca tcaatgccaa ctacatcaag aaccagctgc taggccctga tgagaacgct |
| 961 | aagacctaca tcgccagcca gggctgtctg gaggccacgg tcaatgactt ctggcagatg |
| 1021 | gcgtggcagg agaacagccg tgtcatcgtc atgaccaccc gagaggtgga gaaaggccgg |
| 1081 | aacaaatgcg tcccatactg gcccgaggtg ggcatgcagc gtgcttatgg gccctactct |
| 1141 | gtgaccaact gcggggagca tgacacaacc gaatacaaac tccgtacctt acaggtctcc |
| 1201 | ccgctggaca atggagacct gattcgggag atctggcatt accagtacct gagctggccc |
| 1261 | gaccatgggg tccccagtga gcctgggggt gtcctcagct tcctggacca gatcaaccag |
| 1321 | cggcaggaaa gtctgcctca cgcagggccc atcatcgtgc actgcagcgc cggcatcggc |
| 1381 | cgcacaggca ccatcattgt catcgacatg ctcatggaga acatctccac caagggcctg |
| 1441 | gactgtgaca ttgacatcca gaagaccatc cagatggtgc gggcgcagcg ctcgggcatg |
| 1501 | gtgcagacgg aggcgcagta caagttcatc tacgtggcca tcgcccagtt cattgaaacc |
| 1561 | actaagaaga agctggaggt cctgcagtcg cagaagggcc aggagtcgga gtacgggaac |
| 1621 | atcacctatc ccccagccat gaagaatgcc catgccaagg cctcccgcac ctcgtccaaa |
| 1681 | cacaaggagg atgtgtatga gaacctgcac actaagaaca agagggagga gaaagtgaag |
| 1741 | aagcagcggt cagcagacaa ggagaagagc aagggttccc tcaagaggaa gtga |

SEQ ID NO: 26 Human SHP-1 (isoform 2) amino acid sequence

| | |
|---|---|
| 1 | mlsrgwfhrd lsgldaetll kgrgvhgsfl arpsrknqgd fslsvrvgdq vthiriqnsg |
| 61 | dfydlyggek fatltelvey ytqqqgvlqd rdgtiihlky plncsdptse rwyhghmsgg |
| 121 | qaetllqakg epwtflvres lsqpgdfvls vlsdqpkagp gsplrvthik vmceggrytv |
| 181 | ggletfdslt dlvehfkktg ieeasgafvy lrqpyyatrv naadienrvl elnkkqesed |
| 241 | takagfweef eslqkqevkn lhqrlegqrp enkgknrykn ilpfdhsrvi lqgrdsnipg |
| 301 | sdyinanyik nqllgpdena ktyiasqgcl eatvndfwqm awqensrviv mttrevekgr |
| 361 | nkcvpywpev gmqraygpys vtncgehdtt eyklrtlqvs pldngdlire iwhyqylswp |
| 421 | dhgvpsepgg vlsfldqinq rqeslphagp iivhcsagig rtgtiividm lmenistkgl |
| 481 | dcdidiqkti qmvraqrsgm vqteaqykfi yvaiaqfiet tkkklevlqs qkgqeseygn |
| 541 | ityppamkna hakasrtssk hkedvyenlh tknkreekvk kqrsadkeks kgslkrk |

SEQ ID NO: 27 Human SHP-1 (transcript variant 3) cDNA sequence

| | |
|---|---|
| 1 | atggtgaggt ggtttcaccg agacctcagt gggctggatg cagagaccct gctcaagggc |
| 61 | cgaggtgtcc acggtagctt cctggctcgg cccagtcgca agaaccaggg tgacttctcg |
| 121 | ctctccgtca gggtggggga tcaggtgacc catattcgga tccagaactc aggggatttc |
| 181 | tatgacctgt atggagggga gaagtttgcg actctgacag agctggtgga gtactacact |
| 241 | cagcagcagg gtgtcctgca ggaccgcgac ggcaccatca tccacctcaa gtacccgctg |
| 301 | aactgctccg atcccactag tgagaggtgg taccatggcc acatgtctgg cgggcaggca |
| 361 | gagacgctgc tgcaggccaa gggcgagccc tggacgtttc ttgtgcgtga gagcctcagc |
| 421 | cagcctggag acttcgtgct ttctgtgctc agtgaccagc ccaaggctgg cccaggctcc |
| 481 | ccgctcaggg tcacccacat caaggtcatg tgcgagggtg gacgctacac agtgggtggt |
| 541 | ttggagacct tcgacagcct cacggacctg gtggagcatt tcaagaagac ggggattgag |
| 601 | gaggcctcag gcgcctttgt ctacctgcgg cagccgtact atgccacgag ggtgaatgcg |
| 661 | gctgacattg agaaccgagt gttggaactg aacaagaagc aggagtccga ggatacagcc |
| 721 | aaggctggct tctgggagga gtttgagagt ttgcagaagc aggaggtgaa gaacttgcac |
| 781 | cagcgtctgg aagggcagcg gccagagaac aagggcaaga accgctacaa gaacattctc |
| 841 | ccctttgacc acagccgagt gatcctgcag ggacgggaca gtaacatccc cgggtccgac |
| 901 | tacatcaatg ccaactacat caagaaccag ctgctaggcc ctgatgagaa cgctaagacc |
| 961 | tacatcgcca gccagggctg tctggaggcc acggtcaatg acttctggca gatggcgtgg |
| 1021 | caggagaaca gccgtgtcat cgtcatgacc acccgagagg tggagaaagg ccggaacaaa |
| 1081 | tgcgtcccat actggcccga ggtgggcatg cagcgtgctt atgggcccta ctctgtgacc |
| 1141 | aactgcgggg agcatgacac aaccgaatac aaactccgta ccttacaggt ctccccgctg |
| 1201 | gacaatggag acctgattcg ggagatctgg cattaccagt acctgagctg gcccgaccat |
| 1261 | ggggtcccca gtgagcctgg gggtgtcctc agcttcctgg accagatcaa ccagcggcag |
| 1321 | gaaagtctgc ctcacgcagg gcccatcatc gtgcactgca gcgccggcat cggccgcaca |
| 1381 | ggcaccatca ttgtcatcga catgctcatg gagaacatct ccaccaaggg cctggactgt |
| 1441 | gacattgaca tccagaagac catccagatg gtgcgggcgc agcgctcggg catggtgcag |
| 1501 | acggaggcgc agtacaagtt catctacgtg gccatcgccc agttcattga aaccactaag |
| 1561 | aagaagctgg aggtcctgca gtcgcagaag ggccaggagt cggagtacgg gaacatcacc |
| 1621 | gagtctagtg cagggaccgt ggctgcgtca cctgtgagac ggggtggcca gaggggactg |
| 1741 | ccagtgccgg gtccccctgt gctgtctcct gacctgcacc aactgcctgt acttgccccc |
| 1801 | ctgcacccgg ctgcagacac aaggaggatg tgtatgagaa cctgcacact aagaacaaga |
| 1861 | gggaggagaa agtga |

SEQ ID NO: 28 Human SHP-1 (isoform 3) amino acid sequence

| | |
|---|---|
| 1 | mvrwfhrdls gldaetllkg rgvhgsflar psrknqgdfs lsvrvgdqvt hiriqnsgdf |
| 61 | ydlyggekfa tltelveyyt qqqgvlqdrd gtiihlkypl ncsdptserw yhghmsggqa |
| 121 | etllqakgep wtflvresls qpgdfvlsvl sdqpkagpgs plrvthikvm ceggrytvgg |
| 181 | letfdsltdl vehfkktgie easgafvylr qpyyatrvna adienrvlel nkkqesedta |
| 241 | kagfweefes lqkqevhnlh qrlegqrpen kgknryknil pfdhsrvilq grdsnipgsd |
| 301 | yinanyiknq llgpdenakt yiasqgclea tvndfwqmaw qensrvivmt trevekgrnk |
| 361 | cvpywpevgm qraygpysvt ncgehdttey klrtlqvspl dngdlireiw hyqylswpdh |
| 421 | gvpsepggvl sfldqinqrq eslphagpii vhcsagigrt gtiividmlm enistlgldc |
| 481 | didiqktiqm vraqrsgmvq teaqykfiyv aiaqfiettk kklevlqsqk gqeseygnit |
| 541 | yppamknaha kasrtssdsl essagtvaas pvrrggqrgl pvpgppvlsp dlhqlpvlap |
| 601 | lhpaadtrrm cmrtctlrtr grrk |

SEQ ID NO: 29 Human SHP-2 (transcript variant 1) cDNA sequence

| | |
|---|---|
| 1 | atgacatcgc ggagatggtt tcacccaaat atcactggtg tggaggcaga aaacctactg |
| 61 | ttgacaagag gagttgatgg cagttttttg gcaaggccta gtaaaagtaa ccctggagac |
| 121 | ttcacacttt ccgttagaag aaatggagct gtcacccaca tcaagattca gaacactggt |
| 181 | gattactatg acctgtatgg aggggagaaa tttgccactt tggctgagtt ggtccagtat |
| 241 | tacatggaac atcacgggca attaaaagag aagaatggag atgtcattga gcttaaatat |
| 301 | cctctgaact gtgcagatcc tacctctgaa aggtggtttc atggacatct ctctgggaaa |
| 361 | gaagcagaga aattattaac tgaaaaagga aaacatggta gttttcttgt acgagagagc |
| 421 | cagagccacc ctggagattt tgttctttct gtgcgcactg gtgatgacaa aggggagagc |
| 481 | aatgacggca agtctaaagt gacccatgtt atgattcgct gtcaggaact gaaatacgac |
| 541 | gttggtggag gagaacggtt tgattctttg acagatcttg tggaacatta taagaagaat |
| 601 | cctatggtgg aaacattggg tacagtacta caactcaagc agccccttaa cacgactcgt |
| 661 | ataaatgctg ctgaaataga aagcagagtt cgagaactaa gcaaattagc tgagaccaca |
| 721 | gataaagtca aacaaggctt ttgggaagaa tttgagacac tacaacaaca ggagtgcaaa |
| 781 | cttctctaca gccgaaaaga gggtcaaagg caagaaaaca aaaacaaaaa tagatataaa |
| 841 | aacatcctgc cctttgatca taccagggtt gtcctacacg atggtgatcc caatgagcct |
| 901 | gtttcagatt acatcaatgc aaatatcatc atgcctgaat ttgaaaccaa gtgcaacaat |
| 961 | tcaaagccca aaaagagtta cattgccaca caaggctgcc tgcaaaacac ggtgaatgac |
| 1021 | ttttggcgga tggtgttcca agaaaactcc cgagtgattg tcatgacaac gaaagaagtg |
| 1081 | gagagaggaa agagtaaatg tgtcaaatac tggcctgatg agtatgctct aaaagaatat |
| 1141 | ggcgtcatgc gtgttaggaa cgtcaaagaa agcgccgctc atgactatac gctaagagaa |
| 1201 | cttaaacttt caaaggttgg acaagggaat acggagagaa cggtctggca ataccacttt |
| 1261 | cggacctggc cggaccacgg cgtgcccagc gaccctgggg gcgtgctgga cttcctggag |
| 1321 | gaggtgcacc ataagcagga gagcatcatg gatgcagggc cggtcgtggt gcactgcagt |
| 1381 | gctggaattg gccggacagg gacgttcatt gtgattgata ttcttattga catcatcaga |
| 1441 | gagaaaggtg ttgactgcga tattgacgtt cccaaaacca tccagatggt gcggtctcag |
| 1501 | aggtcaggga tggtccagac agaagcacag taccgattta tctatatggc ggtccagcat |
| 1561 | tatattgaaa cactacagcg caggattgaa gaagagcaga aaagcaagag gaaagggcac |
| 1621 | gaatatacaa atattaagta ttctctagcg gaccagacga gtggagatca gagccctctc |
| 1681 | ccgccttgta ctccaacgcc accctgtgca gaaatgagag aagacagtgc tagagtctat |
| 1741 | gaaaacgtgg gcctgatgca acagcagaaa agtttcagat ga |

SEQ ID NO: 30 Human SHP-2 (isoform 1) amino acid sequence

| | |
|---|---|
| 1 | mtsrrwfhpn itgveaenll ltrgvdgsfl arpsksnpgd ftlsvrrnga vthikiqntg |
| 61 | dyydlyggek fatlaelvqy ymehhgqlke kngdvielky plncadptse rwfhghlsgk |
| 121 | eaeklltekg khgsflvres qshpgdfvls vrtgddkges ndgkskvthv mircqelkyd |
| 181 | vgggerfdsl tdlvehykkn pmvetlgtvl qlkqplnttr inaaeiesrv relsklaett |
| 241 | dkvkqgfwee fetlqqqeck llysrkegqr qenknknryk nilpfdhtrv vlhdgdpnep |
| 301 | vsdyinanii mpefetkcnn skpkksyiat qqclqntvnd fwrmvfeqns rvivmttkev |
| 361 | ergkskcvky wpedyalkey gvmrvrnvke saahdytlre lklskvgqgn tertvwqyhf |
| 421 | rtwpdhgvps dpggvldfle erhhkqesim dagpvvvhcs agigrtgtfi vidilidiir |
| 481 | ekgvdcdidv pktiqmvrsq rsgmvqteaq yrfiymavqh yietlqrrie eeqkskrkgh |
| 541 | eytnikysla dqtsgdqspl ppctptppca emredsarvy envglmqqqk sfr |

SEQ ID NO: 31 Human SHP-2 (transcript variant 1) cDNA sequence

| | |
|---|---|
| 1 | atgacatcgc ggagatggtt tcacccaaat atcactggtg tggaggcaga aaacctactg |
| 61 | ttgacaagag gagttgatgg cagttttttg gcaaggccta gtaaaagtaa ccctggagac |
| 121 | ttcacacttt ccgttagaag aaatggagct gtcacccaca tcaagattca gaacactggt |
| 181 | gattactatg acctgtatgg aggggagaaa tttgccactt tggctgagtt ggtccagtat |
| 241 | tacatggaac atcacgggca attaaaagag aagaatggag atgtcattga gcttaaatat |
| 301 | cctctgaact gtgcagatcc tacctctgaa aggtggtttc atggacatct ctctgggaaa |
| 361 | gaagcagaga aattattaac tgaaaaagga aaacatggta gttttcttgt acgagagagc |
| 421 | cagagccacc ctggagattt tgttctttct gtgcgcactg gtgatgacaa aggggagagc |
| 481 | aatgacggca agtctaaagt gacccatgtt atgattcgct gtcaggaact gaaatacgac |
| 541 | gttggtggag gagaacggtt tgattctttg acagatcttg tggaacatta taagaagaat |
| 601 | cctatggtgg aaacattggg tacagtacta caactcaagc agccccttaa cacgactcgt |
| 661 | ataaatgctg ctgaaataga aagcagagtt cgagaactaa gcaaattagc tgagaccaca |
| 721 | gataaagtca aacaaggctt ttgggaagaa tttgagacac tacaacaaca ggagtgcaaa |
| 781 | cttctctaca gccgaaaaga gggtcaaagg caagaaaaca aaaacaaaaa tagatataaa |
| 841 | aacatcctgc cctttgatca taccagggtt gtcctacacg atggtgatcc caatgagcct |
| 901 | gtttcagatt acatcaatgc aaatatcatc atgcctgaat ttgaaaccaa gtgcaacaat |
| 961 | tcaaagccca aaaagagtta cattgccaca caaggctgcc tgcaaaacac ggtgaatgac |
| 1021 | ttttggcgga tggtgttcca agaaaactcc cgagtgattg tcatgacaac gaaagaagtg |
| 1081 | gagagaggaa agagtaaatg tgtcaaatac tggcctgatg agtatgctct aaaagaatat |
| 1141 | ggcgtcatgc gtgttaggaa cgtcaaagaa agcgccgctc atgactatac gctaagagaa |
| 1201 | cttaaacttt caaaggttgg acaagggaat acggagagaa cggtctggca ataccacttt |
| 1261 | cggacctggc cggaccacgg cgtgcccagc gaccctgggg gcgtgctgga cttcctggag |
| 1321 | gaggtgcacc ataagcagga gagcatcatg gatgcagggc cggtcgtggt gcactgcagg |
| 1381 | tga |

SEQ ID NO: 32 Human SHP-2 (isoform 2) amino acid sequence

| | |
|---|---|
| 1 | mtsrrwfhpn itgveaenll ltrgvdgsfl arpsksnpgd ftlsvrrnga vthikiqntg |
| 61 | dyydlyggek fatlaelvqy ymehhgqlke kngdvielky plncadptse rwfhghlsgk |
| 121 | eaeklltekg khgsflvres qshpgdfvls vrtgddkges ndgkskvthv mircqelkyd |
| 181 | vgggerfdsl tdlvehykkn pmvetlgtvl qlkqplnttr inaaeiesrv relsklaett |
| 241 | dkvkqgfwee fetlqqqeck llysrkegqr qenknknryk nilpfdhtrv vlhdgdpnep |
| 301 | vsdyinanii mpefetkcnn skpkksyiat qgclqntvnd fwrmvfqens rvivmttkev |
| 361 | ergkskcvky wpdeyalkey gvmrvrnvke saahdytlre lklskvgqgn tertvwqyhf |
| 421 | rtwpdhgvps dpggvldfle evhhkqesim dagpvvvhcr |

Included in Table 1 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein, but harbor one or more activating mutations or one or more inhibiting mutations to thereby, for example, activate a Jak kinase or inhibit a Jak kinase inhibitor.
Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein, but harbor one or more activating mutations or one or more inhibiting mutations to thereby, for example, activate a Jak kinase or inhibit a Jak kinase inhibitor.
Included in Table 1 are the well known SOCS family members other than SOCS1 and SOCS3, such as CIS and SOCS2 and SOCS4-7. In addition, any Jak kinase modulator, direct Jak kinase binding protein, cytokine, and cytokine receptor described herein is also included in Table 1. The nucleic acid and polypeptide descriptions provided above in the asterisked sections of Table 1 also apply.

### II. SUBJECTS

In one embodiment, the subject for whom predicted likelihood of efficacy of an anti-immune checkpoint inhibitor therapy is determined, is a mammal (e.g., mouse, rat, primate, non-human mammal, domestic animal such as dog, cat, cow, horse), and is preferably a human.

In another embodiment of the methods of the invention, the subject has not undergone treatment, such as chemotherapy, radiation therapy, targeted therapy, and/or anti-immune checkpoint inhibitor therapy. In still another embodiment, the subject has undergone treatment, such as chemotherapy, radiation therapy, targeted therapy, and/or anti-immune checkpoint inhibitor therapy.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the invention can be used to determine the responsiveness to anti-immune checkpoint inhibitor therapies of many different cancers in subjects such as those described above. In one embodiment, the cancers are solid tumors, such as lung cancer or lung cancer subtypes (e.g., squamous cell carcinoma), melanoma, and/or renal cell carcinoma. In another embodiment, the cancer is an epithelial cancer such as, but not limited to, brain cancer (e.g., glioblastomas) bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In still other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated.

### III. SAMPLE COLLECTION, PREPARATION AND SEPARATION

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to an anti-immune checkpoint inhibitor therapy, and/or evaluate a response to a combination anti-immune checkpoint inhibitor therapy. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., expression and/or activity of biomarkers to that of wild type biomarkers and expression and/or activity of a biomarker of interest normalized to that of a housekeeping gene).

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold or greater. In some embodiments, the fold change is less than about 1, less than about 5, less than about 10, less than about 20, less than about 30, less than about 40, or less than about 50. In other embodiments, the fold change in biomarker amount and/or activity measurement(s) compared to a predetermined level is more than about 1, more than about 5, more than about 10, more than about 20, more than about 30, more than about 40, or more than about 50.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., bronchoalevolar lavage fluid, amniotic fluid, aqueous humor, bile, blood and blood plasma, cerebrospinal fluid, cecrumen and earwax, cowpcr's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. IN another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH1 gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CLEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. BIOMARKER NUCLEIC ACIDS AND POLYPEPTIDES

One aspect of the invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonuclotides corresponding to all or a portion of a nucleic acid molecule of the invention can be prepared by standard synthetic techniques, e.g., using an automated DNA synthesizer.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonuclotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60%, 65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isolcucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine. N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in sin, such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol 11 or pol Ill promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-(641). The antisense nucleic acid molecule can also comprise a 2′-o-methylribonucleotide (Inoue et al., 1987, Nucleic Acids Res. 15:6131-6148) or a chimeric RNA-DNA analogue (Inouc et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 66):569-84: Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve. e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra: Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Rae. 24(17):3357-63). Alternatively, chimeric molecules can be synthesized with a 5′ DNA segment and a 3′ PNA segment (Peterser et al., 1975, Biorganic Med. Chem. Left. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sc. ISA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Re. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a biomarker protein. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence, immunoglobulin fusion protein, toxin, or other useful protein sequence. Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors for use in the invention can be designed for expression of a polypeptide corresponding to a marker of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, in Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989. EMBO. J. 8:729-733) and immunoglobulins (Banerji et al., 1983, Cell 33:729-740; Queen and Baltimore, 1983. Cell 33:741-748), neuron-specific promoters (e.g., the neurofilament promoter, Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine box promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook. et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. ANALYZING BIOMARKER NUCLEIC ACIDS AND POLYPEPTIDES

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 is predictive of poorer outcome of anti-immune checkpoint inhibitor treatment.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in sine hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonuclcotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible rumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well known in the art (see, e.g., U.S. Pat. Nos. 6,335,167; 6,197,501; 5,830.645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. IS 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A) RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see. e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610: strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nuclcotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to an anti-immune checkpoint inhibitor therapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker proteinantibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary fir all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to imnunobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify biomarkers.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophorectic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Protein expression and activity can also be assessed according to functional assays described further below.

### 3. Anti-Cancer Therapies and Combination Therapies

The efficacy of anti-immune checkpoint inhibitor therapy is predicted according to biomarker amount and/or activity associated with a cancer in a subject according to the methods described herein. In one embodiment, such anti-immune checkpoint inhibitor therapy or combinations of therapies (e.g., anti-PD-1, anti-PD-L1, anti-PD-L2, and anti-CTLA4 therapies) can be administered once a subject is indicated as being a likely responder to anti-immune checkpoint inhibitor therapy. In another embodiment, such anti-immune checkpoint inhibitor therapy can be avoided once a subject is indicated as not being a likely responder to anti-immune checkpoint inhibitor therapy and an alternative treatment regimen, such as targeted and/or untargeted anti-cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without anti-immune checkpoint inhibitor therapy.

The term “targeted therapy” refers to administration of agents that selectively interact with a chosen biomolecule to thereby treat cancer.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, the immunotherapy can comprise the use of a Jak kinase nucleic acid or polypeptide or other Jak kinase stimulator (e.g., a small molecule, an inhibitor of a Jak kinase inhibitor, and the like) in order to increase or overexpress Jak kinase activity. Without being bound by theory, it is believed that promoting Jak kinase activity, as opposed to the standard method in the art of inhibiting Jak kinase activity, increases expression of immune checkpoint inhibitory molecules thereby rendering cancer cells more susceptible to anti-immune checkpoint inhibitor therapy. Such Jak kinase stimulation can be transient (e.g., inducible at will for repeated exposure) or constitutive. Such Jak kinase stimulation can also be systemic (e.g., by generally administering Jak kinase-activating cytokine(s) or expressing a Jak kinase nucleic acid with a general promoter) or targeted (e.g., locally administering a Jak kinase activating cytokinc(s) or expressing a Jak kinase nucleic acid using a tissue-specific promoter).

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, carboplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, pemetrexed, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phcnanthridinone (Trevigen); benzamide (U.S. Pat. Re. 36,397); and NU 1025 (Bowman et al.). The mechanism of action is generally related to the ability of PARP inhibitors to bind PARP and decrease its activity. PARP catalyzes the conversion of .beta.-nicotinamide adenine dinuclcotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528: Wang Z Q. et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water, implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with anti-immune checkpoint inhibitor therapies may vary according to the particular anti-immune checkpoint inhibitor agent or combination thereof (e.g., Jak kinase stimulating agents in combination with inhibitors of PD-1, PD-L1, PD-L2, CTLA-4, and the like). An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

Any means for the introduction of a polynucleotide into mammals, human or non-human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Feigner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154. 1992. Other vehicles which can optionally be used include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant rctroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors.” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mannmmalian cells (Friedmann (1989) Science, 244:1275-1281; Ridgeway, 1988, supra; Baichwal and Sugden, 1986, supra; Coupar et al., 1988; Horwich t al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### 4. Clinical Efficacy

Clinical efficacy can be measured by any method known in the art. For example, the response to a therapy, such as anti-immune checkpoint inhibitor therapies, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint inhibitor therapeutic regimen is at least 25%, 30%, 35%, 40%, 45%, 50%, 55%, 60%, 65%, 70%, 75%, 80%, 85%, or more.

Additional criteria for evaluating the response to anti-immune checkpoint inhibitor therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-immune checkpoint inhibitor therapeutic regimen can be administered to a population of subjects and the outcome can be correlated to biomarker measurements that were determined prior to administration of any anti-immune checkpoint inhibitor therapy. The outcome measurement may be pathologic response to therapy given in the neoadjuvant setting. Alternatively, outcome measures, such as overall survival and disease-free survival can be monitored over a period of time for subjects following anti-immune checkpoint inhibitor therapy for whom biomarker measurement values are known. In certain embodiments, the same doses of anti-immune checkpoint inhibitor agents are administered to each subject. In related embodiments, the doses administered are standard doses known in the art for anti-immune checkpoint inhibitor agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an anti-immune checkpoint inhibitor therapy can be determined using methods such as those described in the Examples section.

### 5. Further Uses and Methods of the Present Invention

The compositions described herein can be used in a variety of diagnostic, prognostic, and therapeutic applications.

a. Screening Methods

One aspect of the present invention relates to screening assays, including non-cell based assays. In one embodiment, the assays provide a method for identifying whether a cancer is likely to respond to anti-immune checkpoint inhibitor therapy and/or whether an agent can inhibit the growth of or kill a cancer cell that is unlikely to respond to anti-immune checkpoint inhibitor therapy.

In one embodiment, the invention relates to assays for screening test agents which bind to, or modulate the biological activity of, at least one biomarker listed in Table 1. In one embodiment, a method for identifying such an agent entails determining the ability of the agent to modulate, e.g. inhibit, the at least one biomarker listed in Table 1.

In one embodiment, an assay is a cell-free or cell-based assay, comprising contacting at least one biomarker listed in Table 1, with a test agent, and determining the ability of the test agent to modulate (e.g. inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies of the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker metabolite and its natural binding partner can be accomplished by determining the ability of the test agent to modulate the activity of a polypeptide or other product that functions downstream or upstream of its position within the pathway (e.g., feedback loops).

The present invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

In some embodiments, detecting Jak kinase autophosphorylation is useful and methods for such detection are well known in the art. In an autophosphorylation assay, a test compound suspected of being a Jak kinase modulator is contacted or reacted with a suitable reaction mixture comprising JAK polypeptide as a source of tyrosine and/or serine kinase activity under conditions and for a time sufficient to allow phosphorylation of a tyrosine and/or serine residue. The tyrosine kinase reaction may be initiated in the presence of ATP or an analog thereof and Mn²⁺ or Mg²⁺ (e.g., as MnCl₂ or a mixture of divalent cations comprising Mn²⁺ or Mg²⁺), whereas the serine kinase reaction may be initiated in the presence of ATP and divalent cations, such as Mn²⁺ (e.g., as MnCl₂ or a mixture of divalent cations comprising Mn²⁺) or Mg²⁺ (e.g., as MgCl₂ or a mixture of divalent cations comprising Mg²⁺), or mixtures thereof. Subsequently, the presence or absence of autophosphorylated tyrosine and/or serine residues may be determined by standard methods known in the art. Such methods include, but are not limited to mass spectrometry, microscopy, spectroscopy, Western blotting, and immunoassays such as SPR, RIA, EIA, and ELISA, wherein phosphotyrosine or phosphoserine specific antibodies (including polyclonal, monoclonal, chimeric, and single chain antibodies, as well as FAb fragments) available in the art may be used. The antibody may be directly or indirectly labelled, for example, with a radiolabel, fluorescent label, luminescent label, or enzymatic label capable of producing a detectable signal.

The assay may comprise a step, wherein the level of serine and/or tyrosine phosphorylation in the presence of a test substance is compared to that in the absence of said test substance. In some embodiments, if the level of serine and/or tyrosine phosphorylation is increased as compared to the control (no test substance present), the test substance is a Jak kinase activator. In other embodiments, if the level of serine and/or tyrosine phosphorylation is decreased as compared to the control, the test substance is a Jak kinase inhibitor. IN still other embodiments, an inhibitor of autophosphorylation of the JH2 domain may act as an activator for JH1 domain catalytic activity and signaling, and in some specific embodiments the inhibitor may inhibit JH1 activity and signaling.

In other embodiments, the assay is based on the capability of a test compound to modulate the ability of a Jak kinase to bind a substrate or transphosphorylate tyrosine and/or serine residues of a substrate. The term “substrate” refers to a protein or a peptide which is acted on by the tyrosine and/or serine kinase activity of the Jak kinase such that it is phosphorylated on tyrosine and/or serine residues, respectively.

In a transphosphorylation assay, a test compound is contacted or reacted with a suitable reaction mixture comprising Yak polypeptide comprising a catalytically active JH2 domain as a source of tyrosine and/or serine kinase activity and a substrate. Suitable tyrosine and serine substrates are available in the art and include, but are not limited to, Poly-Gly-Tyr peptide. The kinase reaction is initiated in the presence of ATP and divalent cations such as Mn²⁺ or Mg²⁺ as described above. The reaction is carried out under conditions and for a time sufficient to allow phosphorylation of a tyrosine and/or serine residue. Subsequently, the presence or absence of phosphorylated tyrosine and/or serine residues in the substrate may be determined by standard methods known in the art as described above for autophosphorylation assays. Further, the assay may comprise a step, wherein the level of transphosphorylation in the presence of a test substance is compared to that in the absence of said test substance. If the level of serine and/or tyrosine transphosphorylation is increased as compared to the control (no test substance present), the test substance is an activator of Jak kinase activity. On the other hand, if the level of serine and/or tyrosine transphosphorylation is decreased as compared to the control, the test substance is an inhibitor of Jak kinase activity.

Jak kinase modulators can also be screened, identified, and characterized by employing calorimetric methods such as differential scanning calorimetry or fluorimetry, or isothermal titration calorimetry or fluorimetry, where the binding of the modulator is analysed with respect to a change in the melting temperature of the Jak kinase. Such methods are known to a person skilled in the art and include measurement of surface plasmon resonance or spectrocopical methods including fluorescence, UV/visible light, CD, NMR based methods and microscopy methods including atom force microscopy, as well as crystallography.

In cell-based assays, cells can be used that lack the specified biomarker of interest, such as a Jak kinase having an activating mutation. Receptor activation may be employed and the readout may be based on detection of tyrosine or serine phosphorylation in the context of Jak kinase autophosphorylation activation or Jak kinase catalysis of transphosphorylation or as activation of downstream signalling cascades/proteins, such as STAT transcription factors, PI-3K/Akt cascade, MAP kinase pathway, and the like. Furthermore, colony formation, cellular mobility, proliferation, other cellular functions can be used as a readout for the assays. In one embodiment, the expression of at least one immune checkpoint inhibitor is analyzed (e.g., PD-L expression).

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker listed in Table 1 in the context of a biological sample (e.g., blood, serum, cells, or tissue) to thereby determine whether an individual afflicted with a cancer is likely to respond to anti-immune checkpoint inhibitor therapy, whether in an original or recurrent cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers listed in Table 1.

Another aspect of the present invention pertains to monitoring the influence of agents (e.g., drugs, compounds, and small nucleic acid-based molecules) on the expression or activity of a biomarker listed in Table 1. These and other agents are described in further detail in the following sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to anti-immune checkpoint inhibitor therapy. In some embodiments, the present invention is useful for classifying a sample (e.g., from a subject) as associated with or at risk for responding to or not responding to anti-immune checkpoint inhibitor therapy using a statistical algorithm and/or empirical data (e.g., the amount or activity of a biomarker listed in Table 1).

An exemplary method for detecting the amount or activity of a biomarker listed in Table 1, and thus useful for classifying whether a sample is likely or unlikely to respond to anti-immune checkpoint inhibitor therapy involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely anti-immune checkpoint inhibitor therapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decisionclassification trees such as random forests, classification and regression trees (C&RT), boosted trees, etc.), Probably Approximately Correct (PAC) learning, connectionist learning (e.g., neural networks (NN), artificial neural networks (ANN), ncuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown enviromnent, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to anti-immune checkpoint inhibitor therapy), a biological sample from the subject during remission, or a biological sample from the subject during treatment for developing a cancer progressing despite anti-immune checkpoint inhibitor therapy.

d. Prognostic Assays

The diagnostic methods described herein can furthermore be utilized to identify subjects having or at risk of developing a cancer that is likely or unlikely to be responsive to anti-immune checkpoint inhibitor therapy. The assays described herein, such as the preceding diagnostic assays or the following assays, can be utilized to identify a subject having or at risk of developing a disorder associated with a misregulation of the amount or activity of at least one biomarker described in Table 1, such as in cancer. Alternatively, the prognostic assays can be utilized to identify a subject having or at risk for developing a disorder associated with a misregulation of the at least one biomarker described in Table 1, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The compositions described herein (including dual binding antibodies and derivatives and conjugates thereof) can be used in a variety of in vitro and in vivo therapeutic applications using the formulations and/or combinations described herein. In one embodiment, anti-immune checkpoint inhibitor agents can be used to treat cancers determined to be responsive thereto. For example, antibodies that block the interaction between PD-L1. PD-L2, and/or CTLA-4 and their receptors (e.g., PD-L1 binding to PD-1, PD-L2 binding to PD-1, and the like) can be used to treat cancer in subjects identified as likely responding thereto.

### 6. Pharmaceutical Compositions

In another aspect, the present invention provides pharmaceutically acceptable compositions which comprise a therapeutically-effective amount of an agent that modulates biomarker expression and/or activity (e.g., increases Jak kinase activity and/or decreases the activity of Jak kinase inhibitors), one or more anti-immune checkpoint inhibitors, or a combination thereof, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes: (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates biomarker expression and/or activity, or expression and/or activity of the complex, or composition comprising an agent that modulates biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water, (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates biomarker expression and/or activity, or expression and/or activity of the complex encompassed by the invention. These salts can be prepared in situ during the final isolation and purification of the agents, or by separately reacting a purified agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates biomarker expression and/or activity, or expression and/or activity of the complex. These salts can likewise be prepared in situ during the final isolation and purification of the agents, or by separately reacting the purified agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylendiamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates biomarker expression and/or activity, with the carrier and, optionally, one or more accessory ingredients. In general, the formulations are prepared by uniformly and intimately bringing into association a agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia: (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or metabolite standards). A kit may additionally include buffers and other reagents recognized for use in a method of the disclosed invention. Non-limiting examples include agents to reduce non-specific binding, such as a carrier protein or a detergent.

Other embodiments of the present invention are described in the following Examples. The present invention is further illustrated by the following examples which should not be construed as further limiting.

### EXAMPLES

### Example 1: Materials and Methods for Examples 2-4

a. Subject

The programmed cell death-1 (PD-1) protein is a co-inhibitory receptor that restrains immune signaling by inhibiting T cell function. Tumors that express its major inducible ligand, PD-L1, evade immunosurveillance by engaging the PD-1 immune checkpoint (Dong et al. (2002) Nat. Med. 8:793-800; Freeman et al. (2000) J. Exp. Med. 192:1027-1034). In preclinical models, blockade of PD-L1 interaction with PD-1 promotes immune-mediated antitumor activity (Iwai et al. (2002) Proc. Natl. Acad. Sci. U.S.A. 99:12293-12297). Clinical trials of PD-1 and PD-L1 inhibitors have uncovered durable tumor regression in a subset of patients with a variety of aggressive cancers (Brahmer et al. (2012) New Engl. J. Med. 366:2455-2465; Topalian et al. (2012) New Engl. J. Med. 366:2443-2454; Ansell et al. (2015) New Engl. J. Med. 372:311-319; Powles et al. (2014) Nature 515:558-562). Although studies have suggested that tumors PD-L expression in tumors or tumor infiltrating immune cells (Herbst et al. (2014) Nature 515:563-567) appear more likely to respond to immune checkpoint inhibition, the specific determinants of this enhanced responsiveness remain incompletely characterized.

Identifying genomic mechanisms of inhibitor sensitivity may inform patient selection for agents targeting immune checkpoints and suggest approaches to enhance their efficacy in otherwise resistant patients. Comprehensive genomic profiling of exceptional responders has revealed the genomic mechanism of extraordinary response to targeted therapies (Iyer et al. (2012) Science 338:221; Al-Ahmadie et al. (2014) Cancer Disc. 4:1014-1021; Imielinski et al. (2014) J. Clin. Invest. 124:1582-1586; Wagle et al. (2014) New Engl. J. Med. 371:1426-1433; Wagle et al. (2014) Cancer Disc. 4:546-553), but has not yet been applied to immunotherapies.

A 57 year old male with an 40 pack-year smoking history presented with left shoulder discomfort. Magnetic resonance imaging (MRI) revealed a 1×1.4×2 cm lytic lesion in the left humeral head. A computed tomography (CT)-guided biopsy of this lesion was obtained, which demonstrated CK7 and TTF-1 positive adenocarcinoma suggestive of primary lung origin and lung cancer. Subsequent CT of the chest demonstrated a 4×3.3×2 cm mass in the left apex of the lung. PET confirmed that this mass was FDG-avid, and there was left paratracheal lymphadenopathy and lytic metastasis in the proximal left humerus. Brain MRI revealed four small solid enhancing lesions consistent with additional metastatic disease. Thus, the patient was diagnosed with Stage IV metastatic lung adenocarcinoma.

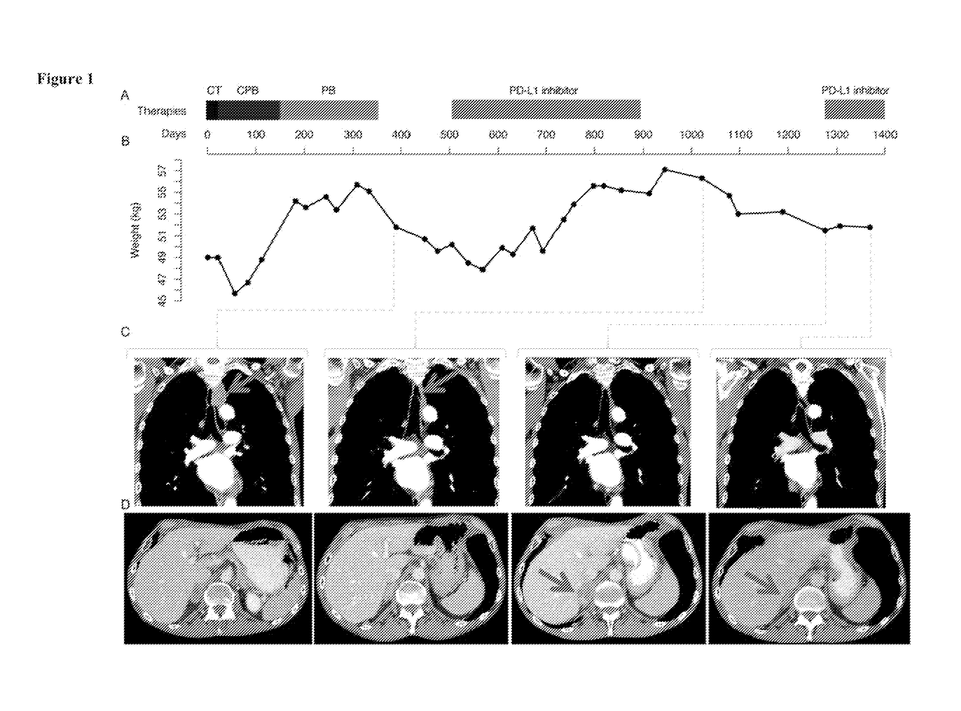

The patient received palliative radiation therapy to the left shoulder and whole brain radiation therapy, followed by a single cycle of carboplatin and paclitaxel, which he tolerated poorly (FIG. 1A). He then developed a perirectal abscess and was switched to dose-reduced carboplatin and pemetrexed, together with bevacizumab for three additional cycles and was transitioned to maintenance pemetrexed and bevacizumab.

After 8 months of maintenance therapy, the restaging CT scans demonstrated growth of a left adrenal mass. Laparoscopic left adrenalectomy was performed for palliation of severe flank pain and to obtain tissue for further genetic and immunohistochemical (IHC) testing. Initial clinical testing for oncogenic alterations revealed non-mutated wild-type EGFR, KRAS, and ALK. Three months later the patient developed a new right adrenal mass and worsening mediastinal lymphadenopathy (recurrence of the left paratracheal lymphadenopathy). Hospice was considered in the setting of worsening pain and weight loss (FIG. 1B). Imnununohistochemistry (IHC) performed on the excised left adrenal tumor demonstrated PD-L1 reactivity, prompting enrollment on Dana-Farber/Harvard Cancer Center (DF/HCC) clinical trial 11-314, a phase 1 study of MPDL3280A, an engineered anti-PDL1 antibody.

The patient provided written informed consent for research biopsies, genomic profiling, and sequencing of tumor and normal DNA, as approved by the Dana-Farber/Harvard Cancer Center Institutional Review Board (DF/HCC Protocol 11-104).

The patient also consented to enroll on a phase 1 multicenter open-label dose-escalation study to evaluate the safety, tolerability, and pharmacokinetics of MPDL3280A (DF/HCC Protocol 11-314; NCT#01375842). He met all eligibility criteria, which included histologically or cytologically documented incurable or metastatic solid malignancy that failed to respond to available standard therapy; there was measurable disease by RECIST; ECOG performance status was 1; brain metastasis had been treated and was stable; laboratory testing was within protocol parameters; there was no history of autoimmune disease or evidence of active pneumonitis; no prior anti-CTLA4, anti-PD1, or anti-PD-L1 therapy; no intercurrent infection or illness; no history of hypersensitivity to chimeric or humanized antibodies; and steroids were held for 4 weeks prior to starting. As part of the inclusion criteria, his tumor was also tested for PD-L1 expression and found to be positive. The drug was administered as a single agent at a dose of 20 mg/kg every 3 weeks. After cycle 2 was delayed for 3 weeks due to a series of falls and hospital admissions, he gradually improved and received 16 cycles total. Therapy was tolerated well other than a mild flare of his underlying chronic obstructive pulmonary disease that responded to an albuterol inhaler daily. Tumor response and evaluation was performed by physical examination and serial chest/abdomenipelvic CT scans, and target lesions were evaluated using RECIST criteria. Following treatment discontinuation at 1 year, the patient has continued to be monitored every 3 months by examination and CT scans.

Thus, after a temporary decline, the patient subsequently received sixteen cycles of MPDL3280A over a one-year period. He achieved a partial response by RECIST criteria (FIG. 1C), and, significantly, he experienced complete resolutions of his symptoms, discontinuation of all narcotic pain medication, and return to pre-diagnosis body weight. He completed one year of therapy per protocol and remained without evidence of disease progression for an additional 12 months. At this point, he began to lose weight again and developed regrowth of the right adrenal mass (FIG. 1D), leading to re-initiation of MPDL3280A therapy. Restaging scans after another 3 months of MPDL3280A showed rapid improvement of the right adrenal lesion (FIG. 1D).

Given his extraordinary and repeated response to PD-L1 immune checkpoint blockade, comprehensive genomic profiling of the patient's tumor and germline sample was performed. A blood sample was obtained from the patient for germline testing after the complete response was identified, and whole blood was stored at −80° C. until DNA extraction was performed. Tumor specimens for PD-L1 immunohistochemistry and blood samples were obtained from additional patients consented through DF/HCC Protocol 11-104. Normal blood donor samples were accessed through DF/HCC Protocol 10-145.

b. Histology and Staining

Histopathologic analysis of the surgically resected left adrenal gland revealed metastatic adenocarcinoma, consistent with metastasis from the patient's lung primary. Surgical resection margins were negative for tumor and the tumor was confined by the adrenal capsule. The tumor showed approximately 10% necrosis and fibrosis consistent with partial treatment effect. Immunohistochemistry (IHC) for tumor PD-L1 expression was performed as described in Chen et al. (2013) Clin. Cancer Res. 19:3462-3473, Chouciri et al. (2014) Anna. Oncol. 25:2178-2184, and Shi et al. (2014) Amer. J. Surg. Pathol. 38:1715-1723. In brief, PD-L1 (9A11, 1:125) was performed on the Leica BOND III platform using Bond's Polymer Refine Detection kit. Heat-induced antigen retrieval was performed in Bond ER2 solution for 30 minutes online. Sections were incubated at room temperature in primary antibody for 120 minutes at room temperature. Upon staining completion, slides were dehydrated and coverslipped offline. INC interpretation was blinded to JAK3 V722I or P132T status when assessing PD-L1 stain. A randomly control set of 9 lung cancers identified by an independent pathologist was stained in parallel to determine relative enrichment. Scoring was performed by measuring the average number of positive cells in a given sample and the average intensity of staining (0=no staining, 1+=weak, 2+=moderate, 3+=intense positive staining, with all positive staining considered over background). Determination of statistically significant differences between the groups was performed by calculating an adjusted expression (H) score (% positive cells×staining intensity) (Chouciri et al. (2014) Annal. Oncol. 25:2178-2184; Azuma et al. (2014) Annal. Oncol. 25:1935-1940). Macrophage cells were identified through morphologic determination in intratumoral or alveolar spaces.

c. Site-Directed Sequencing

Standard techniques were utilized to extract genomic DNA from tumor within the left adrenalectomy specimen and from blood. Initial sequencing of tumor DNA was performed using the OncoMap assay, which detects mutations in 41 cancer genes at 471 different loci using multiplex PCR to amplify the region containing the variant of interest (MacConail et al. (2009) PLoS One 4: e7887). Following primer extension of the allele-specific DNA products, DNA analyses were measured using chip-based mass spectrometry (Sequenom MassARRAY 4). Additional tumor samples that harbored variants of interest in JAK3 were identified through the Dana-Farber Cancer Institute (DFCI) PROFILE project using the OncoMap assay, which included V722I and P132T variants. All lung cancer tumor samples from patients who provided written informed consent (DF/HCC Protocol 11-104) with adequate tumor tissue who underwent OncoMap assay testing done between 2010 and 2013 were included in the query. The OncDRS clinical-genomics database system that links genomic data from PROFILE with salient clinical annotations was utilized to provide a listing of patients with lung cancers who harbored JAK3 variants of interest, and all such patients with available tumor samples were studied with immunohistochemistry for PD-L1, as described above. These samples were then obtained for additional analyses, including histology and staining for PD-L1 described above.

d. Whole Exome Sequencing

In summary genomic DNA was sheared, end repaired, ligated with barcoded Illumina sequencing adapters, amplified, size selected and subjected to in solution hybrid capture using the Agilent SureSelect® Human All Exon v2.0 bait set (19, 20). Resulting exome Illumina sequencing libraries were then qPCR quantified, pooled, and sequenced with 76 base paired-end reads using HiSeq2500 sequencers (Illumina, USA). Raw BAM files are deposited in phs000694.v1.p1.

Sequence Data Processing and Quality Control:

Exome sequence data processing was performed using established analytical pipelines at the Broad Institute. Tumor and normal sequences were aligned to the hg19 human genome build from Illumina sequencing reads using the Picard pipeline (available on the World Wide Web at picard.sourceforge.net). The BAM was uploaded into Firehose (available on the World Wide Web at www.broadinstitute.org/cancer/cga/Firehose), which manages input and output files. Comparison of the origin for tumor and normal genotypes was performed to assess fingerprinting concordance, and cross-contamination of samples was estimated using ContEst (Cibulskis et al. (2011) Bioinform. 27:2601-2602).

Alteration Identification:

MuTect (Cibulskis et al. (2013) Nat. Biotech. 31:213-219) was applied to identify somatic single-nucleotide variants. DNA oxidation artifacts induced during sequencing were computationally removed using a filter-based method (Costello et al. (2013) Nucl. Acids Res. 41:e67). Indelocator (available on the World Wide Web at broadinstitute.org/cancer/cga/indelocator) was applied to identify small insertions or deletions. Annotation of identified variants was done using Oncotator (available on the World Wide Web at broadinstitute.org/cancer/cgatoncotator). Copy ratios were calculated for each captured target by dividing the tumor coverage by the median coverage obtained in a set of reference normal samples. The resulting copy ratios were segmented using the circular binary segmentation algorithm. Genes in copy ratio regions with segment means of greater than log 2(4) were evaluated for focal amplifications, and genes in regions with segment means of less than log₂(0.5) were evaluated for deletions. Genome wide copy-ratios were estimated from whole-exome sequencing (WES) data by comparison of the observed depth of coverage at each exon to that achieved in normal samples. Allelic copy-ratios were then estimated by analysis of allelic fractions for all heterozygous SNPs identified in the paired normal sample. Purity and ploidy evaluations to derive absolute copy number were made using ABSOLUTE (Carter et al. (2012) Nat. Biotech. 30:413-421). Heuristic analysis of all somatic alterations was performed using PHIAL (Van Allen et al. (2014) Nat. Med. 20:682-688). Somatic alterations were manually reviewed using Integrated Genomics Viewer (Robinson et al. (2011) Nat. Biotech. 29:24-26; Thorvaldsdottir et al. (2013) Brief. Bioinform. 14:178-92).

e. Experimental analysis

Cell Culture:

293T and Calu-1 cells were maintained in DMEM and RPMI 1640 respectively, with 10% FBS. Beas-2B was maintained in keratinocyte SFM, supplemented with human recombinant EGF and BPE (Gibco). All media were supplemented with 1% penicillin/streptomycin.

Plasmids, Immunoblotting, Flow Cytometry:

JAK3 mutant alleles were generated from pLX304-JAK3-WT (Broad Institute, TRC) using the Quikchange® Lightning Site-Directed Mutagenesis Kit (Agilent Technologies), and transferred into the pLX304 vector using the Gateway LR Clonase II enzyme mix from Life Technologies. 293T cells were transfected using X-tremeGENE® HP DNA Transfection Reagent (Roche) with pLX304 EGFP, JAK3-WT or JAK3 constructs as described in Zhu et al. (2014) Cancer Discov. 4:452-465. Lysates were harvested after 48 hours and immunoblotting was also performed according to a standard protocol (Zhu et al. (2014) Cancer Discov. 4:452-465). JAK3 (#8827) and Y980/981 pJAK3 (#5031) antibodies were from Cell Signaling Technologies. Bcas-2B and Calu-1 cells were infected with lentivirus generated from pLX304 empty control or the same JAK3 constructs and selected in blasticidin to derive stably infected cell lines as described in Zhu et al. (2014) Cancer Discov. 4:452-465. Flow cytometry for PD-L1 expression was performed 72 hours after plating as described in Akbay et al. (2013) Cancer Discov. 3:1355-1363. In brief, cells were stained with an anti-PD-L1 antibody (29E.2A3) or isotype control antibody, and levels of PD-L1 expression were quantified using a BD FACSCanto II® flow cytometer equipped with Diva software (BD Biosciences). Levels were compared with isotype control antibodies. PD-L1 mean fluorescence intensity (MFI) was normalized to isotype control. For EGF stimulation cells were incubated with EGF (50 ng/ml) for 72 h prior to FACS analysis.

PBMC Isolation and Stimulation:

Peripheral blood mononuclear cells (PBMCs) from patients identified as having JAK3 V722I or P132T variants (see sequencing section above) and healthy donors were isolated from fresh blood and platelet-depleted blood collars, respectively, by Ficoll method. PBMCs were plated at a density of 7.5×10⁵ cells/ml in a 48-well plate and stimulated with 250 ng/ml IFNγ (PBL Interferon Source). No stimulation controls were set-up for each donor. At 48 h post-stimulation, cells were treated with 2 mM EDTA and collected for flow cytometry to assess for PD-L1 expression on CD14+ myeloid cells. Cells were stained with Live/Dead yellow viability dye (Invitrogen), as well as antibodies (BD Biosciences) against CD14 (M5E2), CD11b (ICRF44), and PD-L1 (MIH1) for 30 min at 4 C, and then fixed in BD Cytofix® buffer prior to analyses on BD LSR Fortessa SORT® HTS flow cytometer. Given the need to compare the difference between two means in relation to the variation in the data, a t-test was used to compare PD-L1 inducibility between V722I and non-V722I monocytes.

T Cell Proliferation Assay:

PBMCs were isolated from patient blood samples immediately pre- and 1 h post-treatment with MPDL3280A, as well as from a normal donor by Ficoll and plated in a density of 4×10⁶ cells/ml to a 96-well plate. After 2 h at 37° C. non-adherent cells (lymphocyte portion) were removed by pipetting and remaining adherent cells (monocyte portion) were cultured with or without 250 ng/ml IFNγ. At 24 h, non-adherent lymphocytes were labeled with CFSE. Monocytes (IFNγ treated or untreated) were harvested by 5 mM EDTA treatment and resuspended in fresh medium. Both lymphocytes and monocytes were then plated to a 96-well plate pre-coated with 10 μg/ml OKT3 antibody. Proliferation of CD4+ T cells and CD8+ T cells were monitored at 72 h post-stimulation by CSFE dilution.

### Example 2: Genomic Profiling Identified Multiple Mutations in JAK3

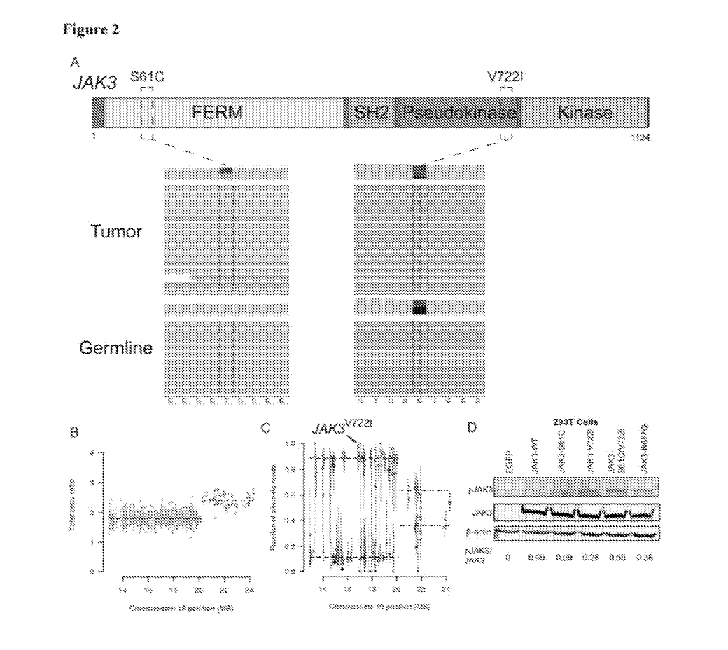

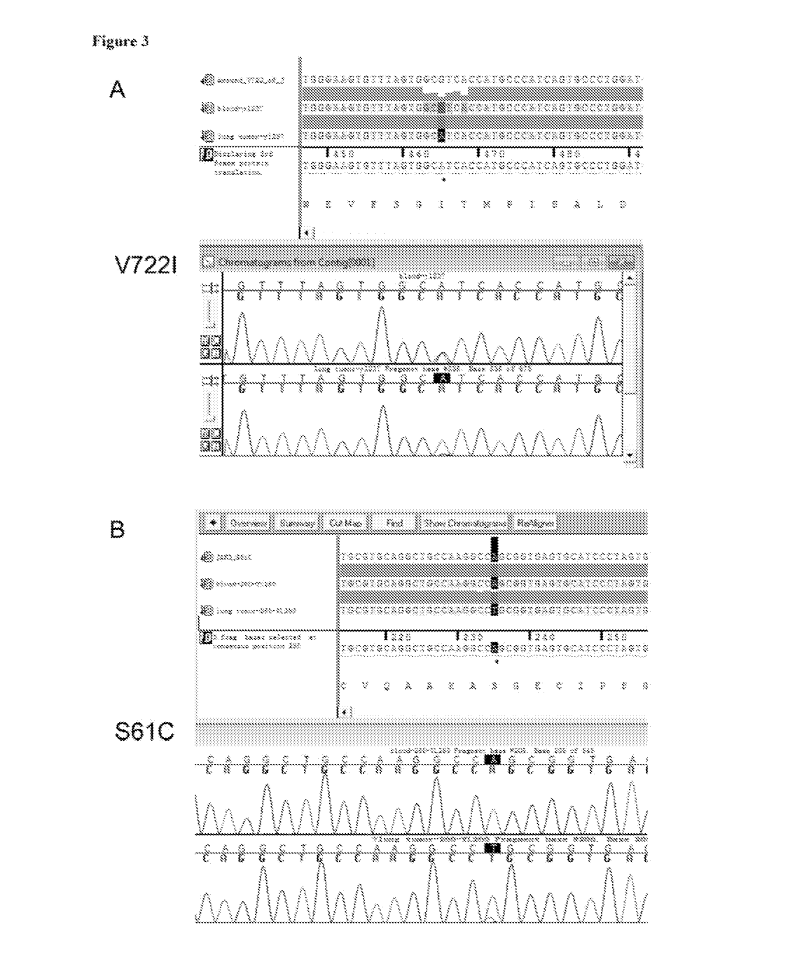

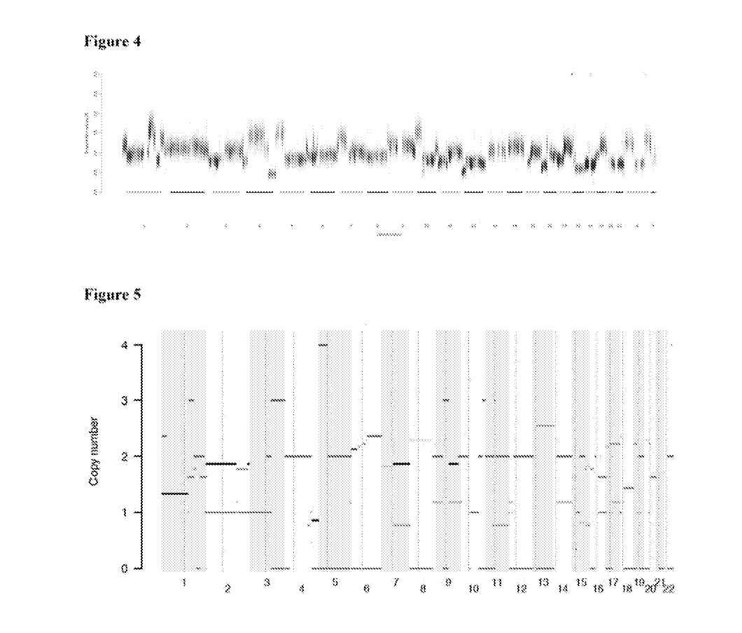

The only variant observed in the mass spectrometric genotyping panel was JAK3^{V722I}. This variant, which is located in the pseudokinase or JH2 domain of JAK3 (FIGS. 2A and 3A), has been described and functionally characterized as an activating allele in patients with acute megakaryocytic leukemia (Walters et al. (2006) Cancer Cell 10:65-75), acute lymphoblastic leukemia (Yin et al. (2015) Leuk. Lymph. epub 01/21/15 doi:10.3109110428194.2014.957204), and extranodal nasal-type natural killer cell lymphoma (Bouchekioua et al. (2014) Leukem. 28:338-348). JAK3^{V722I} has also been identified in peripheral blood from normal subjects (Riera et al. (2011) Leukem. Lymph. 52:1742-1750), and the population frequency of this hyperactive germline variant is approximately 1% (Exome Variant Server (cited 2014 June] available on the World Wide Web at evs.gs.washington.edu/EVS/).

Whereas tumor WES revealed neutral copy of the JAK3 locus on chromosome 19 in aggregate (FIGS. 2B and 4), allele-specific copy number segmentation demonstrated near complete conversion to the mutant allele in this region (FIGS. 2C and 5). The allelic fraction of the JAK3^{V722I} locus was 0.88 (131/149 reads) in the tumor and 0.47 (90/190 reads) in the germline sample, consistent with homozygosity of the JAK3^{V722I} allele in the tumor sample (which was 76% pure) and similar to the selection that occurs for activating JAK2^{V617F} alleles in myeloproliferative neoplasms (MPNs) (Gonzalez et al. (2014) PLoS One 9:e86401).

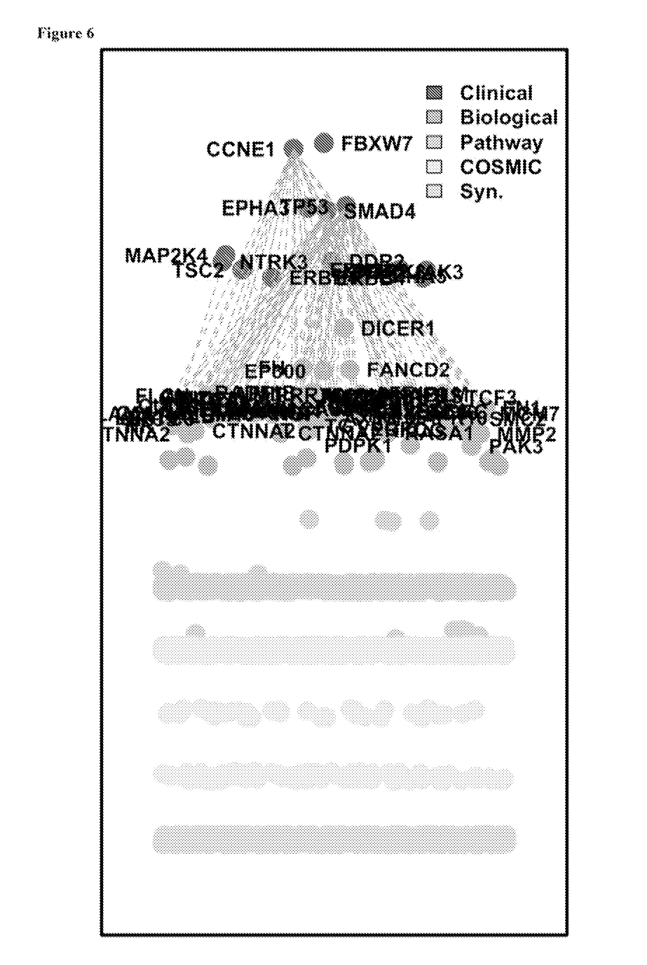

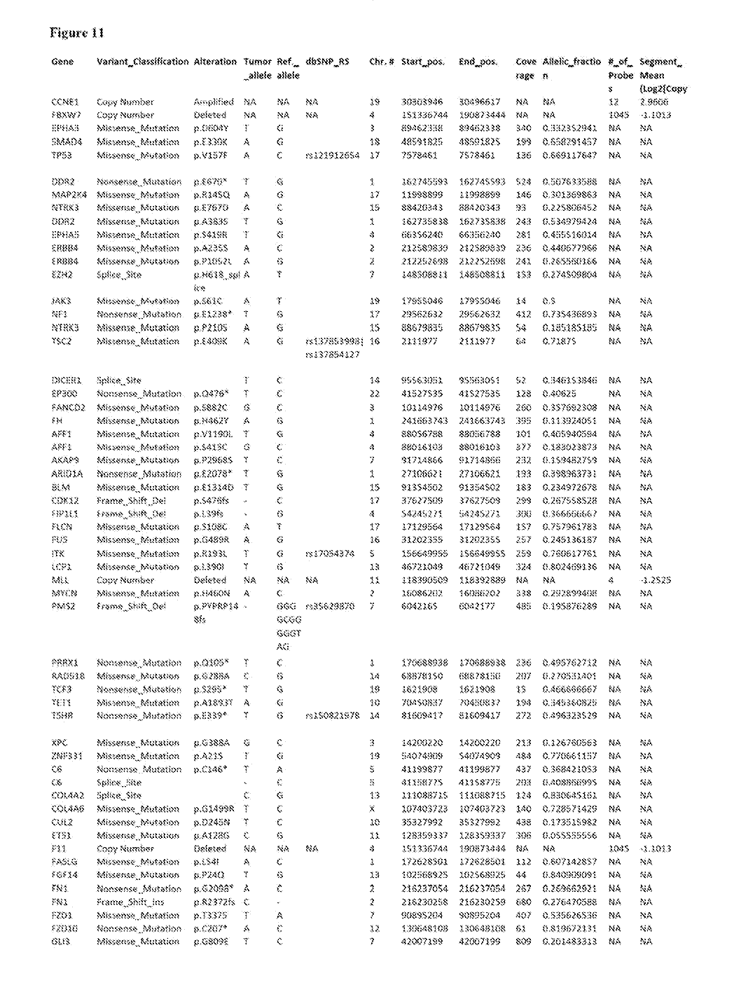

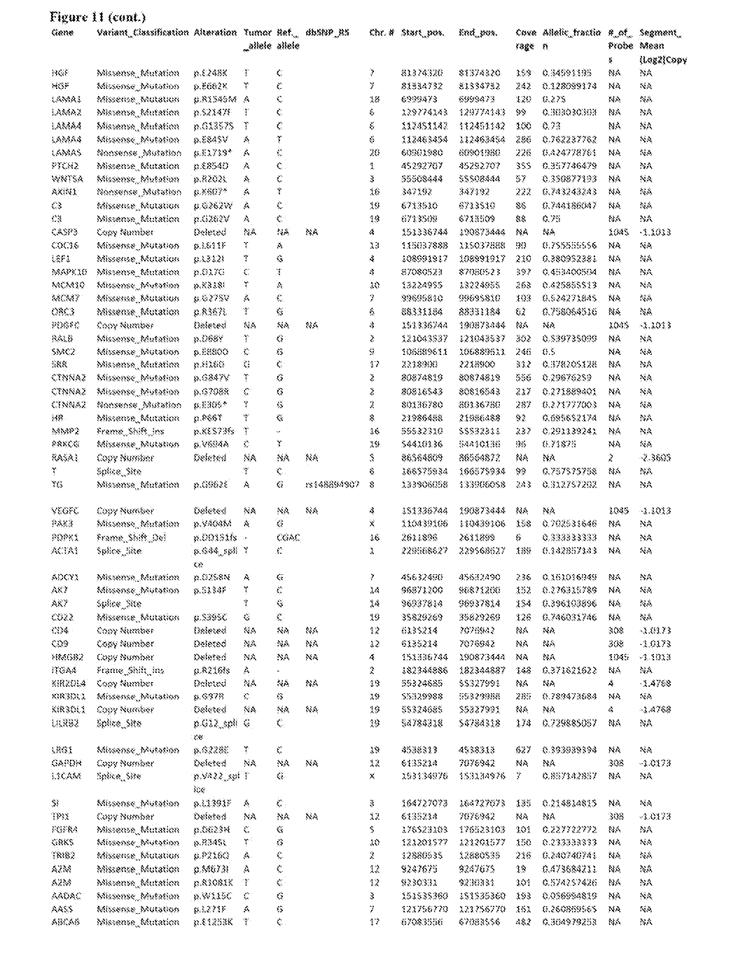

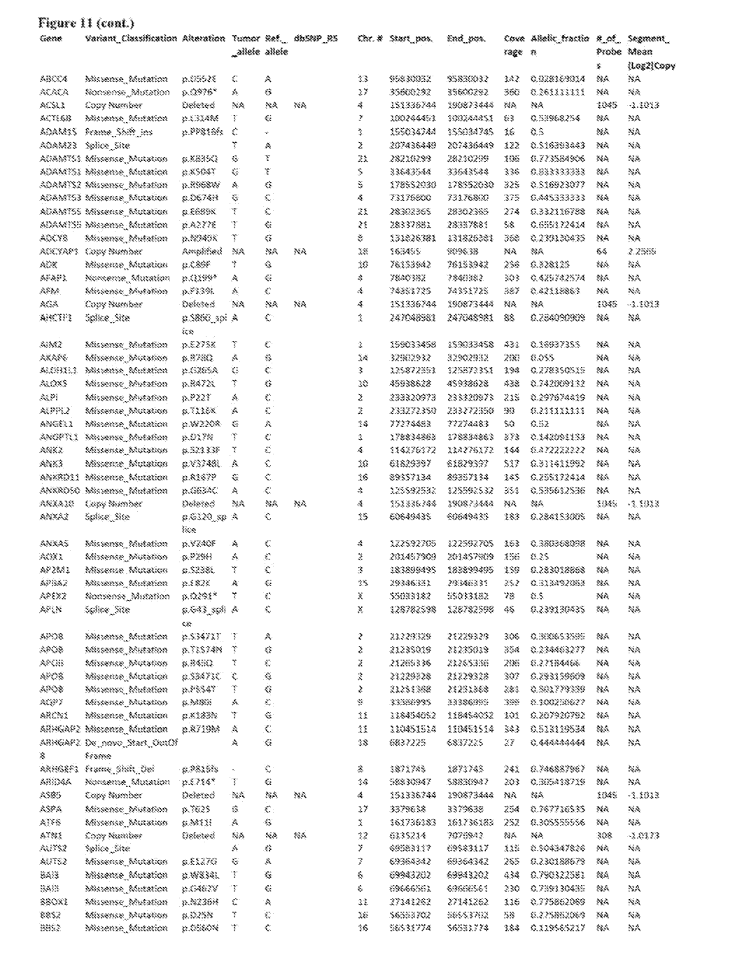

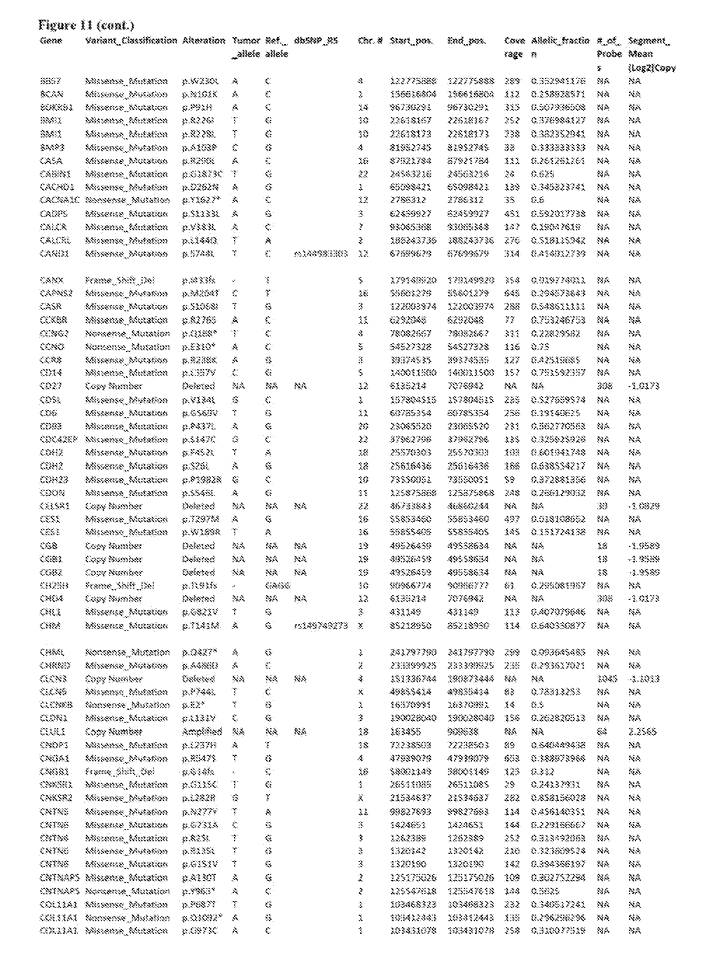

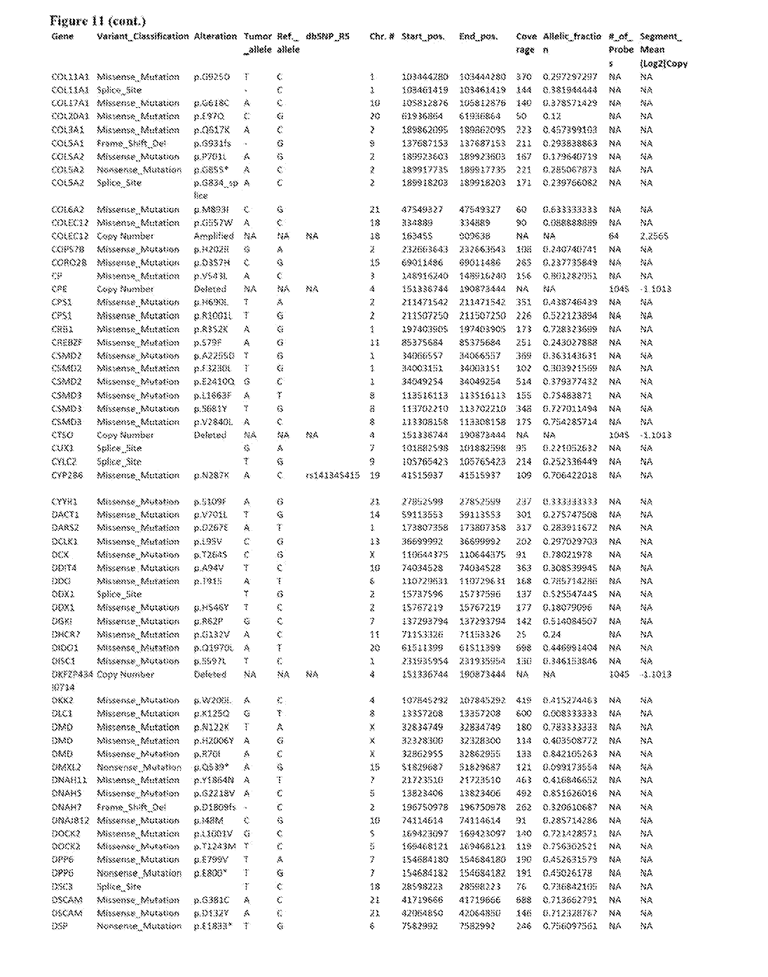

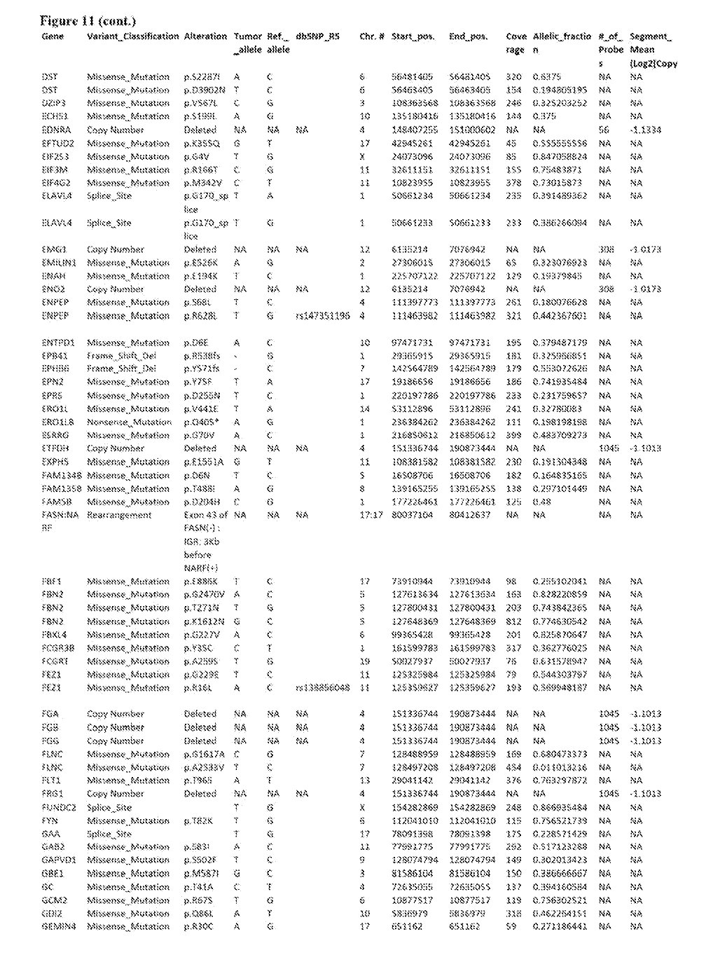

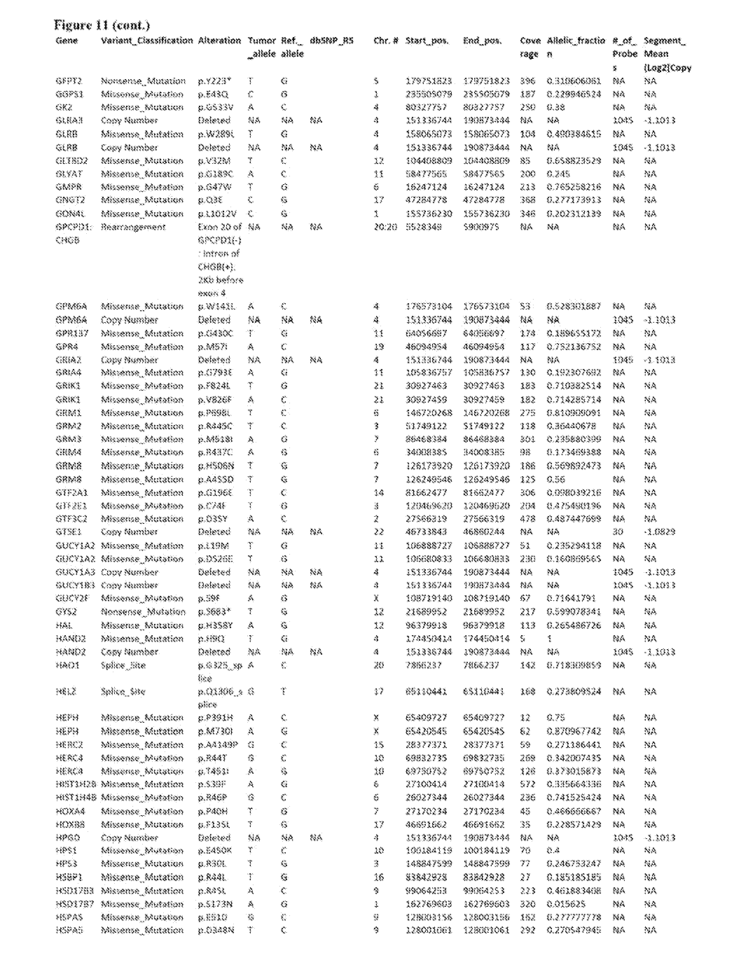

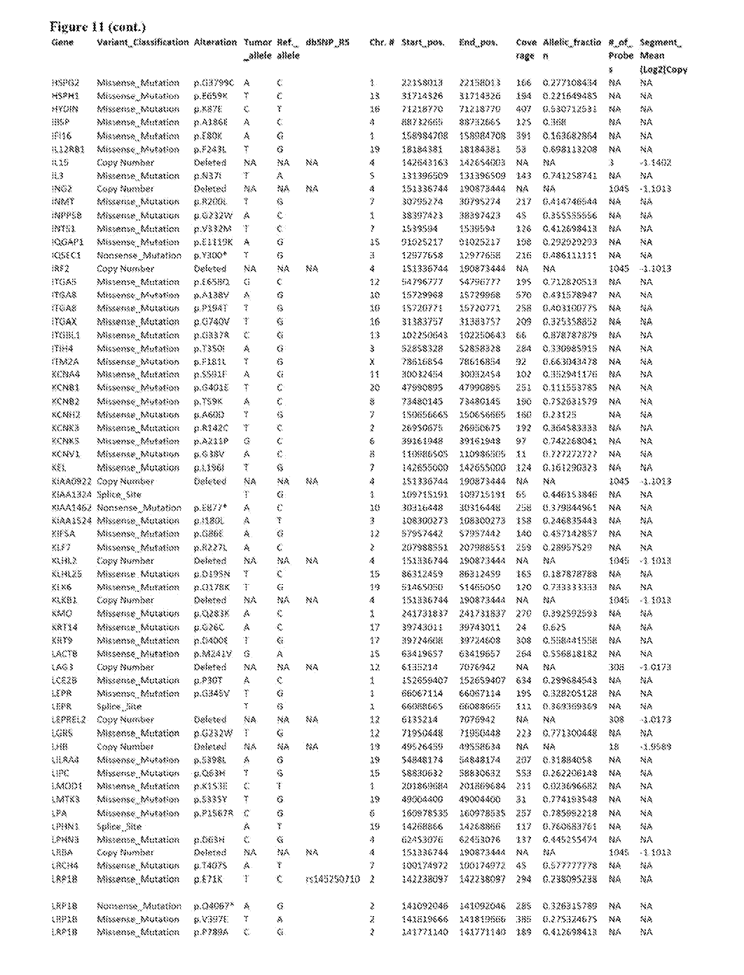

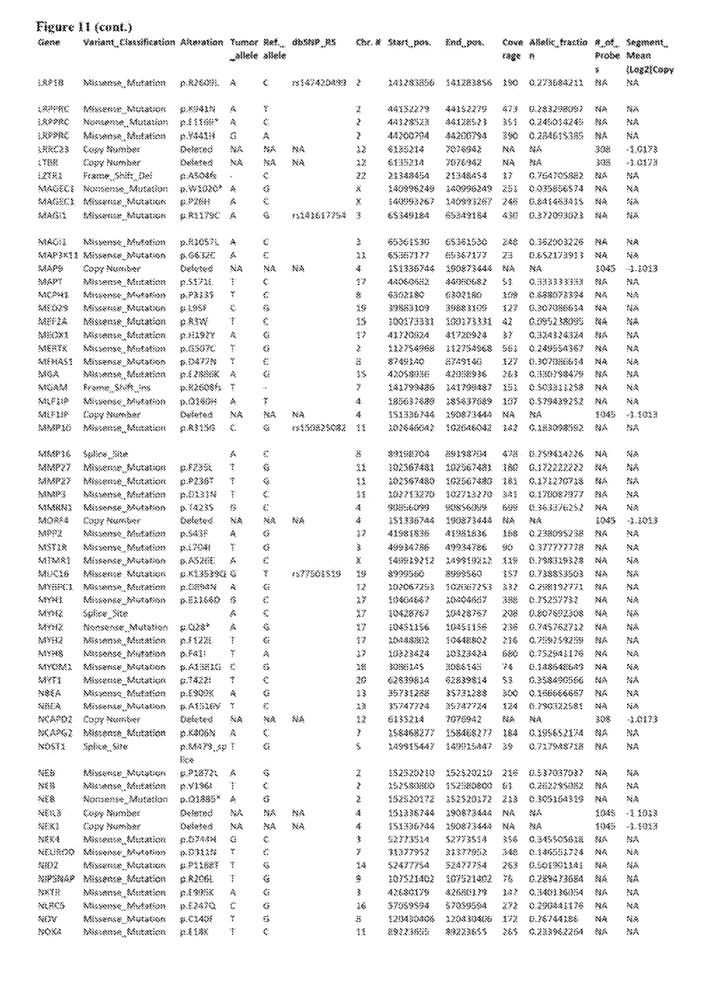

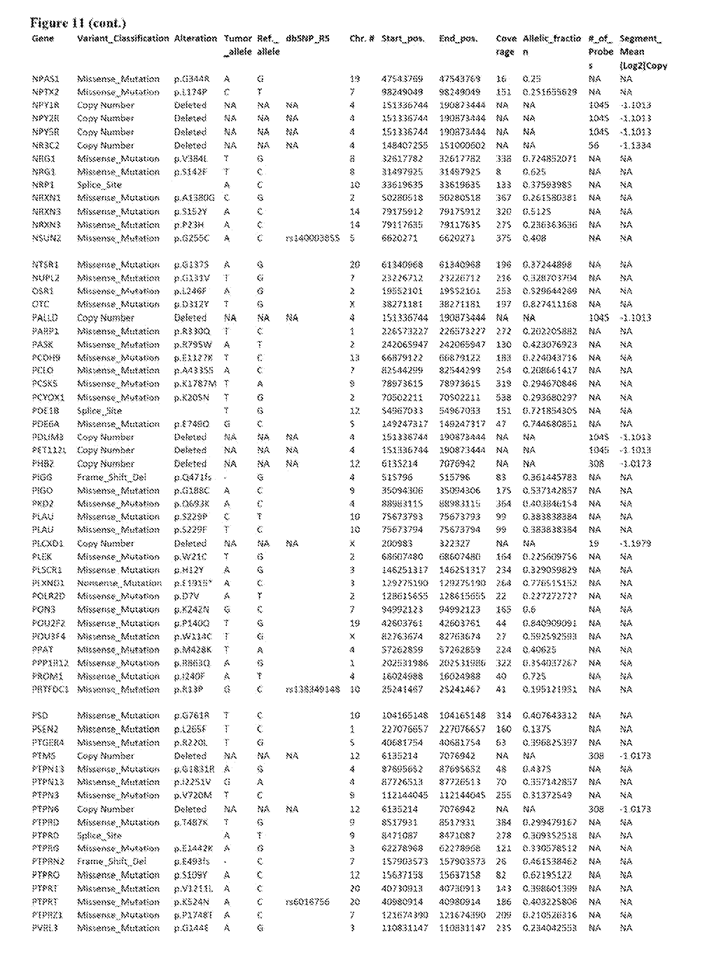

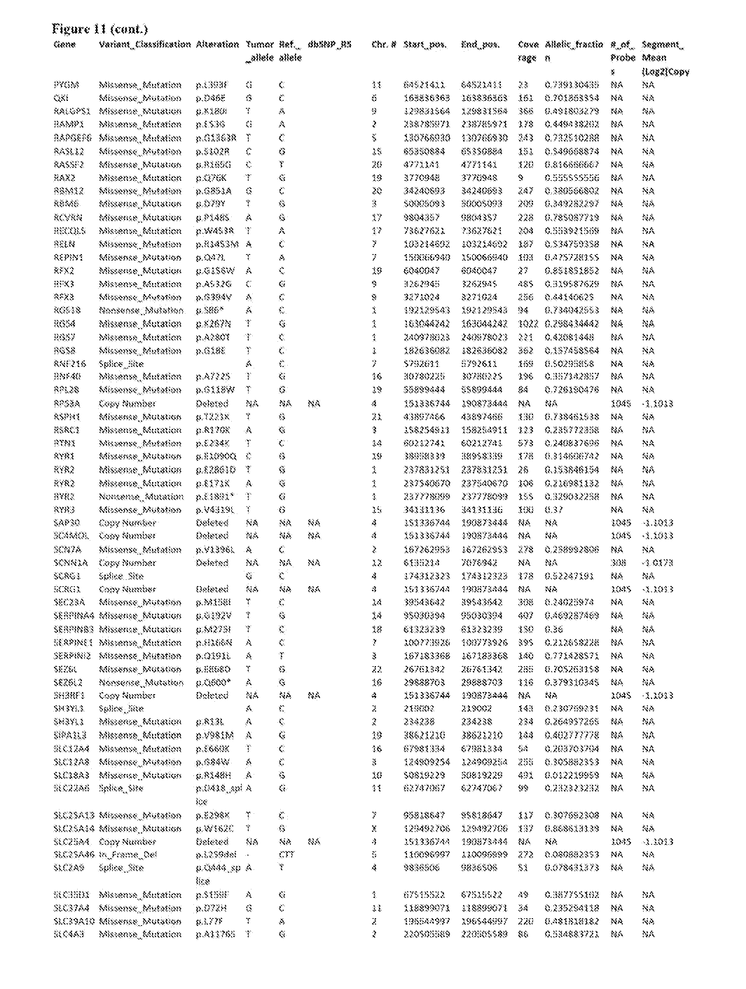

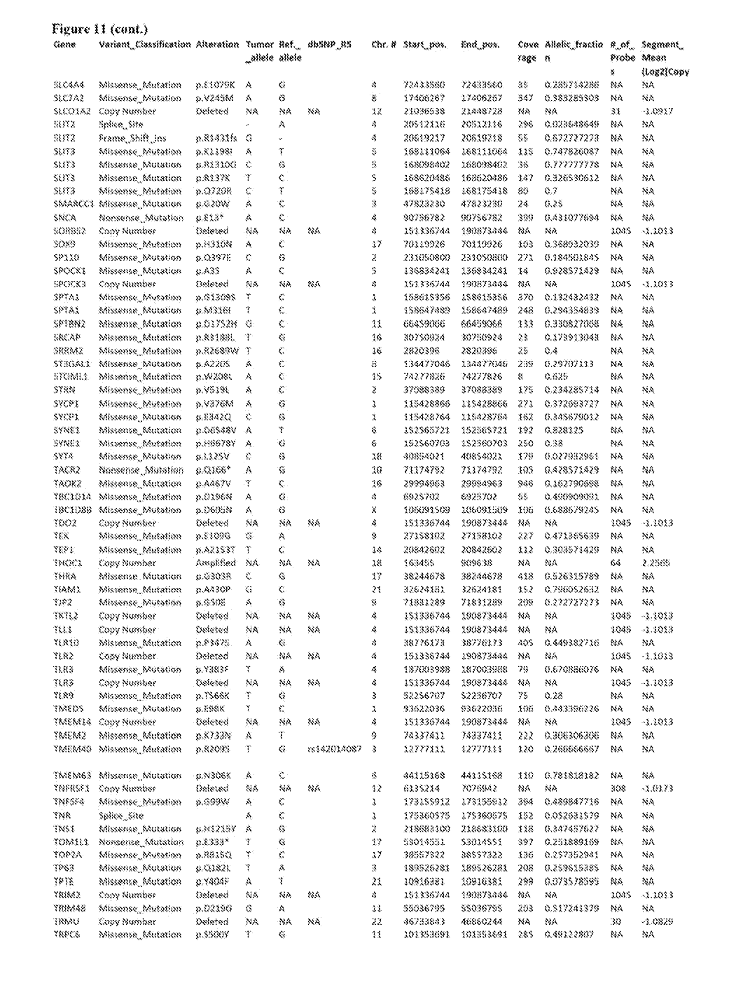

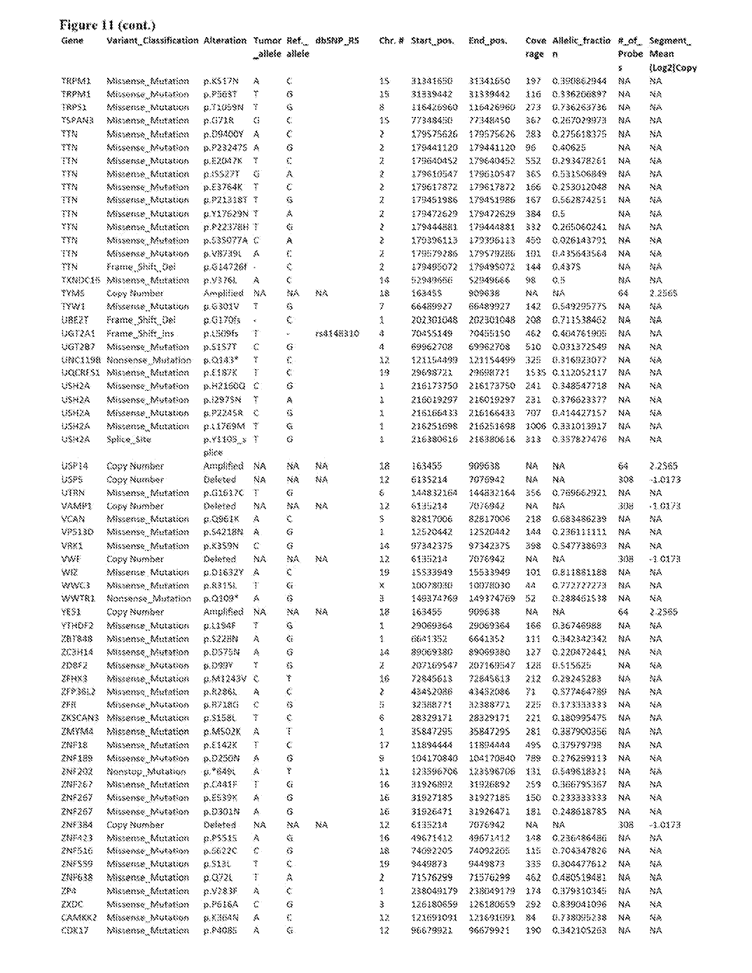

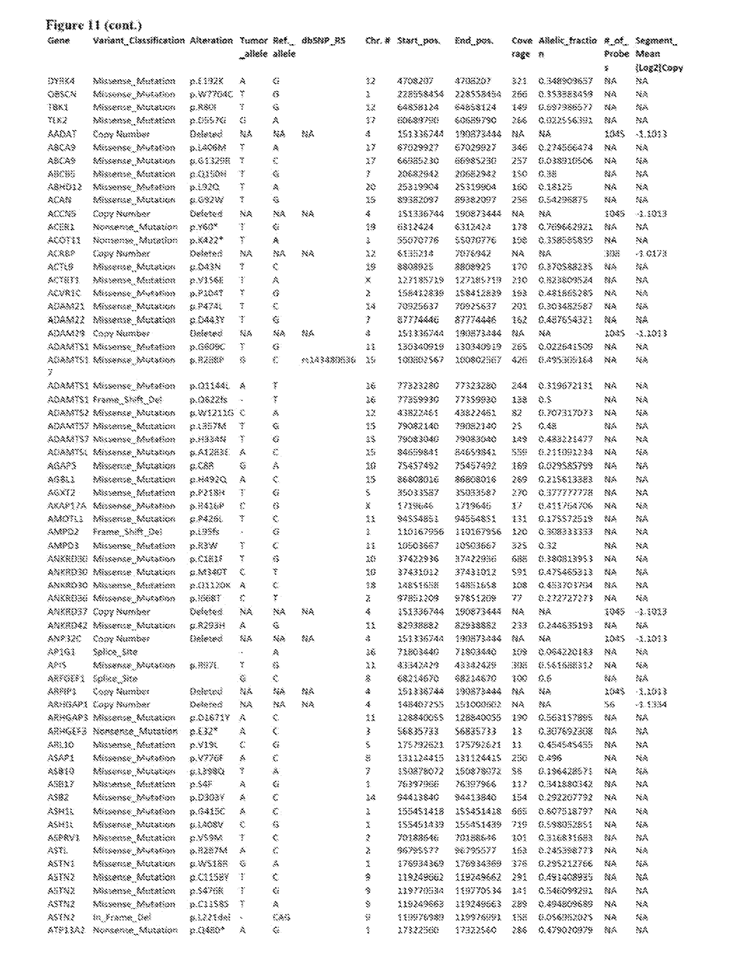

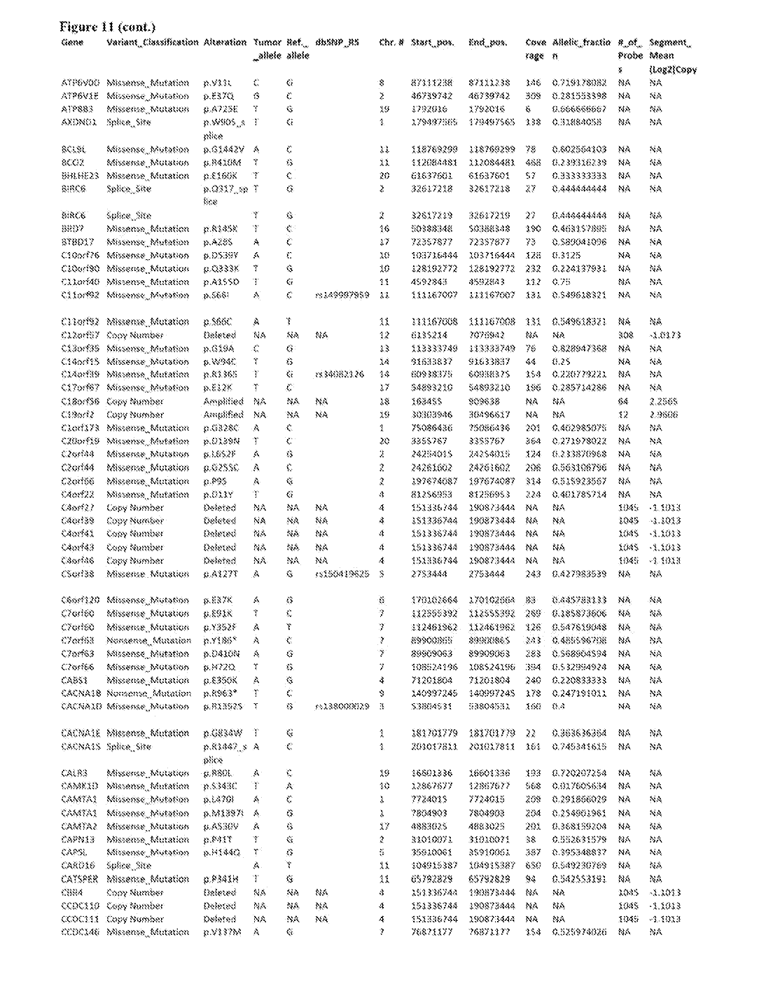

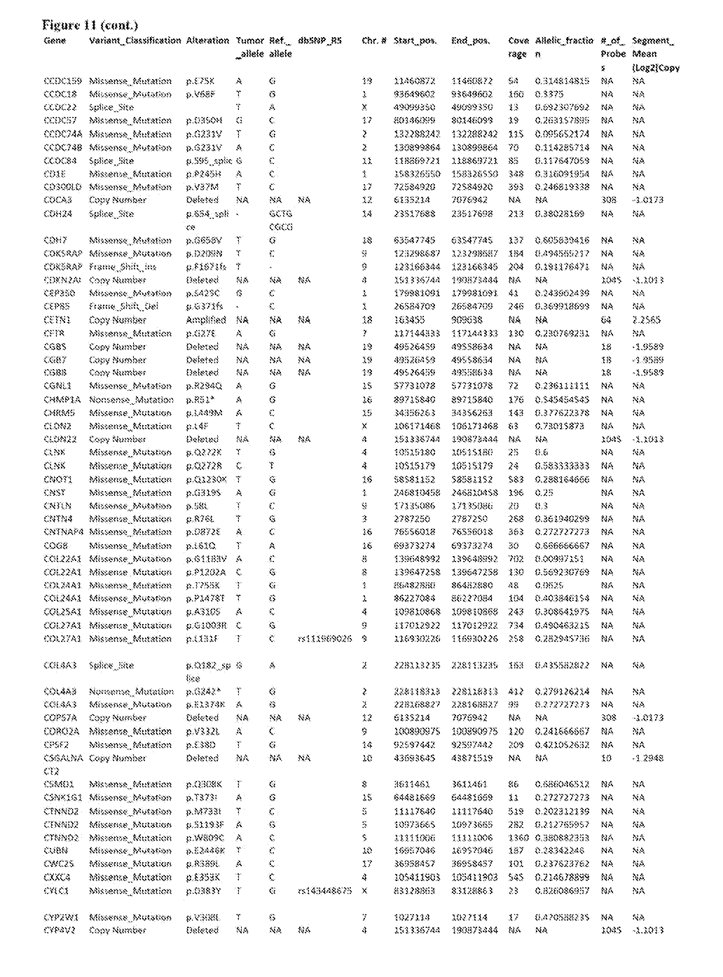

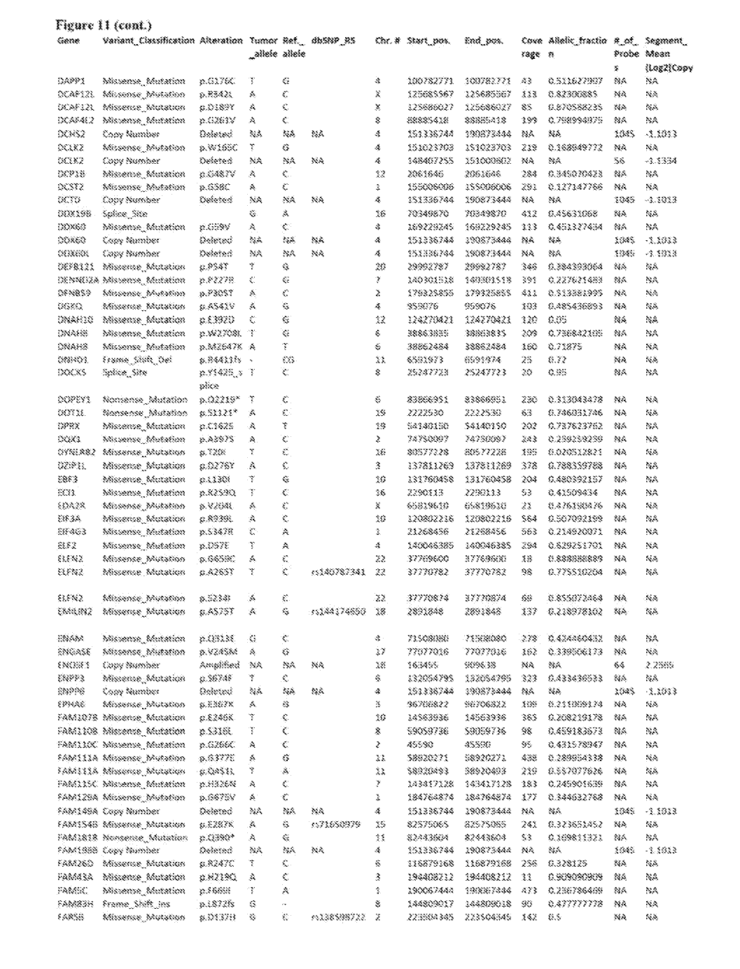

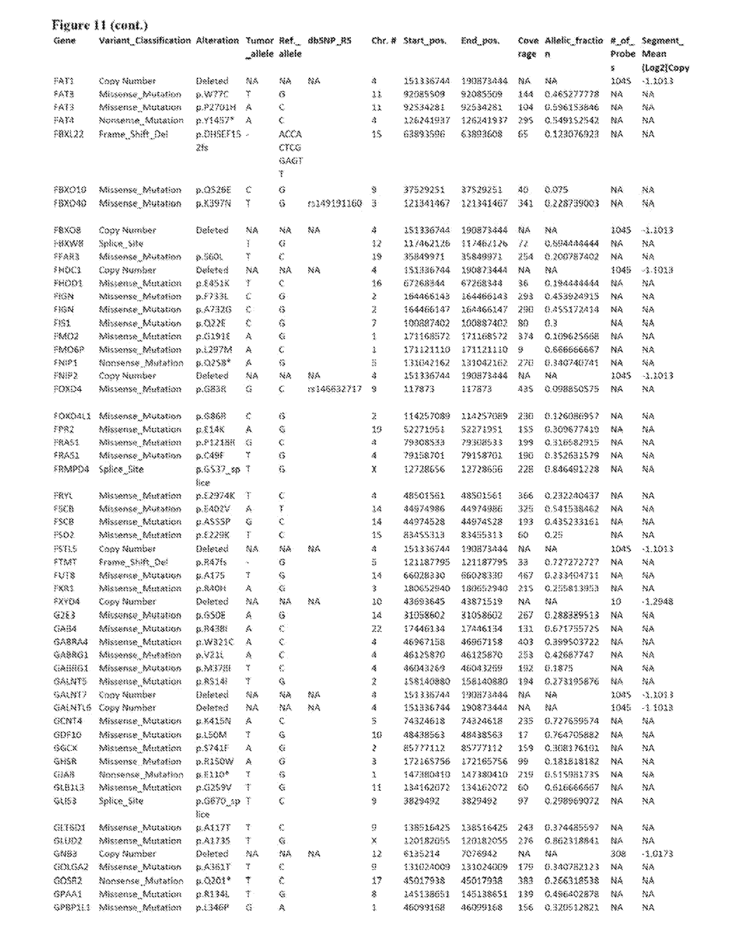

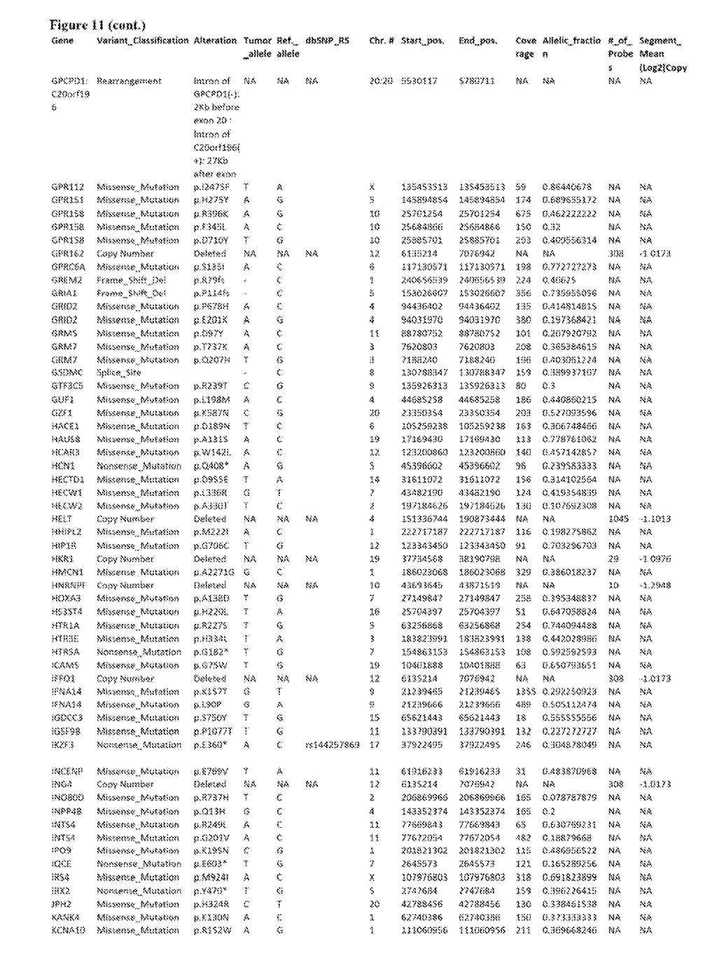

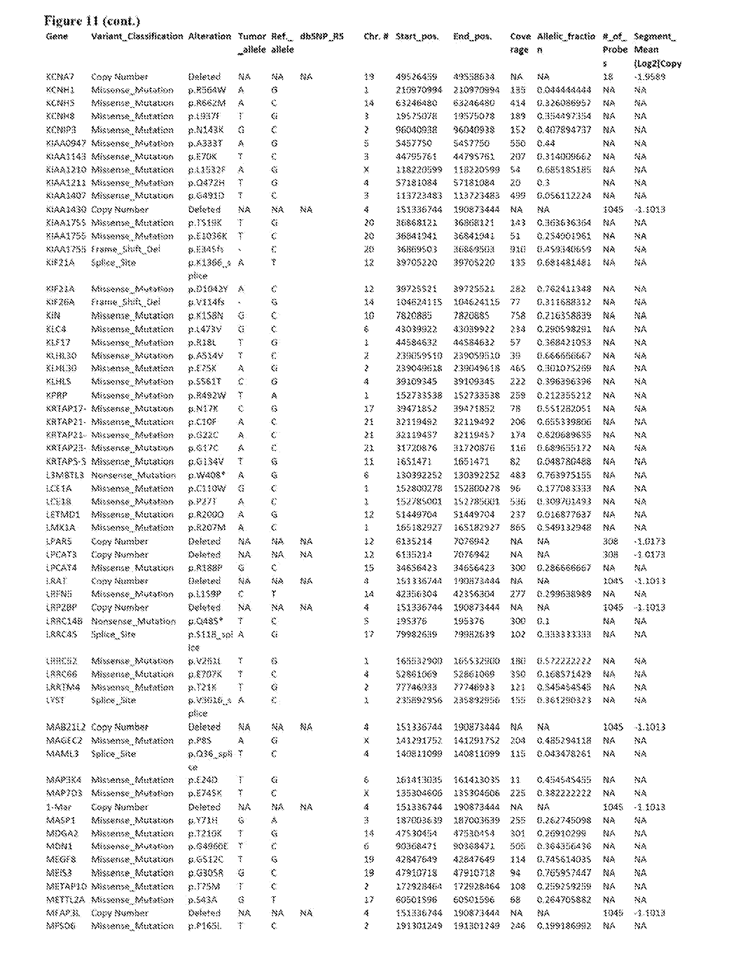

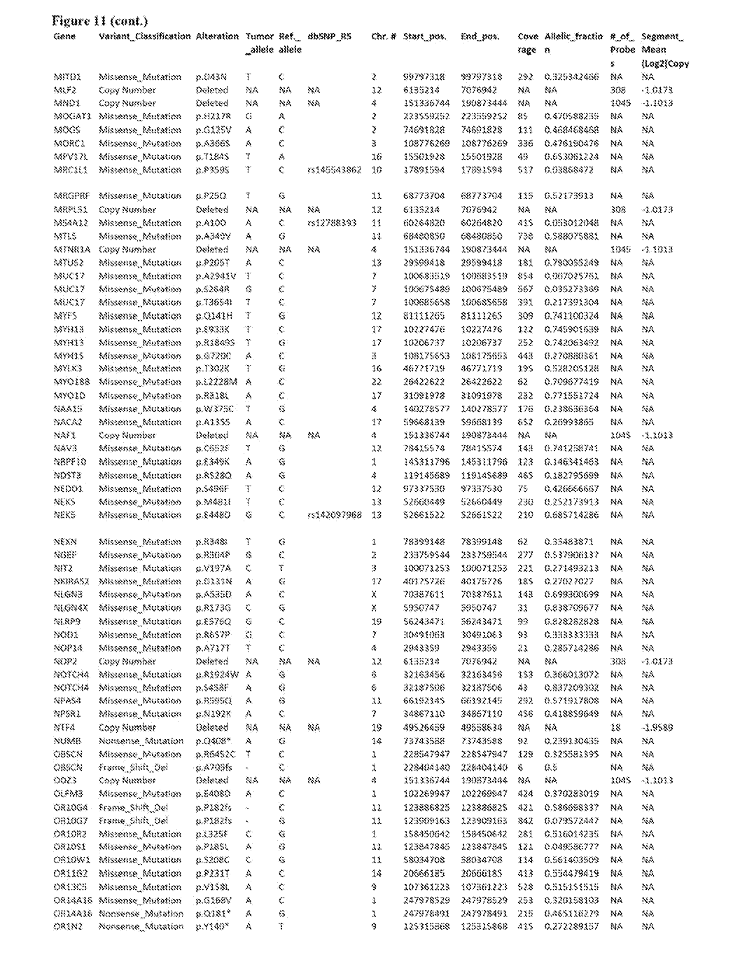

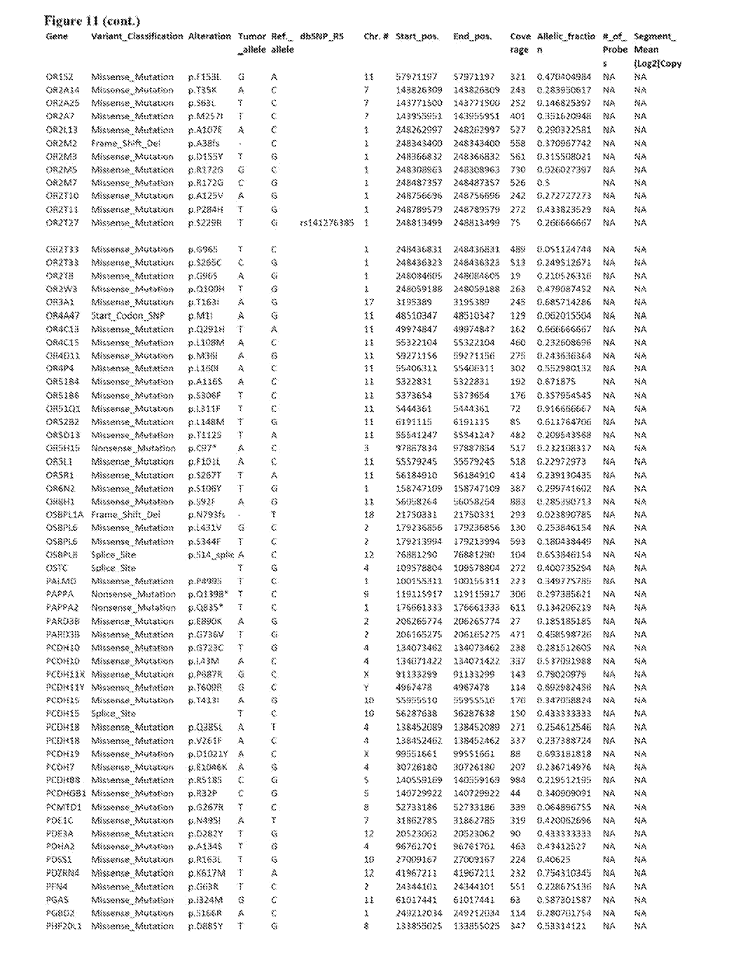

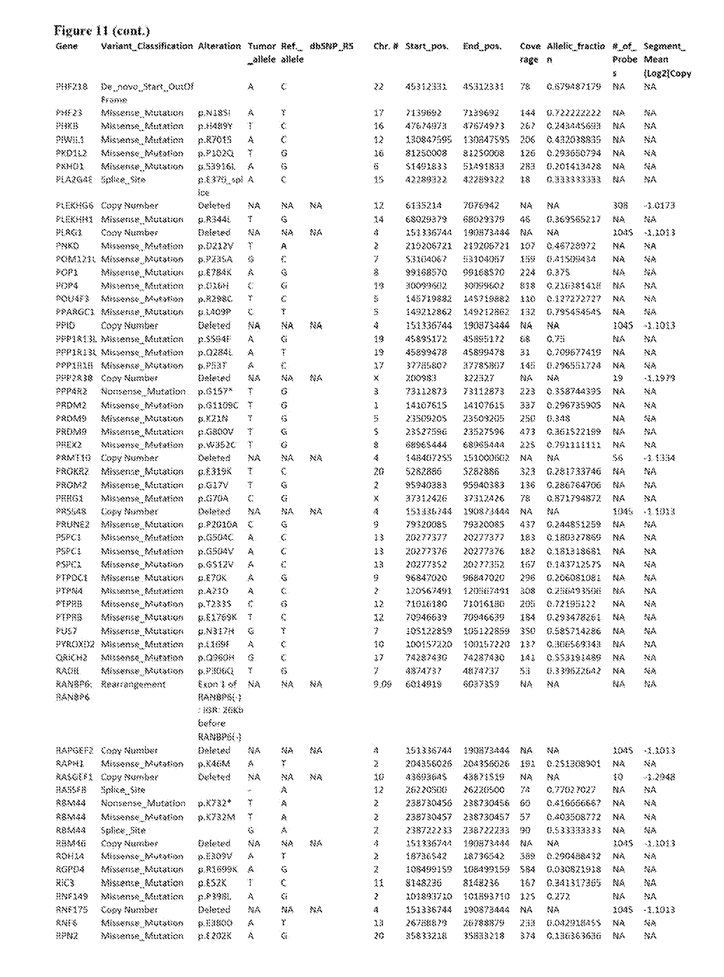

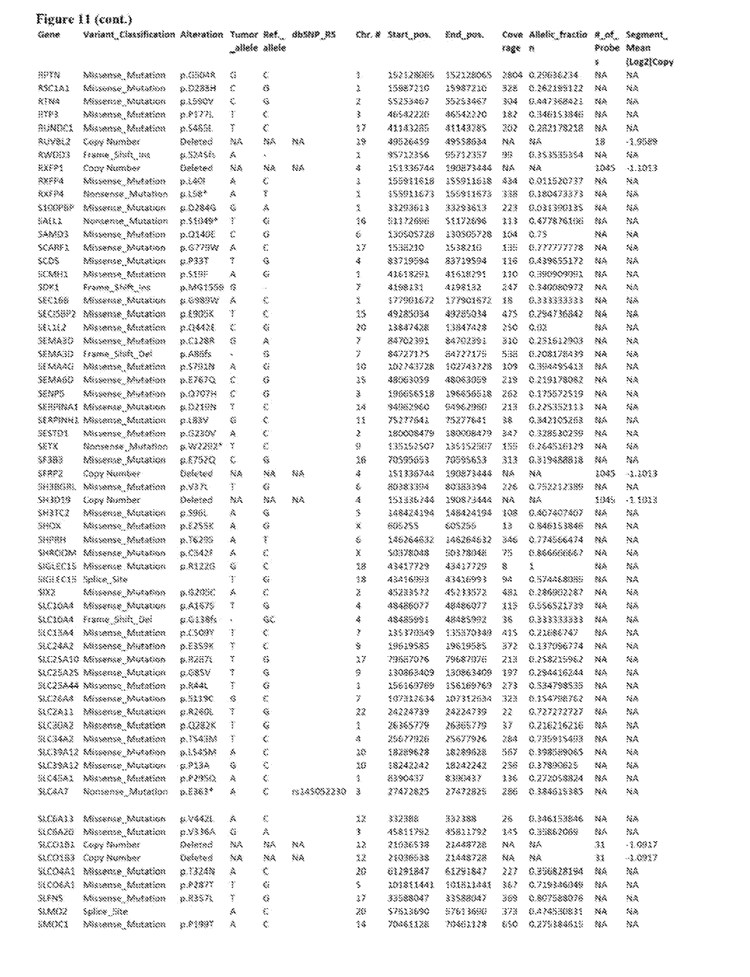

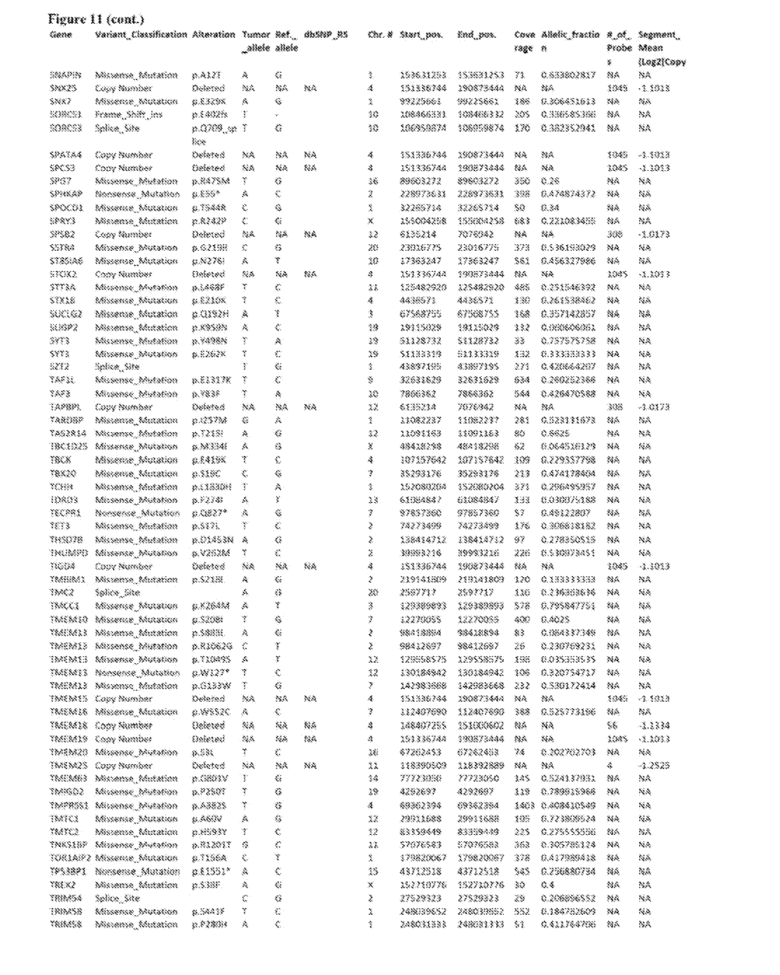

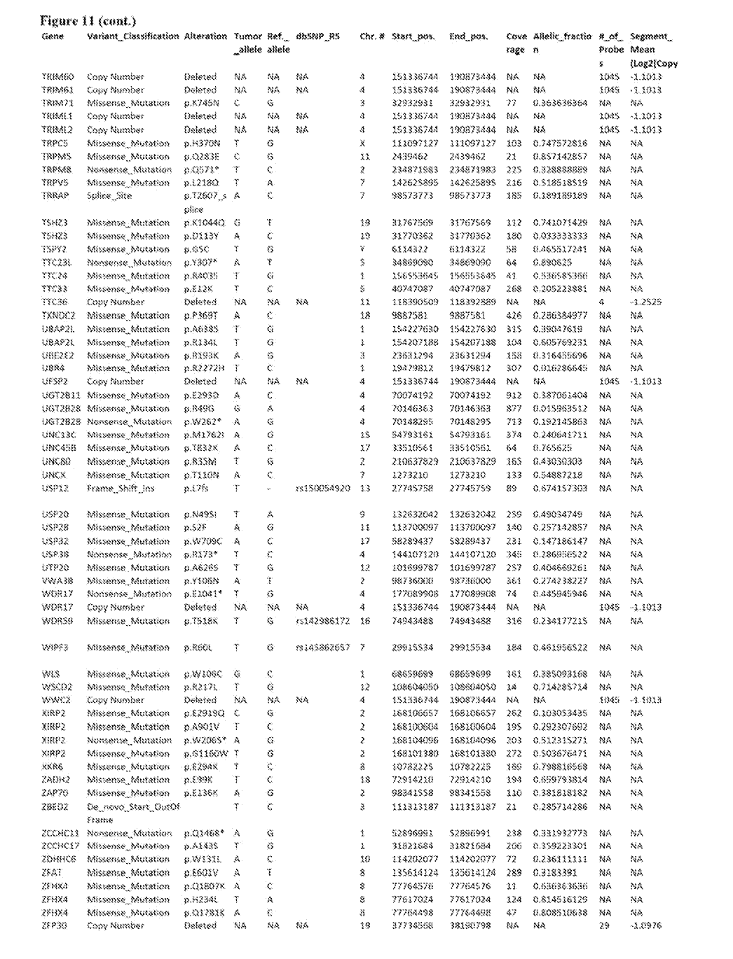

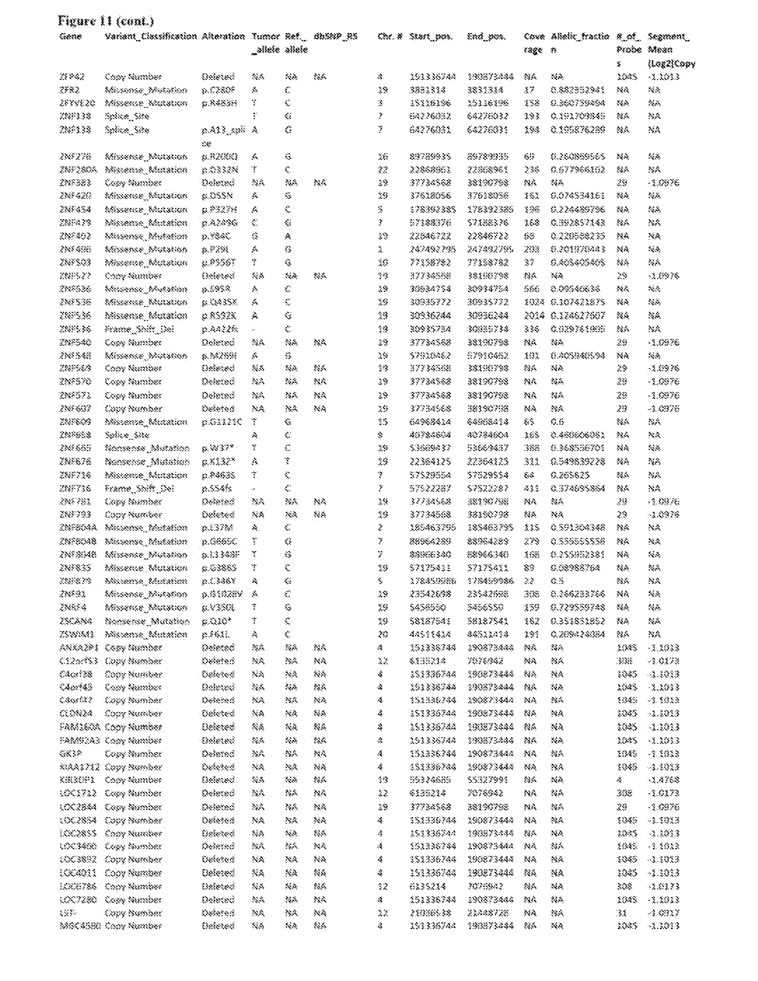

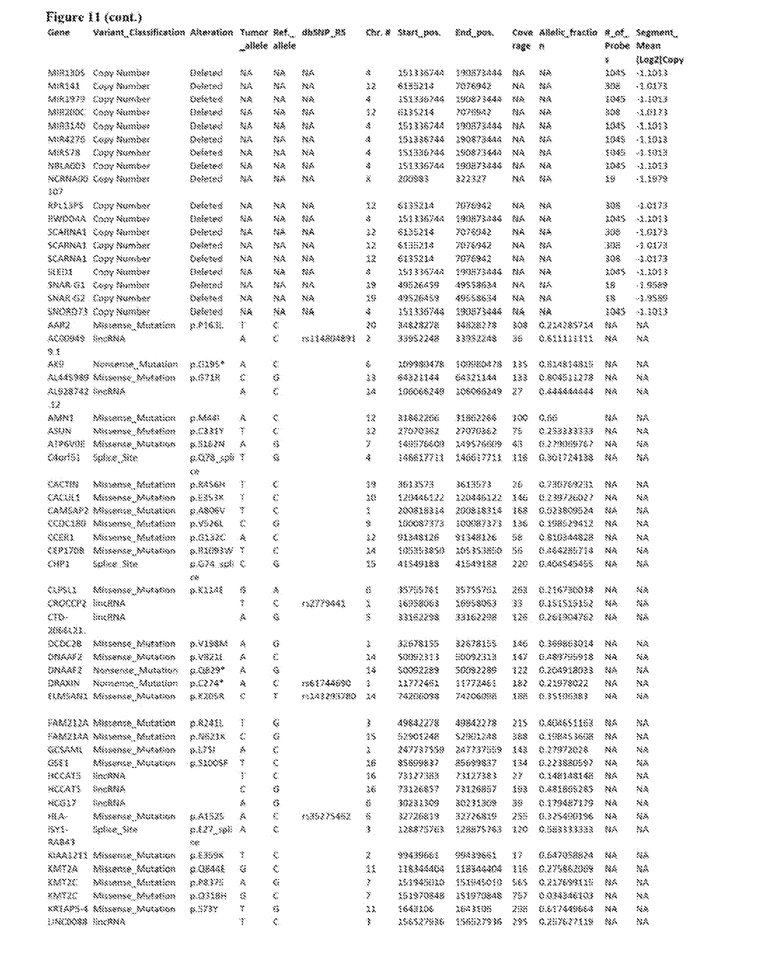

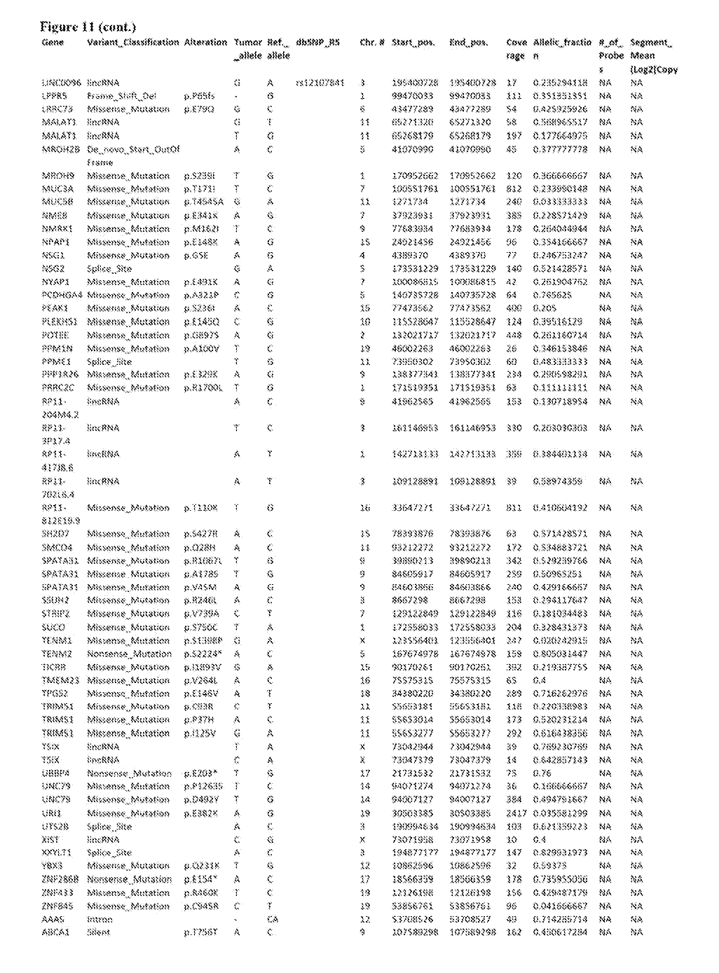

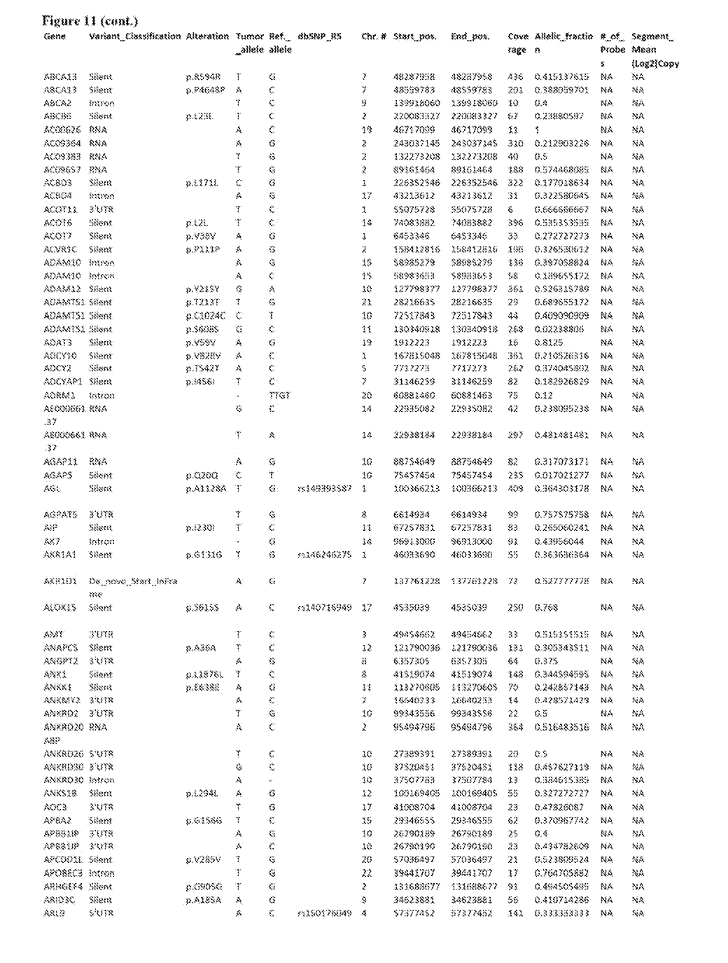

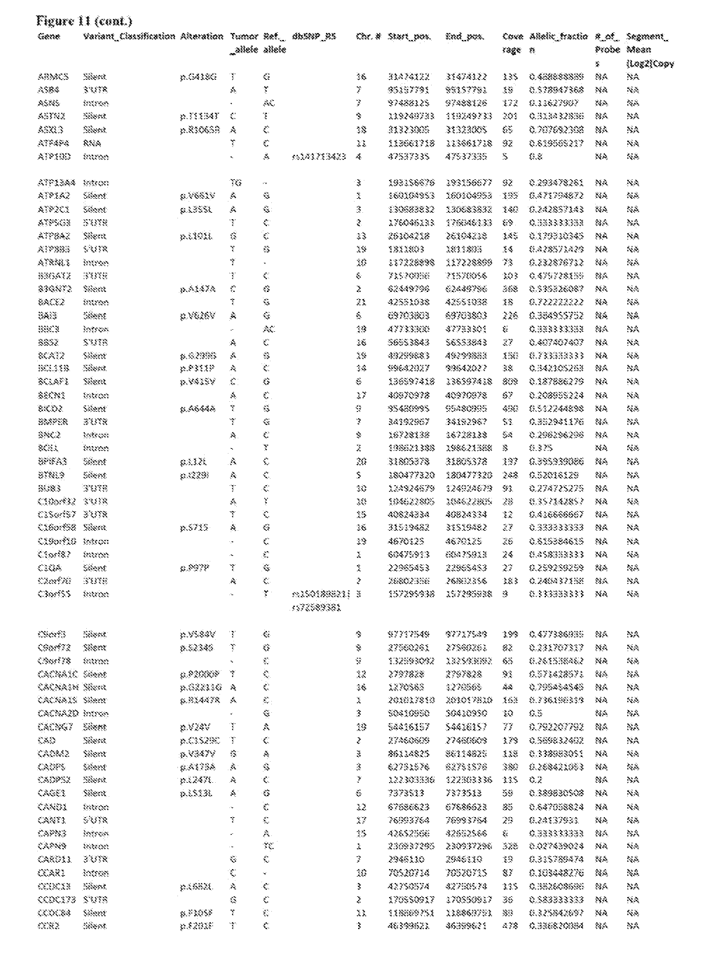

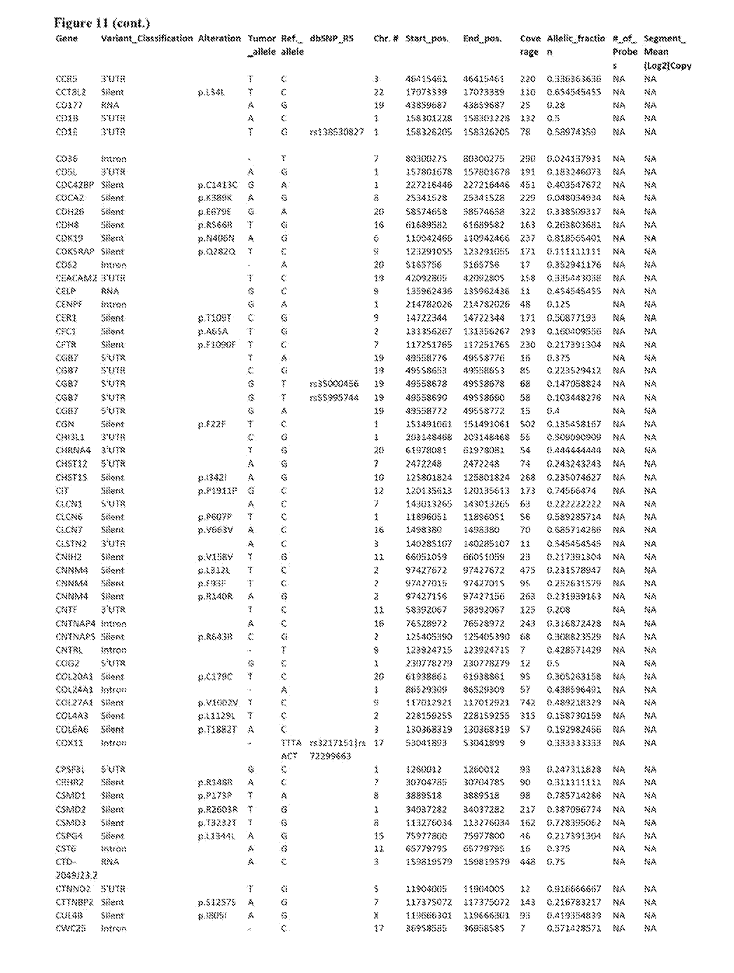

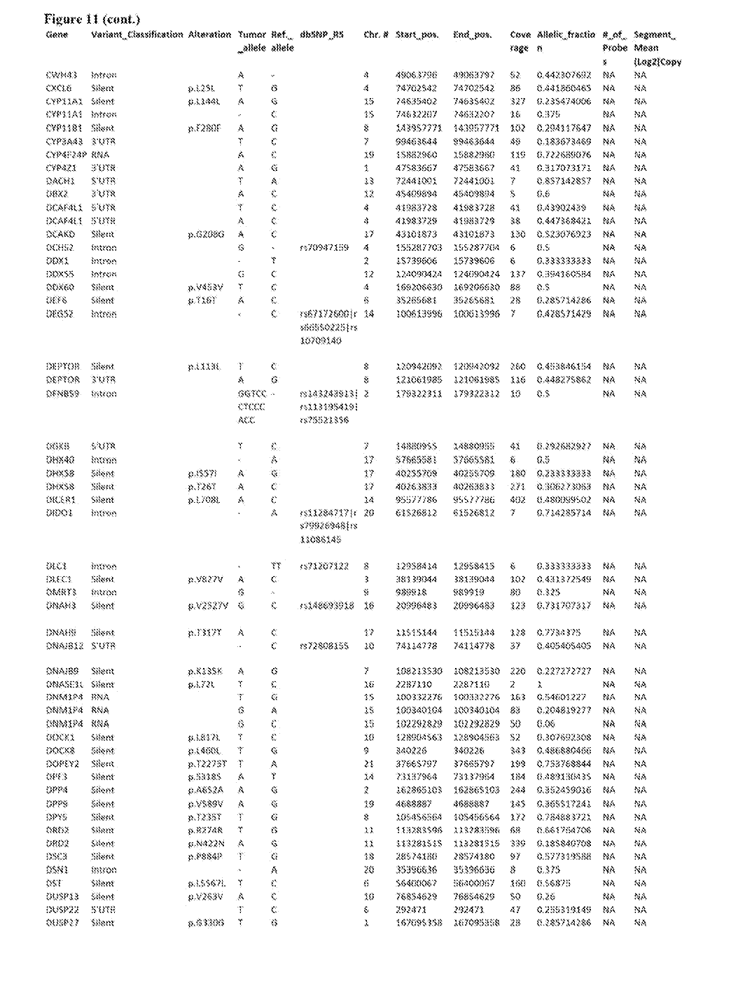

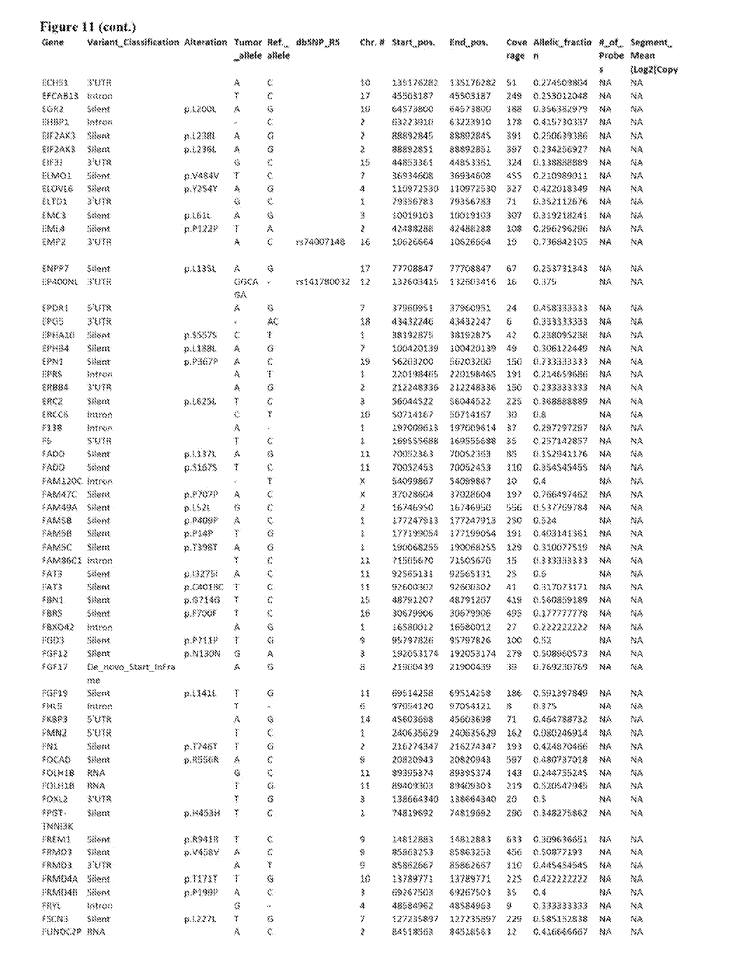

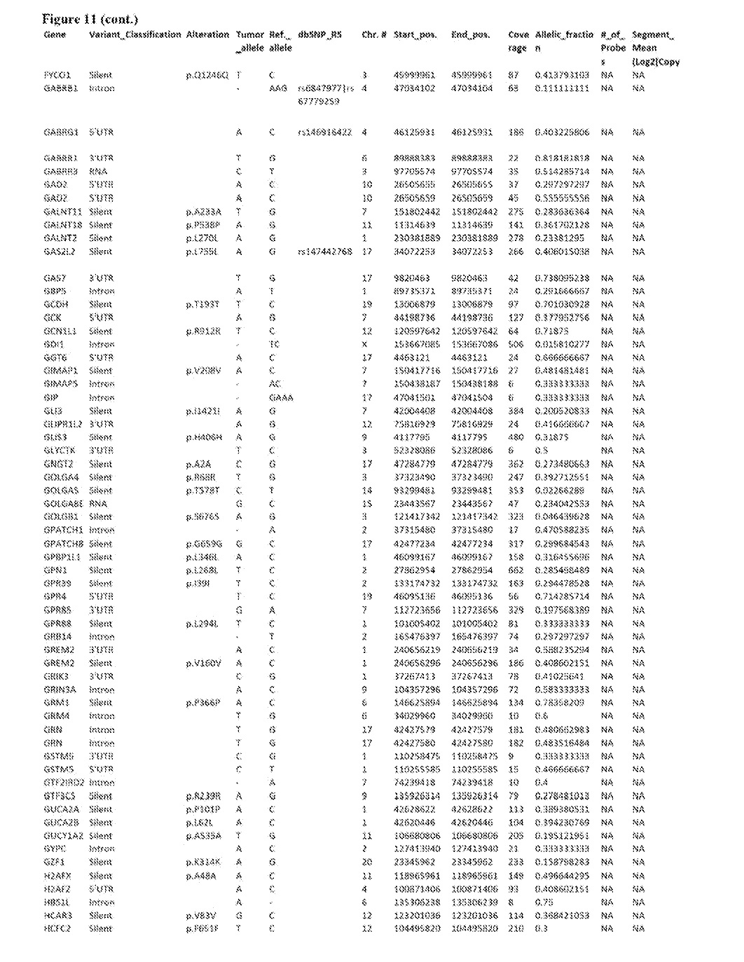

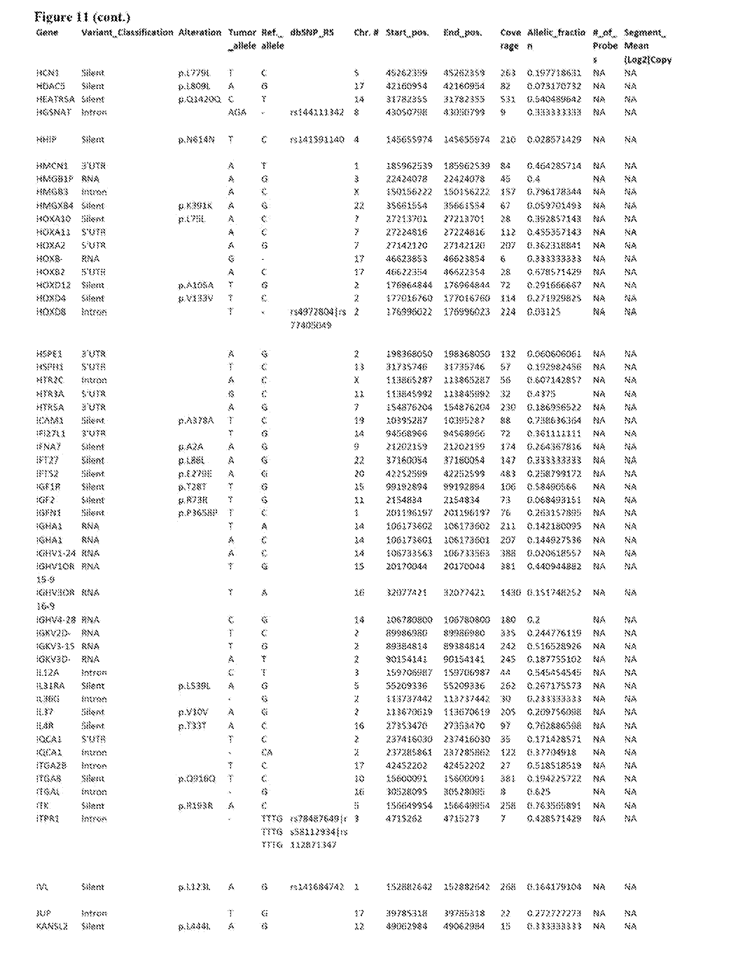

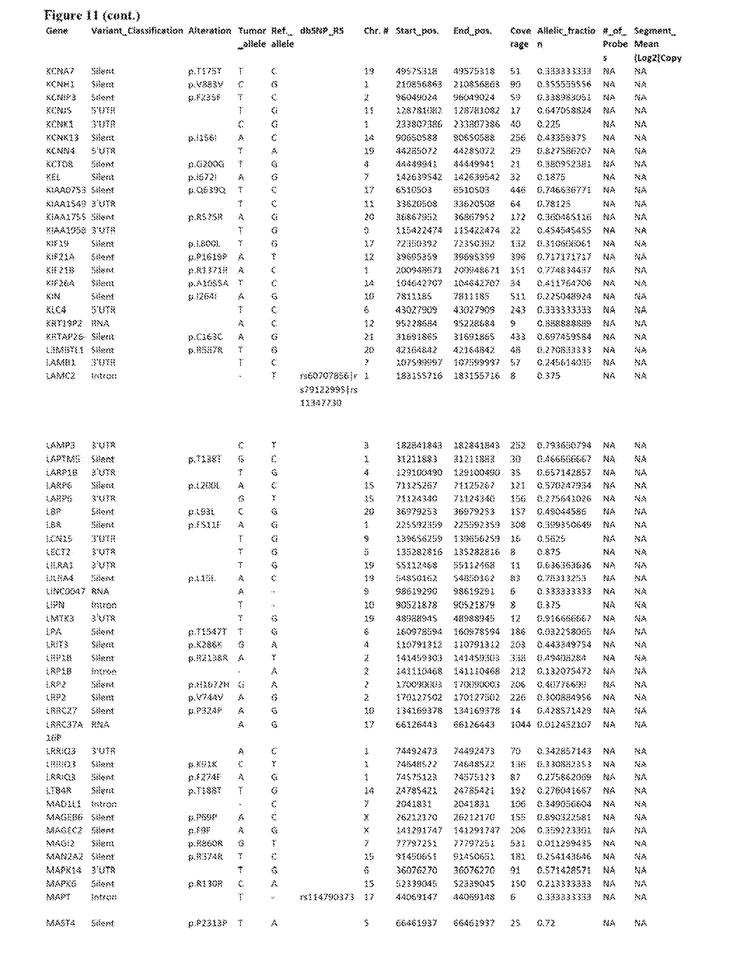

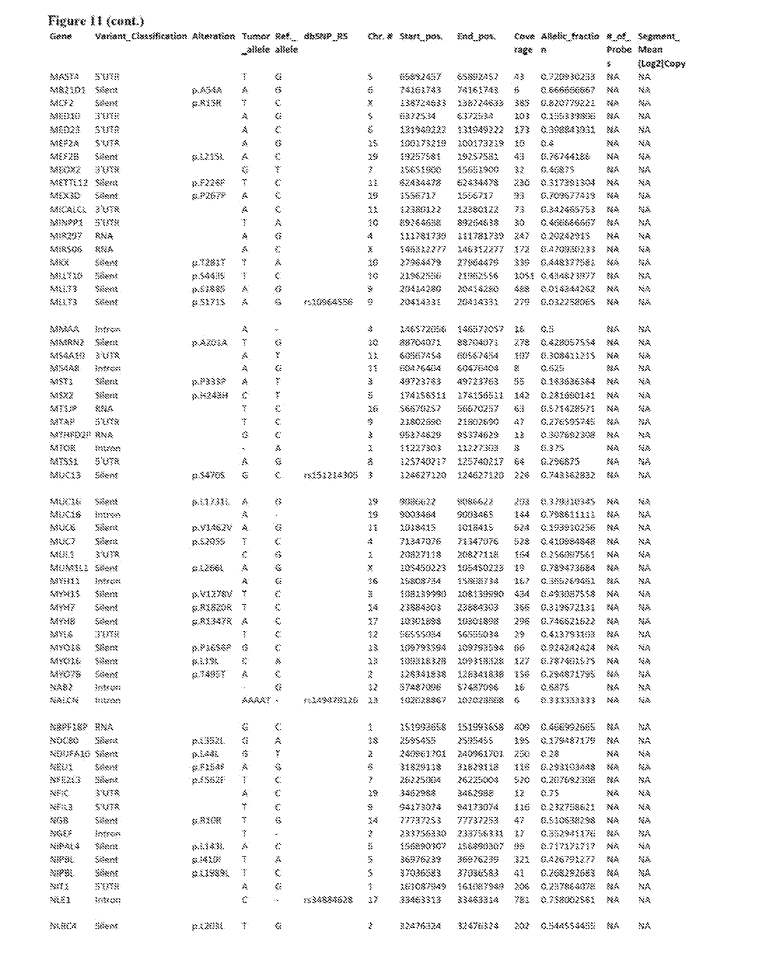

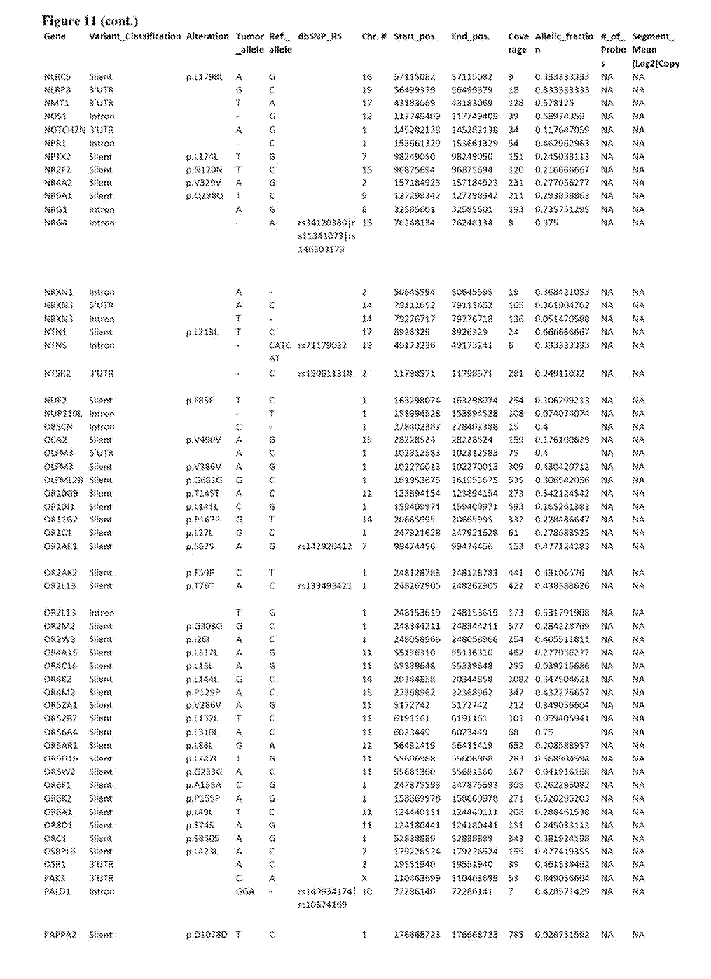

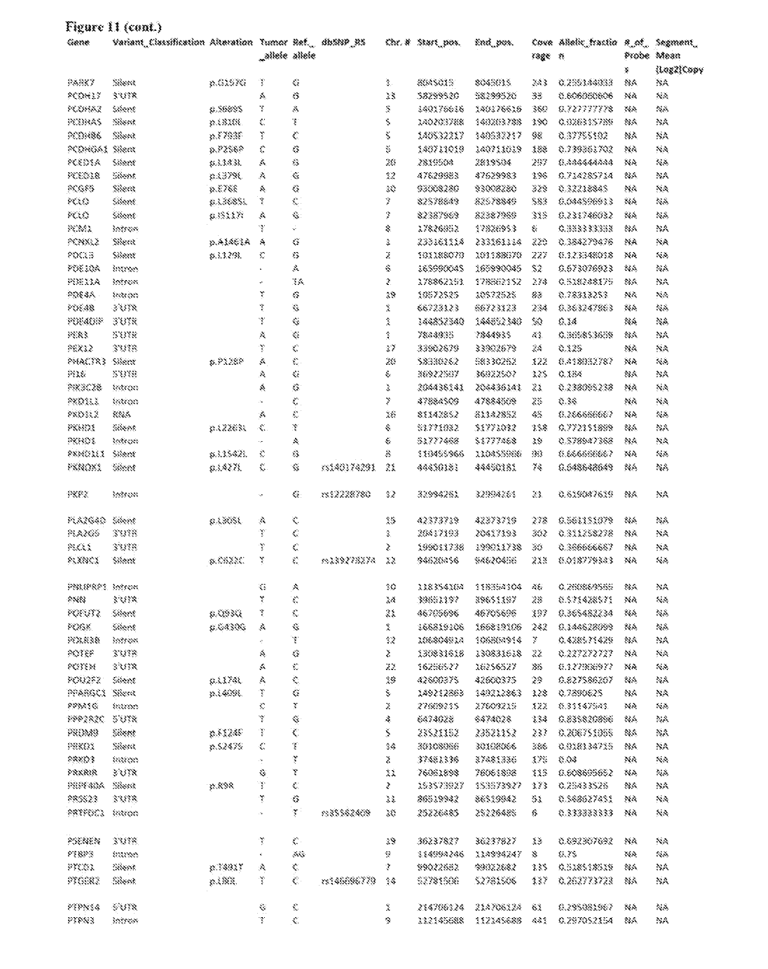

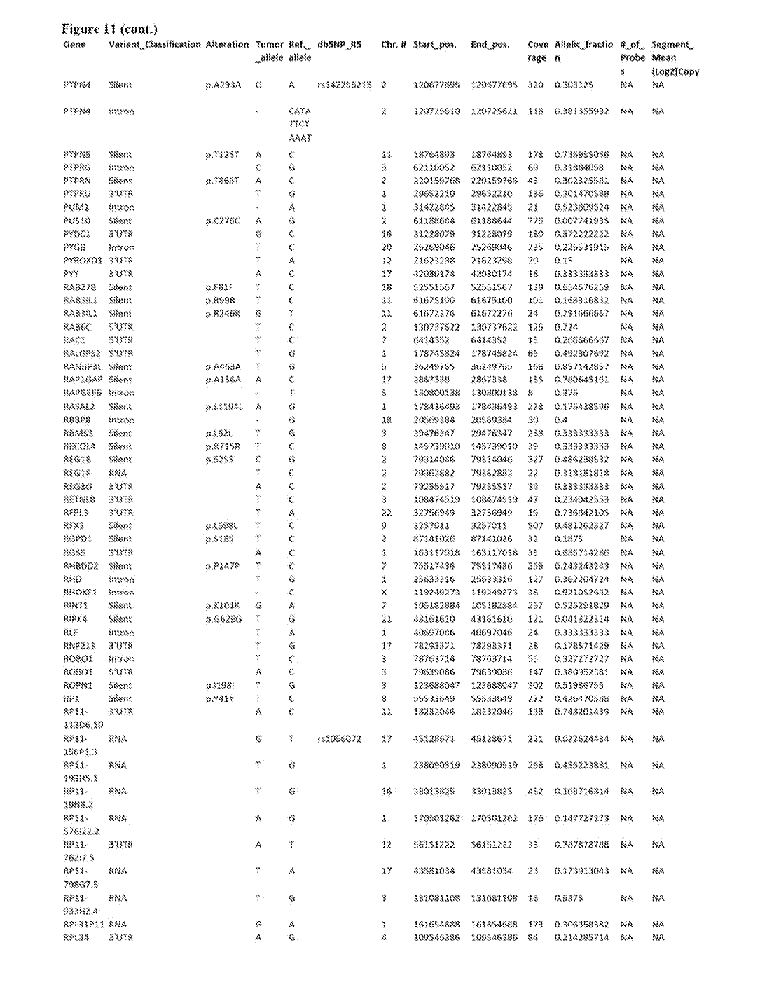

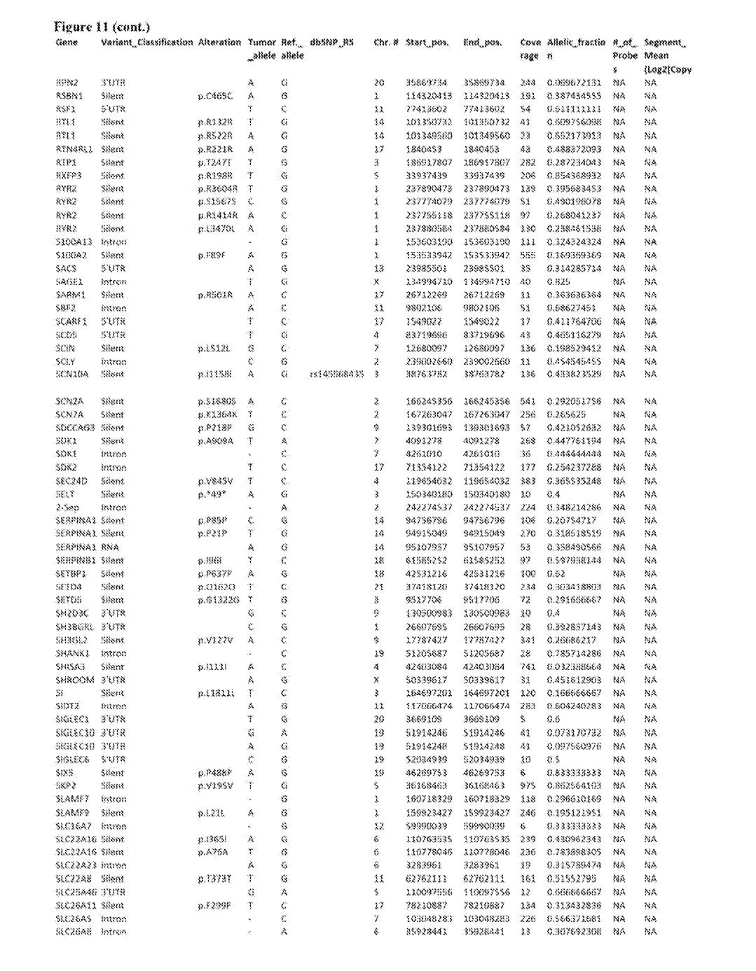

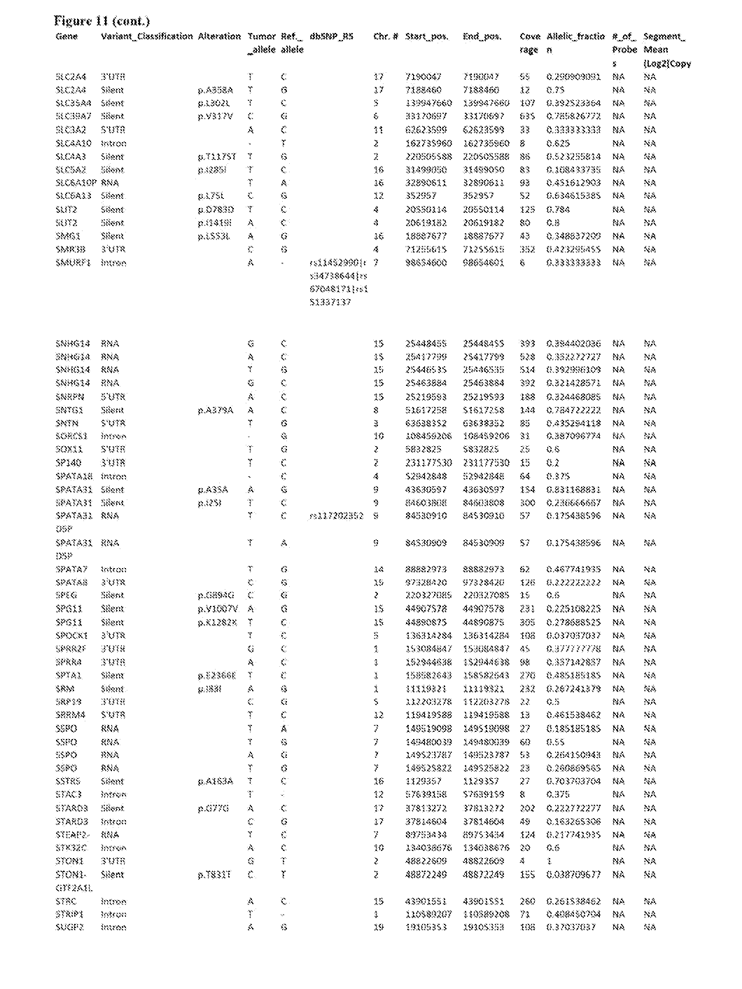

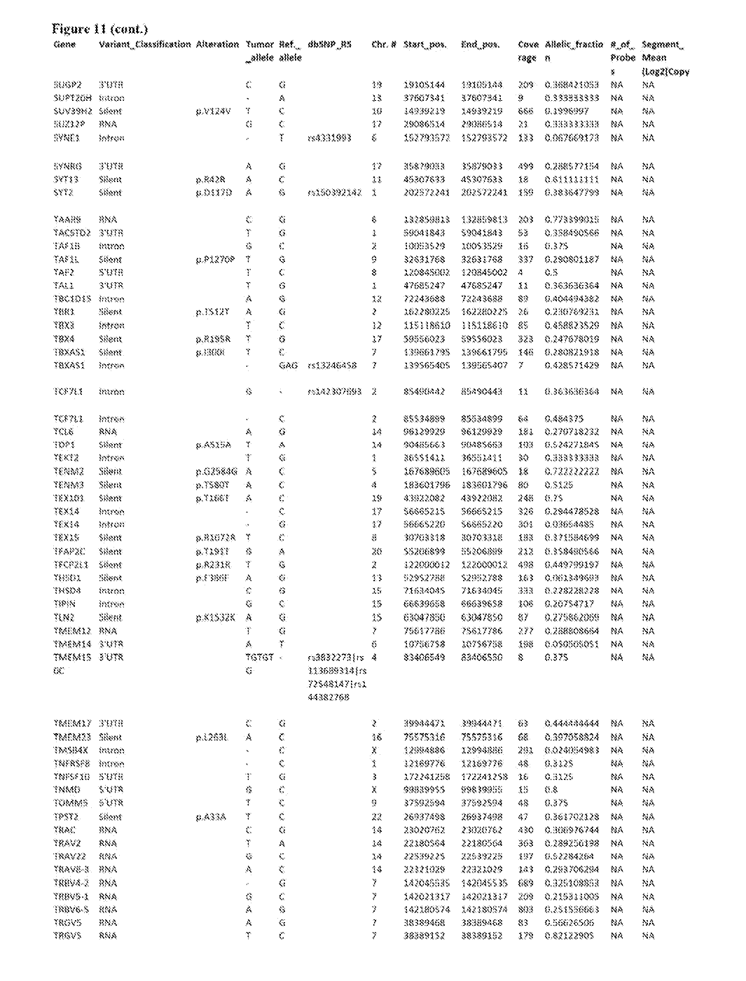

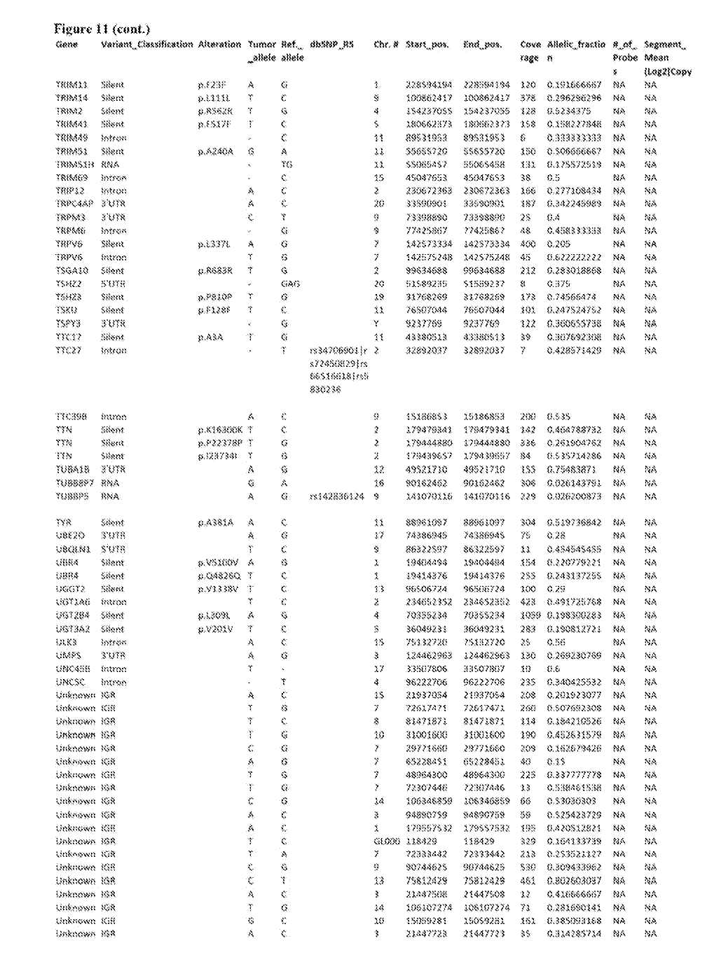

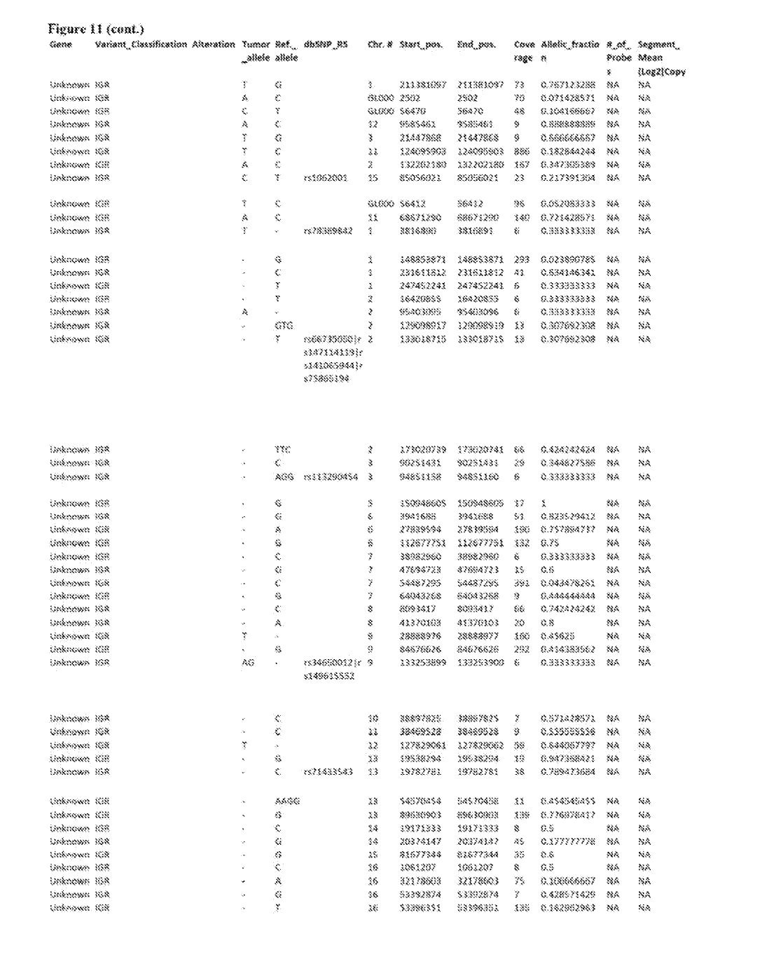

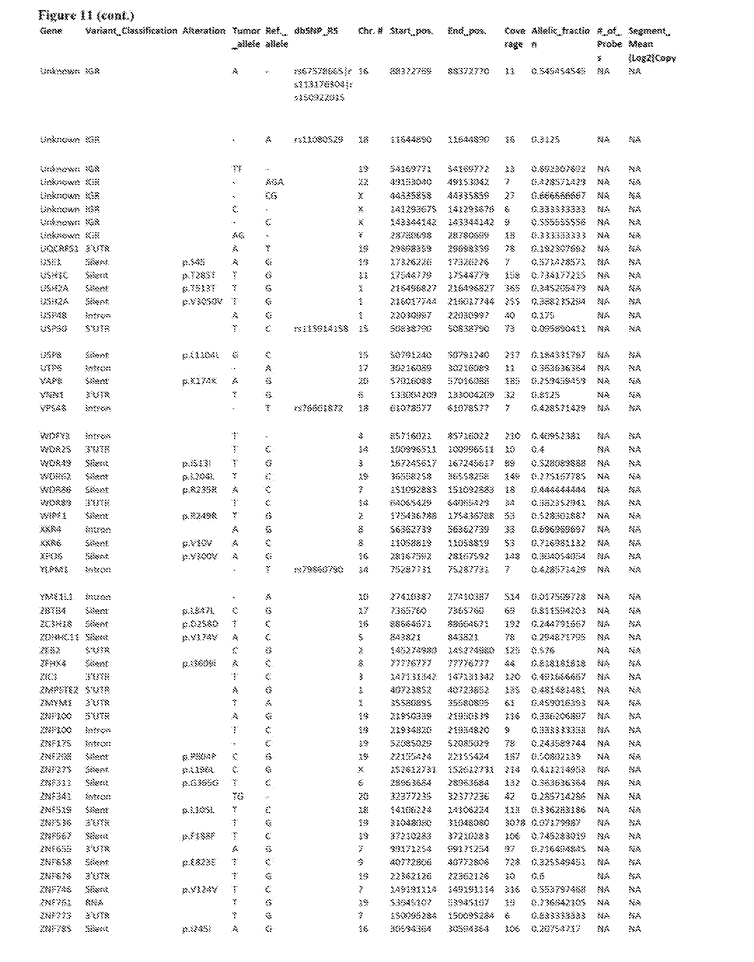

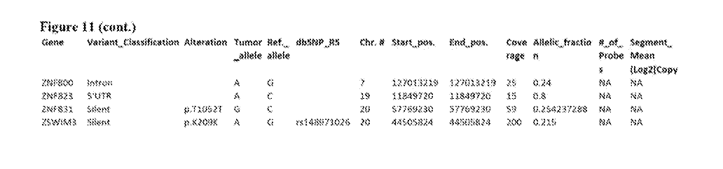

Next, the 1,767 non-synonymous somatic alterations observed in the WES data were ranked for clinical and biological relevance (FIGS. 6 and 11) (Van Allen et al. (2014) Nat. Med. 20:682-688). Among the clinically relevant events, a second somatic JAK3 missense mutation at codon 61 (S->C) was observed and orthogonally validated in tumor DNA with PCR (FIGS. 2A and 3B). This mutation occurred in the FERM domain and has not been described previously. Since two distinct genomic events were identified in JAK3 undergoing tumor somatic selection in cis, a scenario described in hematologic malignancies (Bergmann et al. (2014) Genes Chrom. Cancer 53:309-316), and Epstein-Barr Virus (EBV) induces PD-L1 expression via JAK3 (Green et al. (2012) Clin. Cancer Res. 18:1611-1618), whether these JAK3 mutations were activating and might contribute to PD-L1 mediated immune checkpoint evasion in lung cancer was determined.

### Example 3: Molecular Basis of JAK3 Auto-Activation and PD-L1 Overexpression

Constructs that express JAK3^{WT}, JAK3^{S61C}, JAK3^{V722I}, and JAK3^{S61C/V722I} were generated, and their activity was compared with an additional known activating JH2-domain mutation (JAK3^{R657Q}) (Zhu et al. (2014) Cancer Disc. 4:452-465), identified as a somatic mutation in squamous cell lung cancer (Cancer Genome Atlas Res. Network (2012) Nature 489:519-525). Consistent with the known impact of JH2 domain mutation on relieving JAK3 autoinhibition (Walters et al. (2006) Cancer Cell 10:65-75), transfection of 293T cells revealed that JAK3^{V722I}, JAK3^{S61C/V722I} or JAK3^{R657Q} overexpression resulted in increased JAK3 autophosphorylation and autoactivation compared with the wild-type control as measured by immunoblotting (FIG. 2D). Although levels of JAK3^{S61C} phosphorylation did not differ significantly from JAK3^{WT}, expression of JAK3^{S61C/V722I} caused the highest levels of JAK3 phosphorylation among all mutants, consistent with the positive selection observed in the tumor and cooperative gain of function.

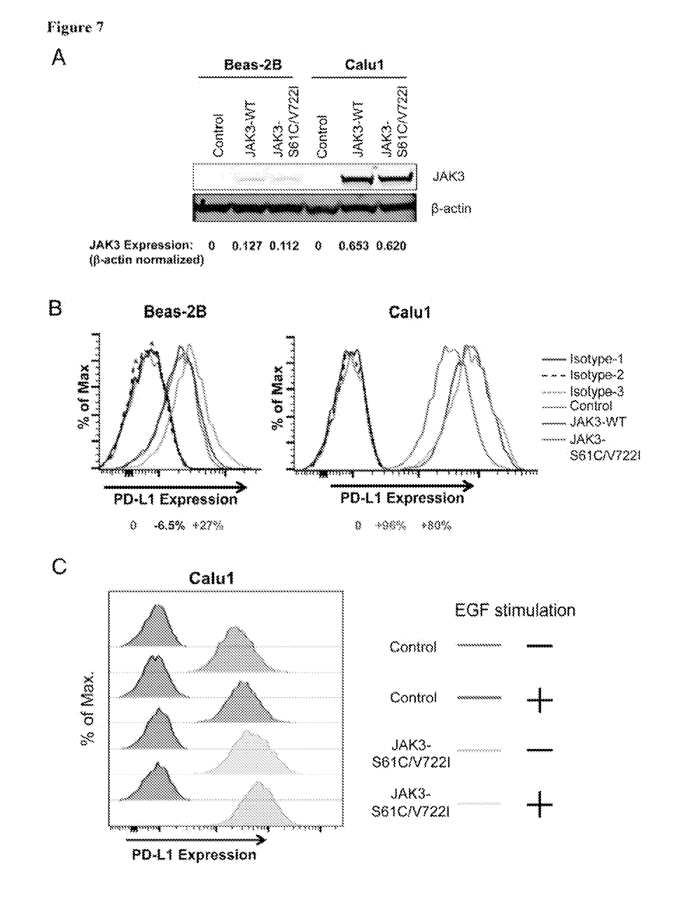

The consequences of stable JAK3 transduction on PD-L1 cell surface expression in immortalized lung epithelial cells (BEAS-2B) and lung cancer cells (Calu-1) was also determined (FIG. 7A). Low-level JAK3^{S61C/V722I} expression in BEAS-2B cells modestly induced surface PD-L1 by flow cytometry relative to control, as compared to no induction at all in JAK3^{WT} expressing cells (FIG. 7B). In contrast, 5-fold greater expression of JAK3 in Calu-1 increased PD-L1 levels more demonstrably, irrespective of the allele (FIG. 7).

The consequence of exposure to factors in the lung tumor microenvironment, such as EGF, was also determined since activation of EGFR signaling known to enhance PD-L1 expression in lung cancer (Azuma et al. (2014) Annal. Oncol. 25:1935-1940; Akbay et al. (2013) Cancer Disc. 3:1355-1363). Levels of PD-L1 in Calu-1 cells expressing JAK3^{S61C/V722I} were as high as control cells stimulated with EGF, and an additive further increase of PD-L1 expression in mutant cells upon EGF exposure was observed (FIG. 7C). These findings reveal that JAK3 activation in lung airway and cancer cells induces PD-L1, including wild type kinase, when overexpressed at high levels. Furthermore, activated JAK3 cooperates with factors such as EGF to boost PD-L1 expression even further.

### Example 4: Dual Impact on the Tumor and Immune Microenvironment

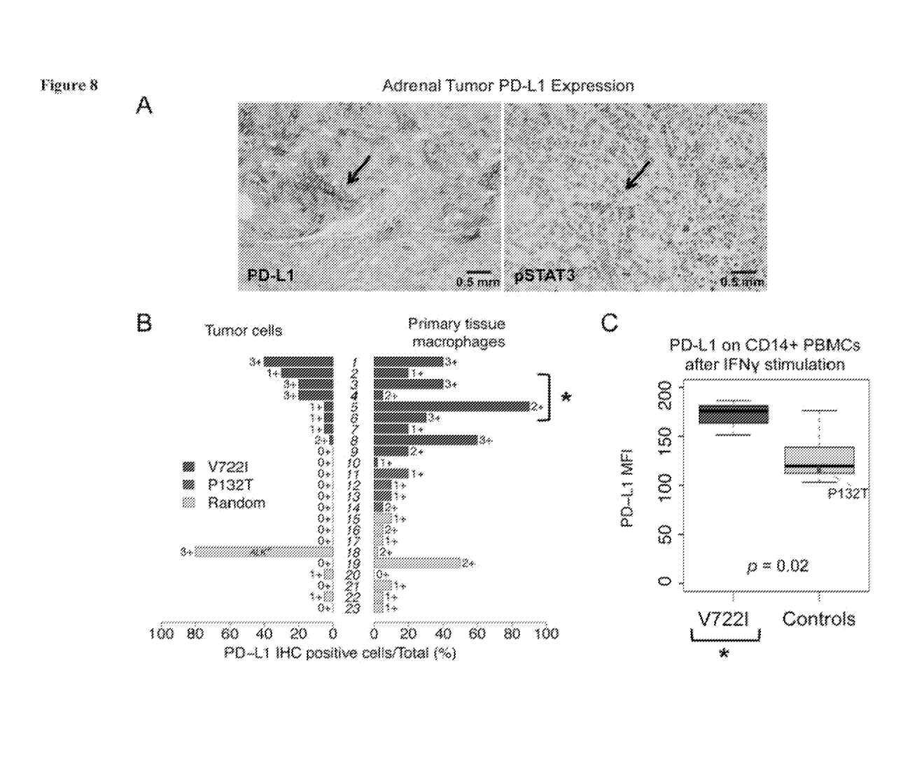

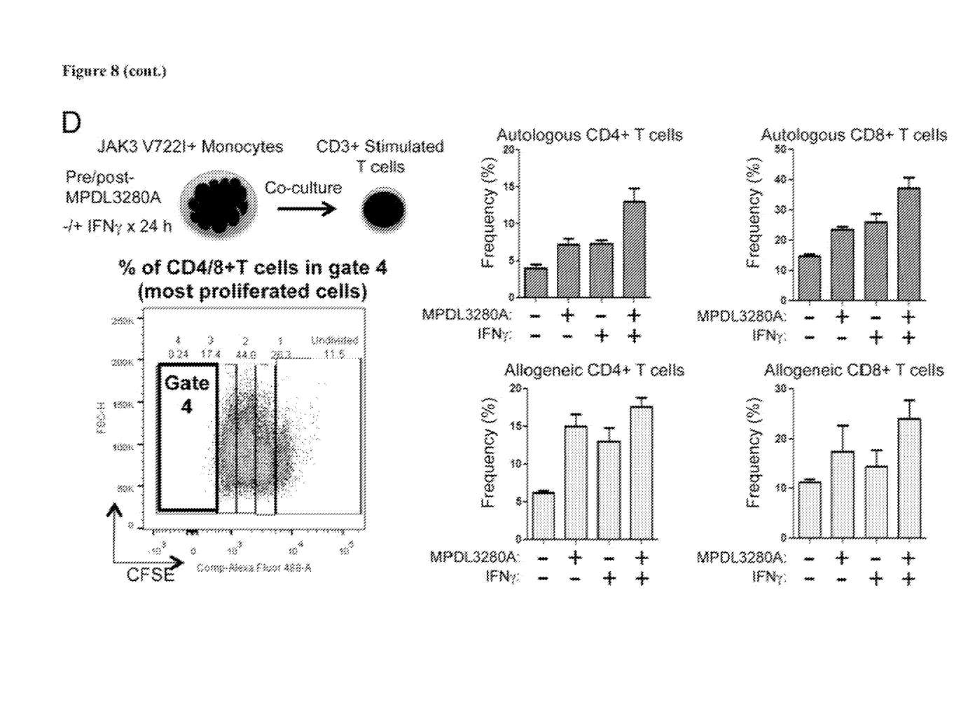

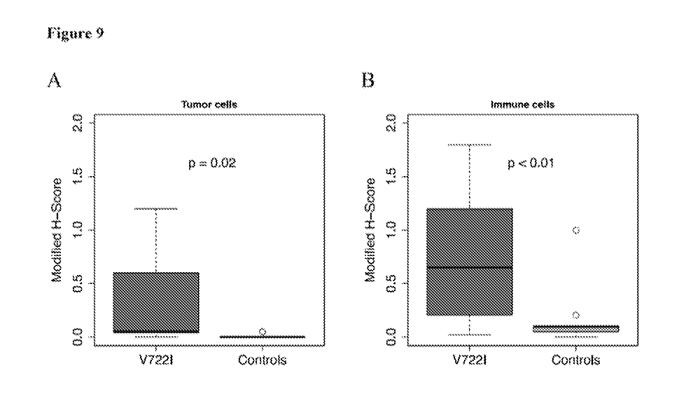

Consistent with these results, IHC of the patient's tumor using a validated antibody (Chen et al. (2013) Clin. Cancer Res. 19:3462-3473) revealed strong positive membrane PD-L1 expression on both tumor and immune cells (macrophages), coupled with increased nuclear pSTAT3 staining, a marker of JAK pathway activation (FIG. 8A). To assess the generalizability of this relationship, 10 out of 500 (lung adenocarcinoma patients (2%) previously genotyped for the JAK3^{V722I} mutation (MacConaill et al. (2014), J. Mol. Diagn. 16:660-672) at the institution were identified, including this index case. PD-L1 positivity was observed in tumor cells and more strikingly in macrophages in 9/10JAK3^{V722I} mutated cases (FIG. 8B). PD-L1 positivity was substantially enriched as compared to a random control set of lung cancers (tumor cells: p=0.02; immune cells: p<0.01; Mann-Whitney) (FIG. 9), including 4 patients carrying the JAK3^{P132T} variant, with the exception of high level PD-L1 tumor expression associated with an ALK rearrangement and another tumor with high level PD-L1 expression in macrophages (FIG. 8B).

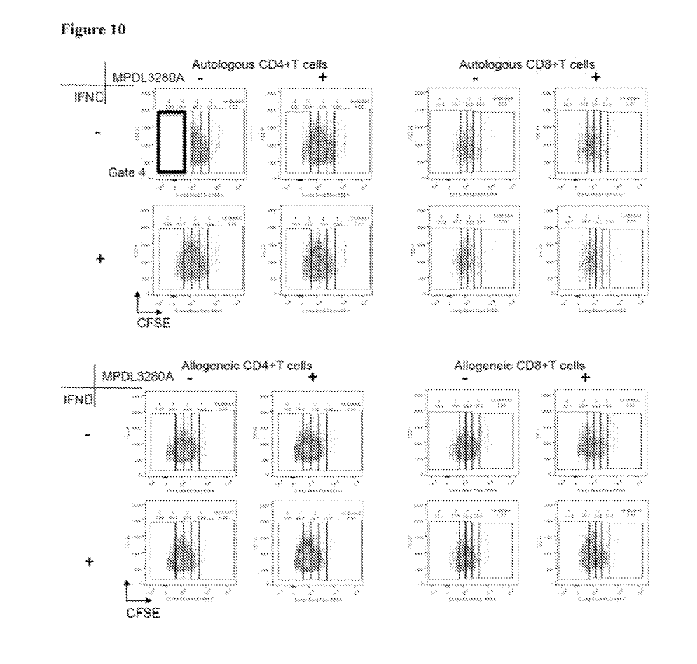

Because of the strong activation in the immune compartment, and the presence of JAK3^{V722I} in the germline, the inducibility of PD-L1 expression in available matched patient PBMCs was determined. Stimulation with IFN-γ, another cytokine known to trigger PD-L1 in the tumor immune microenvironment, resulted in modest but significantly increased expression of PD-L1 on CD14+ myeloid cells from JAK3^{V722I} positive patients compared to a JAK3^{P132T} positive patient or negative blood donor controls (FIG. 8C). Next, to determine if this increased PD-L1 expression directly inhibits T cells, blood from the index patient immediately pre- and 1 h post-MPDL3280A infusion was collected and monocytes from these samples were exposed to the patient's own activated T cells, or from allogeneic T cells from a different donor. In both instances, it was found that T cell activation was significantly greater in the presence of circulating MPDL3280A, especially when monocytes were primed with IFNγ (FIGS. 8D and 10). This enhanced T cell activity correlated with the clinical response that was observed upon MPDL3280A rechallenge (FIG. 1D). Thus, monocytes/macrophages that carry the JAK3^{V722I} allele also express increased levels of PD-L1, which can contribute directly to T-cell suppression.

Taken together, these findings indicate that, in addition to somatic alteration in lung cancer cells, germline expression of the JAK3^{V722I} allele in infiltrating immune cells represents a key contributor of PD-L1 tumor immune checkpoint engagement.

Immune targeting of the PD-L1/PD1 interaction is emerging as an effective therapy for multiple aggressive tumor types, including non-small cell lung cancer (Topalian et al. (2012) New Engl. J. Med. 366:2443-2454), and results in occasional long-term responses. While tumor or immune cell PD-L1 expression may indicate a suppressed immune microenvironment and enrich for clinical activity (Taube et al. (2014) Clin. Cancer Res. 20:5064-5074), the molecular basis and markers of response remain unclear.

A patient with metastatic lung adenocarcinoma who experienced an exceptional and durable response to PD-L1 inhibition was genomically characterized. One germline JAK3 variant and one somatic JAK3 mutation was determined in the patient's tumor in cis, and it was demonstrated that these genetic alterations act in concert to activate JAK3. Stable transduction of this double-mutant increased PD-L1 expression in lung cells. Furthermore, the presence of JAK3^{V722I} in the germline, the strong tumor immune cell PD-L1 positivity, and the enhanced PD-L1 induction by IFN-γ in monocytes, which inhibits T cell activation in an MPDL3280A sensitive manner, also indicate a more complex interaction with the tumor microenvironment. It is believed that this is the first report that illustrates how a genomic mechanism that impacts both tumor cells and the host response enhances PD-L1 expression and immune evasion by engaging the PD1 immune checkpoint.

Multiple reports have identified candidate mechanisms that may predict response to immune checkpoint blockade. These may include high levels of tumor-specific neoantigens (Brown et al. (2014) (Genome Res. 24:743-750: Snyder et al. (2014) New Eng. J. Med. 371:2189-2199) or inherited immune-related characteristics (Breunis et al. (2008) J. Immunother. 31:586-590). Notably, JAK3 signaling regulates EBV mediated PD-L1 expression in lymphomas (Green et al. (2012) Clin. Cancer Res. 18:1611-1618) and has been implicated in response to PD-1 blockade in Hodgkin's lymphoma (Ansell et al. (2015) New Engl. J. Med. 372:311-319), STAT3 binds directly to the PD-L1 promoter (Wolfle et al. (2011) Eur. J. Immunol. 41:413-424), and other activating mutations, such as JAK3^{R657Q}, are found in lung cancer (Cancer Genome Atlas Res. Network (2012) Nature 489:519-525), supporting the potential generalizability of the findings described herein.

In light of the determinations described herein that JAK3^{WT} overexpression also induced PD-L1, it is notable that the JAK2 amplicon, which also contains the PD-L1 locus, is recurrently amplified in lymphoma and EBV-positive gastric cancer (Green et al. (2010) Blood 116:3268-3277: Cancer Genome Atlas Res. Network (2014) Nature 513:202-209). Thus, functional SNP variants, somatic alterations resulting in activation of other JAK family members (e.g., JAK1, JAK2, or TYK2), or inactivation of negative regulators, such as suppressor of cytokine signaling (SOCS) family members may serve as a common pathway for upregulating PD-L expression and predicting responsiveness to this immune therapy. Indeed, since this particular JAK3^{V722I} variant is present in the germline at a significantly lower frequency (1-2%) compared with the frequency of PD-L1 positivity in lung cancer overall (at least 20%), it may only explain a small subset of tumors that engage this pathway. But since long-term durable remissions are much rarer, it is possible that studies in patients enriched for this genotype may show similarly impressive responsiveness as seen in this case.

The results described herein expand the concept of studying extraordinary responses to cancer therapeutics beyond classical targeted therapies to include approved or investigational immunotherapies. The identification of these and other genomic mechanisms of sensitivity to immune checkpoint inhibitor blockade will not only help tailor this therapy in a personalized fashion, but may also suggest pharmacologic approaches to induce sensitivity in otherwise resistant patients. Finally, profiling patients who demonstrate initial responses but develop acquired resistance may further illuminate the spectrum of pathways that restrict tumor immunity and implicate additional rational modalities for therapeutic development.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web and/or the National Center for Biotechnology Information (NCBI) on the world wide web.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of determining whether a subject afflicted with a cancer or at risk for developing a cancer would benefit from anti-immune checkpoint inhibitor therapy, the method comprising:
a) obtaining a biological sample from the subject;
b) determining the presence, copy number, amount, and/or activity of at least one biomarker listed in Table 1 in a subject sample;
c) determining the presence, copy number, amount, and/or activity of the at least one biomarker in a control; and
d) comparing the presence, copy number, amount, and/or activity of said at least one biomarker detected in steps b) and c);
wherein the presence or a significant increase in the copy number, amount, and/or activity of the at least one biomarker in the subject sample relative to the control indicates that the subject afflicted with the cancer or at risk for developing the cancer would benefit from anti-immune checkpoint inhibitor therapy.

2. The method of claim 1, further comprising recommending, prescribing, or administering anti-immune checkpoint inhibitor therapy if the cancer is determined to benefit from anti-immune checkpoint inhibitor therapy.

3. The method of claim 1, further comprising recommending, prescribing, or administering anti-cancer therapy other than anti-immune checkpoint inhibitor therapy if the cancer is determined to not benefit from anti-immune checkpoint inhibitor therapy.

4. The method of claim 3, wherein the anti-cancer therapy is selected from the group consisting of targeted therapy, chemotherapy, radiation therapy, and/or hormonal therapy.

5. The method of any one of claims 1-4, wherein the control sample is determined from a cancerous or non-cancerous sample from either the patient or a member of the same species to which the patient belongs.

6. The method of any one of claims 1-5, wherein the control sample comprises cells.

7. The method of any one of claims 1-6, further comprising determining responsiveness to anti-immune checkpoint inhibitor therapy measured by at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

8. A method of treating a subject afflicted with a cancer, wherein the cancer comprises at least one activating Janus kinase (JAK) mutation shown in Table 1, comprising administering to the subject anti-immune checkpoint inhibitor therapy, thereby treating the subject afflicted with the cancer.

9. The method of claim 8, wherein the at least one activating JAK mutation comprises an activating JAK3 mutation.

10. The method of claim 9, wherein the activating JAK3 mutation is a JH2 domain mutation, optionally a JAK3^{V722I} or JAK3^{R657Q} mutation, and/or a FERM domain mutation, optionally a JAK3^{S61C} mutation.

11. The method of claim 8, further comprising administering one or more additional anti-cancer agents.

12. The method of claim 11, wherein the one or more additional anti-cancer agent is a JAK or activator thereof.

13. A method of inhibiting hyperproliferative growth of a cancer cell or cells, wherein the cancer cell or cells comprise at least one activating JAK mutation shown in Table 1, comprising contacting the cancer cell or cells with an anti-immune checkpoint inhibitor agent, thereby inhibiting hyperproliferative growth of the cancer cell or cells.

14. The method of claim 13, wherein the step of contacting occurs in vivo, ex vivo, or in vitro.

15. The method of claim 13, wherein the at least one activating JAK mutation comprises an activating JAK3 mutation.

16. The method of claim 15, wherein the activating JAK3 mutation is a JH2 domain mutation, optionally a JAK3^{V722I} or JAK3^{R657Q} mutation, and/or a FERM domain mutation, optionally a JAK3^{S61C} mutation.

17. The method of claim 13, further comprising administering one or more additional anti-cancer agents.

18. The method of claim 17, wherein the one or more additional anti-cancer agent is a JAK or activator thereof.

19. A method of assessing the efficacy of an agent for treating a cancer in a subject, wherein the cancer comprises at least one activating JAK mutation, comprising:
a) detecting in a first subject sample and maintained in the presence of the agent the presence, copy number, amount and/or activity of at least one biomarker listed in Table 1;
b) detecting the presence, copy number, amount and/or activity of the at least one biomarker listed in Table 1 in a second subject sample and maintained in the absence of the test compound; and
c) comparing the presence, copy number, amount and/or activity of the at least one biomarker listed in Table 1 from steps a) and b), wherein the presence or a significantly increased copy number, amount, and/or activity of the at least one biomarker listed in Table 1 in the first subject sample relative to the second subject sample, indicates that the agent treats the cancer in the subject.

20. A method of monitoring the progression of a cancer in a subject, wherein the cancer comprises at least one activating JAK mutation, comprising:
a) detecting in a subject sample at a first point in time the presence, copy number, amount, and/or activity of at least one biomarker listed in Table 1;
b) repeating step a) during at least one subsequent point in time after administration of a therapeutic agent; and
c) comparing the presence, copy number, amount, and/or activity detected in steps a) and b), wherein the presence or a significantly increased copy number, amount, and/or activity of the at least one biomarker listed in Table 1 in the first subject sample relative to at least one subsequent subject sample, indicates that the agent treats the cancer in the subject.

21. The method of claim 20, wherein the subject has undergone treatment, completed treatment, and/or is in remission for the cancer in between the first point in time and the subsequent point in time.

22. The method of claim 20 or 21, wherein the subject has undergone anti-immune checkpoint inhibitor therapy in between the first point in time and the subsequent point in time.

23. The method of any one of claims 20-22, wherein the first and/or at least one subsequent sample is selected from the group consisting of ex vivo and in vivo samples.

24. The method of any one of claims 20-23, wherein the first and/or at least one subsequent sample is obtained from an animal model of the cancer.

25. The method of any one of claims 20-24, wherein the first and/or at least one subsequent sample is a portion of a single sample or pooled samples obtained from the subject.

26. A cell-based method for identifying an agent that inhibits a cancer, the method comprising:
a) contacting a cell expressing at least one biomarker listed in Table 1 with a test agent; and
b) determining the effect of the test agent on the copy number, level of expression, and/or level of activity of the at least one biomarker in Table 1 to thereby identify an agent that inhibits the cancer.

27. The method of claim 26, further comprising determining the effect of the test agent on the copy number, level of expression, and/or level of activity of at least one immune checkpoint inhibitor.

28. The method of claim 26 or 27, wherein said cells are isolated from a source selected from the group consisting of an animal model of a cancer, a subject afflicted with a cancer, and a cell comprising at least one activating JAK3 mutation.

29. The method of any one of claims 26-28, wherein said cells are unresponsive to anti-immune checkpoint inhibitor therapy.

30. The method of any one of claims 26-29, wherein the step of contacting occurs in vive, ex vivo, or in vitro.

31. The method of any one of claims 26-30, further comprising determining the ability of the test agent to bind to the at least one biomarker listed in Table 1 before or after determining the effect of the test agent on the copy number, level of expression, or level of activity of the at least one biomarker listed in Table 1.

32. The method of any one of claims 1-7 and 19-31, wherein the sample comprises cells, cell lines, histological slides, paraffin embedded tissue, fresh frozen tissue, fresh tissue, biopsies, bronchoalveolar lavage (BAL) fluid, blood, plasma, serum, buccal scrape, saliva, cerebrospinal fluid, urine, stool, mucus, or bone marrow, obtained from the subject.

33. The method of any one of claims 1-7 and 19-32, wherein the presence or copy number is assessed by whole exome sequencing, microarray, quantitative PCR (qPCR), high-throughput sequencing, comparative genomic hybridization (CGH), or fluorescent in situ hybridization (FISH).

34. The method of any one of claims 1-7 and 19-32, wherein the amount of the at least one biomarker listed in Table 1 is assessed by detecting the presence in the samples of a polynucleotide molecule encoding the biomarker or a portion of said polynucleotide molecule.

35. The method of claim 34, wherein the polynucleotide molecule is a mRNA, eDNA, or functional variants or fragments thereof.

36. The method of claim 34, wherein the step of detecting further comprises amplifying the polynucleotide molecule.

37. The method of any one of claims 1-7 and 19-32, wherein the amount of the at least one biomarker is assessed by annealing a nucleic acid probe with the sample of the polynucleotide encoding the one or more biomarkers or a portion of said polynucleotide molecule under stringent hybridization conditions.

38. The method of any one of claims 1-7 and 19-32, wherein the amount of the at least one biomarker is assessed by detecting the presence a polypeptide of the at least one biomarker.

39. The method of claim 38, wherein the presence of said polypeptide is detected using a reagent which specifically binds with said polypeptide.

40. The method of claim 39, wherein the reagent is selected from the group consisting of an antibody, an antibody derivative, and an antibody fragment.

41. The method of any one of claims 1-7 and 19-32, wherein the activity of the at least one biomarker is assessed by determining the magnitude of cellular proliferation, cell death, or cytokine production.

42. The method of any one of claims 1-41, wherein the agent or anti-immune checkpoint inhibitor therapy is selected from the group consisting of a blocking antibody, small molecule, antisense nucleic acid, interfering RNA, shRNA, siRNA, aptamer, ribozyme, dominant-negative protein, and combinations thereof.

43. The method of claim 42, wherein the agent is selected from the group consisting of a cytokine, an inhibitor of a Jak kinase inhibitor, a Jak kinase harboring an activating mutation, anti-immune checkpoint inhibitor therapy, and combinations thereof.

44. The method of claim 43, wherein the inhibitor of the Jak kinase inhibitor is an inhibitor of PIAS1, PIAS2, PIAS3, PIAS4, SOCS1, SOCS3, SHP-1, or SHP-2.

45. The method of claim 42, wherein the agent or anti-immune checkpoint inhibitor therapy is selected from the group consisting of inhibitors of PD-1, PD-L1, PD-L2, CTLA-4, and combinations thereof.

46. The method of claim 45, wherein the agent or anti-immune checkpoint inhibitor therapy is a blocking antibody of PD-1, PD-L1, PD-L2, or CTLA-4, and combinations thereof.

47. The method of any one of claims 1-46, wherein the at least one biomarker is selected from the group consisting of 1, 2, 3, 4, 5, 6, 7, 8, 9, 10, or more biomarkers.

48. The method of any one of claims 1-47, wherein the at least one biomarker is an activating JAK3 mutation.

49. The method of claim 48, wherein the activating JAK3 mutation is a JH2 domain mutation, optionally a JAK3^{V722I} or JAK3^{R657Q} mutation, and/or a FERM domain mutation, optionally a JAK3^{S61C} mutation.

50. The method of any one of claims 1-49, wherein the cancer is a solid malignancy.

51. The method of claim 50, wherein the solid malignancy is selected from the group consisting of lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer.

52. The method of any one of claims 1-51, wherein the subject is a mammal.

53. The method of claim 52, wherein the mammal is an animal model of cancer.

54. The method of claim 52, wherein the mammal is a human.
